(19) United States
(12) Patent Application Publication
(10) Pub. No.: US 2014/0030771 A1
(43) Pub. Date: Jan. 30, 2014

(54) COMPOSITIONS AND METHODS FOR INCREASING OIL PRODUCTION AND SECRETION

(71) Applicants: Richard C. Yu, Berkeley, CA (US); Ian Burbulis, Oakland, CA (US); Wayne Riekhof, Lincoln, NE (US); Carlos Gustavo Pesce, San Francisco, CA (US); Leandro Vetcher, Cambridge, MA (US)

(72) Inventors: Richard C. Yu, Berkeley, CA (US); Ian Burbulis, Oakland, CA (US); Wayne Riekhof, Lincoln, NE (US); Carlos Gustavo Pesce, San Francisco, CA (US); Leandro Vetcher, Cambridge, MA (US)

(21) Appl. No.: 13/702,561

(22) PCT Filed: Jun. 8, 2011
(86) PCT No.: PCT/US11/39672

§ 371 Date: Oct. 2, 2013

Related U.S. Application Data
(60) Provisional application No. 61/353,129, filed on Jun. 9, 2010.
(60) Provisional application No. 61/416,235, filed on Nov. 22, 2010.

(51) Int. Cl. C12P 7/64

(52) U.S. Cl. CPC C12P 7/6472
USPC 435/134

(57) ABSTRACT

The present invention provides compositions and methods related to the production of fatty acids, such as triglycerides with genetically engineered cells, such as algae.

### CROSS-REFERENCE

This application claims the benefit of U.S. Provisional Application Nos. 61/353,129, filed Jun. 9, 2010; and 61/416,235, filed Nov. 22, 2010, each of which is incorporated by reference in its entirety.

### BACKGROUND OF THE INVENTION

There is an urgent demand for sustainable and affordable alternatives to petroleum-based fuels. The US biodiesel market reached 600 million gallons and is estimated to grow at a 55% annually. At the same time, biodiesel production is at 20% of the total capacity because of feedstock availability constraints. In addition, the Energy Independence and Security Act and the Renewable Fuels Standard (RFS) set by the federal government commend the increase use of renewable fuels. The RFS mandates blending 46 billion gallons of renewable fuels by 2022. Satisfying this demand will require significant and scalable technologies. Biofuels from algae represent a significant opportunity to impact the U.S. energy supply for transportation fuels. Despite their potential, the state of technology for producing algal fuels is in its infancy and there is no established affordable and scalable production process at commercial scale.

For biofuels to displace even a moderate amount of fossil fuels used in the transportation sector requires development of an abundant source of triglycerides (TAGs). TAGs from current oilseed crops and waste oils would barely dent annual U.S. diesel demand. The U.S. Diesel demand is approximately 60 billion gallons per year (bgy), but estimations from the National Bioenergy Center indicate that the entire U.S. soybean crop could only provide approximately 2.5 bgy and that the world-wide production of biodiesel from all oilseed crops would yield only 13 bgy. Algae appear to be the only worldwide feedstock capable of replacing crude oil in cost and scale.

Microalgae are microscopic aquatic plants that carry out the same process and mechanism of photosynthesis as higher plants. Microalgae convert sunlight, water and carbon dioxide into biomass and oxygen. Algae have long been recognized as an alternative source of oil for the production of biofuels. Algae have a much higher productivity potential than terrestrial biofuels. Yields are approximately ten times that of terrestrial crops (depending on crop). Experts suggest that 2,000 gallons per acre per year would be a significant accomplishment and estimate that with future technologies a productivity of 6,000 gallons per acre can be achieved.

However, the promise of algae biofuels has not yet been realized. Current methods to produce biofuels from algae are extremely limited by low oil yields in fast growing strains (and slow growth of strains with high oil yields), difficulties in algae cell harvesting and oil extraction, and contamination when algae is farmed in large-volume open-ponds. In addition, standard processes used to stress/starve the cells to activate responses that increase the levels of oil by weight per cell (typically nitrogen starvation) also dramatically reduce the rate of cell division, causing little or no net gain in total synthesized oil in a given volume of cell culture. Most importantly, many key strategies to modify or engineer algae to overcome these limitations have remained out of reach because algae are plagued by low and variable expression of transgenes incorporated into the nuclear genome.

The present invention provides compositions and methods related to the production of fatty acids, such as triglycerides, with genetically engineered cells, such as algae.

DHA and EPA are two common long-chain ω-3 Fatty Acids (FA). Many studies confirm the copious benefits of an adequate supply of ω-3 FAs, since they support brain, eye and heart health throughout all stages of life. The strongest and most established body of science for ω-3 FAs is in relation to cardiovascular health and cognitive performance. The National Institute of Health has recommended daily targets for minimal DHA intakes for children, pregnant woman, and adults. DHA and EPA can be obtained from animal sources, like fish oil, and from vegetarian sources, such as algae. However, vegetarian sources of DHA are significantly better because they are sustainable, do not add unpleasant fish odor, and are free of toxic impurities such as PCBs and mercury.

However, ω-3 FAs are nutritionally essential but not affordable to large segments of the population. There is an urgent need for a platform for low-cost and sustainable DHA and EPA production that will make the health benefits of ω-3 fatty acids (FAs) available to a significantly broader section of the population.

Pharmaceutical-grade compositions of ω-3 FAs can be prescribed to help lower cholesterol. Pharmaceutical grade EPA/DHA oil has a high potency because of its high EPA and DHA content, it has very little oxidation and has had impurities such as PCB's and mercury removed. The refining process necessary to produce pharmaceutical grade EPA/DHA oil from fish oil is very extensive and costly. Several steps required and repeated several times in the production of pharmaceutical grade include, the removal of free FAs and impurities, the removal of environmental pollutants and cholesterol, the formation of ethyl esters, and evaporation and condensation to increase ω-3 FAs concentration.

The present invention provides compositions and methods related to the production of ω-3 FAs, such as DHA and EPA, with genetically engineered cells, such as algae.

Retinol, the animal form of vitamin A, is a fat-soluble vitamin important in vision and bone growth. Retinol is among the most useable forms of vitamin A, which also include retinal (aldehyde form), retinoic acid (acid form) and retinal ester (ester form). These chemical compounds are collectively known as retinoids. Microalgae can synthesizes a relatively large amount of β-carotene and other carotenoid derivatives, such as lutein, loroxanthin, and the xanthophylls neoxanthin and violaxanthin. All these accumulate to about 1 mg/l of standard medium density culture.

A rapidly growing use of retinoids is as cosmeceuticals. Cosmeceuticals are a marriage between cosmetics and pharmaceuticals. Like cosmetics, cosmeceuticals are topically applied, but they contain ingredients that influence the biological function of the skin. In particular, there is an increased interest of natural and sustainable sources of chemical ingredients for the cosmetic industry.

The present invention provides compositions and methods related to the production of retinoids, such as retinol, from carotenoids with genetically engineered cells, such as algae.

### SUMMARY OF THE INVENTION

In one aspect, the present invention provides a metabolic engineered cell, wherein the cell is engineered by: over-expressing a di-acylglycerol acyltransferase 2 (DGAT2) gene in the cell; or inhibiting a gene in the starch synthesis pathway in the cell. In some embodiments, the cell is engineered by the introduction of a gene into the nuclear genome. In some embodiments, the cell is engineered by down-regulation of a gene, for example by introduction of DNA that causes RNAi silencing of the target gene. In some embodiments, the expression of the introduced gene is stable, such for at least 9 months on solid media. In some embodiments, the DGAT2 gene is selected from the group consisting of: a human DGAT2 gene, and a Chlamydomonas DGAT2 gene, and a homologue thereof. In some embodiments, the DGAT2 gene has an amino acid sequence that is at least 90% identical to an amino acid sequence selected from the group consisting of SEQ ID NOs: 11-50. In some embodiments, the DGAT2 gene has an amino acid sequence selected from the group consisting of SEQ ID NOs: 11-50. In some embodiments, the cell is a Chlamydomonas cell, such as UVM4 or UVM11. In some embodiments, the gene in the starch synthesis pathway is STAG.

In another aspect, the present invention provides a method for producing a lipid in a cell, comprising: culturing a metabolic engineered cell to produce a lipid. In some embodiments, the lipid comprises triacylglycerol (also called triglyceride). In some embodiments, the metabolic engineering is selected from the group consisting of: over-expressing a di-acylglycerol acyltransferase 2 (DGAT2) gene in the cell; and inhibiting a gene in the starch synthesis pathway in the cell. In some embodiments, the cell is grown under a condition where the cell growth rate is affected less than 25% in comparison of a cell that is not metabolic engineered.

In yet another aspect, the present invention provides a method for producing a lipid, comprising: culturing a genetically engineered cell; and producing a lipid secreted from the genetically engineered cell. In some embodiments, the lipid comprises triglycerides. In some embodiments, the lipid is in the form of a lipid droplet. In some embodiments, the lipid is secreted in the form of a fat globule. In some embodiments, the lipid is secreted in the form of a vesicle. In some embodiments, the cell is transformed with and stably expresses one or more gene selected from the group consisting of: BTN, Syntaxin, FMG1-B, Cbl1, Fox1, IFT20, ADPH, Xor, MLDP, and AAM-B, and a fragment thereof. In some embodiments, the cell is an alga cell or a yeast cell. In some embodiments, the cell is a Chlamydomonas, such as UVM4 or UVM11.

In a further aspect, the present invention provides a genetically engineered cell, wherein the cell secrets a lipid through a non-toxic mechanism. In some embodiments, the lipid is secreted in the form of a lipid droplet. In some embodiments, the lipid is secreted in the form of a vesicle. In some embodiments, the lipid is secreted in the form of a fat globule. In some embodiments, the secreted lipid is enclosed by flagellar membranes. In some embodiments, the lipid comprises a triglyceride. In some embodiments, the cell is an alga stably transformed with one or more genes encoding a protein selected from the group consisting of: BTN, Syntaxin, FMG1-B, Cbl1, Fox1, IFT20, Xor, ADPH, MLDP, and AAM-B, and a fragment thereof.

In one aspect, the present invention provides a composition, comprising a droplet comprising triglyceride. In some embodiments, the composition further comprises one or more proteins selected from the group consisting of: BTN, Syntaxin, FMG1-B, Cbl1, Fox1 IFT20, Xor, ADPH, MLDP, and AAM-B, and a fragment thereof.

In one aspect, the present invention provides a composition, comprising: triglyceride, and an alga transformed with a gene that is stably integrated and expressed in the alga, or debris of the alga. In some embodiments, the gene is DGAT2. In some embodiments, the gene is integrated in the nucleus of the alga.

In another aspect, the present invention provides a cell comprising an exogenous promoter having the nucleotide sequence selected from the group consisting of SEQ ID NOs:1-10.

In another aspect, the present invention provides a vector comprising a promoter having the nucleotide sequence selected from the group consisting of SEQ ID NOs:1-10.

In another aspect, the present invention provides a method for producing retinol or for increasing the production of retinol, comprising: culturing a genetically engineered cell to produce retinol, wherein the cell is transformed with either one or both of these two genes: a β-carotene: oxygen 15,15′-monooxygenase gene and a aldehyde NAD(P)H reductase gene.

In another aspect, the present invention provides a method for producing DHA, comprising: culturing a genetically engineered cell to produce DHA. In some embodiments, the cell is transformed with a fatty acid elongase gene and a fatty acid desaturase gene. In some embodiments, the cell is transformed with a Δ6-desaturase gene, a Δ6-elongase gene, a Δ5-desaturase gene, a Δ5-elongase gene, and a Δ4-desaturase gene. In some embodiments, the production of DHA is by the expression of one or more genes selected from the group consisting of: Fat-3, Elo-2, Fat-4, elo, and IgD4. In some embodiments, the method comprises converting naturally occurring C18:3 (18:3Δ9, 12, 15) to C20:4 (C20:4Δ8,11,14,17). In some embodiments, the cell is a Chlamydomonas, such as UVM4 or UVM11.

In another aspect, the present invention provides an alga transformed with either one or both of these two genes: a β-carotene: oxygen 15,15′-monooxygenase gene and a aldehyde NAD(P)H reductase gene.

In another aspect, the present invention provides an alga transformed with a fatty acid elongase gene and a fatty acid desaturase gene. In some embodiments, the alga is transformed with a Δ6-desaturase gene, a Δ6-elongase gene, a Δ5-desaturase gene, a Δ5-elongase gene, and a Δ4-desaturase gene. In some embodiments, the alga is a Chlamydomonas cell, such as UVM4 or UVM11.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the metabolic pathways related to the synthesis and degradation of triacylglycerol and some of the metabolic engineering approaches to increase oils described in the claims of this patent. Carbon fixed through photosynthesis is converted to either of two major storage forms, starch and triacylglycerol (i.e., oil). We have engineered increased carbon flow into oils by reducing the activity of Sta6 (for example, by down-regulating gene expression via RNAi), which is absolutely required for starch synthesis. We have also engineered increased carbon flow into oils by increasing DGAT2 activity (for example, by overexpression of native DGAT2 genes or DGAT2 orthologs).

FIG. 2 A-D: The UVM strains enable reliable eukaryotic algae nuclear genetic engineering. 2A: (from Neupert et al, 2008). Analysis of GFP expression in a number of representative transformants by Western blotting using an anti-GFP antibody. Elow47 is a wild-type alga and UVW4 and UVM11 are mutant strain that express transgenes at high levels. All strains are transformed with expression plasmid containing GFP gene and a selectable marker and were confirmed to have intact GFP expression cassettes by PCR. Isolates of UVM4 (#4, #14, and #15) and UVM11 (#8, #9, and #17) transformed with GFP expression vector (pJR38 from Neupert et al., 2008) express significant amounts of GFP. 2B: Flow cytometry of untransformed UVM4 cells (top left), UVM4 transformed with a GFP expression plasmid and expressing GFP (top right), and a mixed population of non-expressing and expressing cells (bottom). Each panel shows GFP fluorescence (Y axis) plotted against PerCP fluorescence (a measure of chlorophyll and carotenoid autofluorescence). 2C: Transgene expression is stable for at least 6 months. We compared GFP expression in Elo47, UVM4, and UVM11 freshly transformed with pJR38 (from left, bars 2 and 3) with UVM4 and UVM11 cells transformed at least 6 months ago with pJR38 (bars 4 and 5). GFP expression levels are comparable. 2D: Frequency of UVM4 and UVM11 transformants that express high transgene levels is much higher. We selected 35 pJR38 transformants each of Elo47, UVM4, and UVM11, measured GFP expression by flow cytometry, and tallied the number of transformants whose popuations expressed significant amounts of GFP (>25% of cells were in the region of the GFP vs PerCP plot corresponding to GFP expression (see top right panel in FIG. 2B).

FIG. 3: DGAT2 expression increases oil without affecting the cell growth rate. A: Anti-HA tag Western blot. ctrl: untransformed UVM4 control 14d and 28d: Isolate #13 of transformation experiment of UVM4 with a plasmid expressing C-terminal HA-tagged DGAT2 gene from Chlamydomonas reindhartii at high levels, grown in culture for 14 and 28 days respectively B: Total Nile Red fluorescence measured in a flow cytometer of the same two strains. Nile Red localizes to lipid droplets and fluoresces only when embedded in oil. C: Quantification of the experiment shown in 3B. D: Growth curves of the same two strains. Values are averages of 3 independent cultures. Errors bar are standard error of the mean.

FIG. 4: STA6 knock-down by RNAi increases oil and reduces starch without affecting the cell growth rate. A: High frequency of effective RNAi among transformants in the UVM4 strains. The unmutagenized parental Elo47 and the transgene expressing strain UVM4 were transformed with plasmid pGPB1062, which drives the expression of a double stranded RNA with an ˜800 bp fragment of the STA6 cDNA sequences. Starch was measured in transformants using a commercial starch assay kit (SIGMA). White column: Transformants with normal starch amounts. Black column: Transformants with lower starch content. All 23 Elo47 transformant were found to have normal starch levels, while 3 of 18 UVM4 transformants had lower starch content. B: Starch measurements using a commercial kit (SIGMA). WT: untransformed UVM4. sta6⁻: mutant strain CC-4333, obtained from the Chlamydomonas Center (www.chlamy.org), carrying a loss of function mutation in STA6. STA6 RNAi: One of the three UVM4 pGPB1062 transformants with low starch. C: TAG measurements on the same strains as in B, using a using a commercial kit that correctly compensates for background glycerol levels (Sigma). D: Growth curves of the strains shown in B and C. Values are averages of 3 independent cultures. Errors bar are standard deviations.

FIG. 5A depicts a proposed mechanism of lipid droplet (LD) secretion in the mammary milkfat secretion system. 1. Secretion proteins target and bind LD to cell membrane: butyrophilin (BTN), an LD membrane protein, binds to adipophilin (ADPH), a plasma membrane (PM) protein., 2. Binding between secretion proteins drive envelopment of LD by cell membrane: a large number of BTN-ADPH pairs drives the zipping-up of the two membranes, 3. Membrane stresses and high curvature at the neck of the budding LD favors membrane fusion and pinching off of the PM-enveloped LD, and 4. LD (oil) secreted. It has been observed that BTN also localizes to the plasma membrane and that two molecules of BTN can interact and bind each other forming BTN-BTN pairs that could potentially link the LD and the PM together. This observation has led to an alternative proposal for the secretion in which both the PM and the LD membrane protein are BTN molecules. 5B: Engineering accumulation of lipids in the flagella: Lipids are transported into the flagella via fusion proteins containing domains from a flagellar transport protein such as IFT20 and domains or localization peptides from a lipid droplet potein such as MLDP (left panel), or targeted to the flagellar membrane by similar fusion proteins containing domains of flagellar membrane proteins such as Fmg1B instead of domains from a flagellar transport protein (right panel). 5C: Lipid droplet secretion by deflagellation. Flagella, loaded with lipid droplets, either by an IFT20-like mechanism (shown) or by an Fmg1B-like mechanism (not shown), detach from the cell via deflagellation (left flagella). During or after deflagellation, there may be some release of lipid droplets into the external media (right flagella). 5D: Lipid droplet secretion during flagellar resorption. After disassembly of the axoneme (left panel), part or all of the flagellar membrane is separated by the cell (right panel), and may contain trapped lipids. There may also be release of lipid droplets into the external media.

FIG. 6A-6B depict the reconstruction of lipid droplet secretion system in heterologous systems such as algae, via the expression of foreign proteins from transgenes. The proteins in these engineered systems are BTN, ADPH or other proteins that would carry out the LD-PM zipping and pinching off processes. Plasma membrane targeted Proteins: BTN, Syntaxin, calcineurin B like protein 1 (cbl1), multicopper ferroxidase (FOX1), FMG1-BLipid Droplet targeted proteins: ADPH, MLDP, AAM-B, BTN. 6A: Two-component system approach: an LD membrane protein interacts with a PM protein, the formation of numerous pairs drives budding and secretion as in the mammary secretion model in FIG. 5. 6B: One-component system approach. A single polypeptide carries two membrane localization signals, one that targets the protein to the LD membrane and a second one that targets the protein to the PM. In this system multiple molecules of the same polypeptide cause both membranes to come apposed, to bud and eventually to then to pinch off.

FIG. 7A-B depicts examples of localization of protein components of the lipid droplet secretion system. Shown are spinning disk fluorescence microscope images. A: Localization to the plasma membrane. Panels show fluorescence measured using GFP filters minus the normalized autoflorescence signal measured with Nile Red filter settings (mainly chlorophyll fluorescence). Images are of UVM4 cells transformed with: I. Empty vector control. II: Cytosolic GFP expression vector, pJR38 (from Neupert et al., 2008). III vector expressing GFP fused to the membrane-localization domain from all. Increased fluorescence is visible at plasma membrane relative to control. B: Localization to lipid droplets. Panel show images with the indicated filter sets. Bottom images show background autofluorescence. Top images show background plus specific signal. White arrows point to lipid droplets. UVM4 cells were I: Stained with Nile Red. II: Transformed with empty vector control. III. Transformed with vector expressing GFP fused to MLDP. White arrows point to circles of green fluorescence consistent with lipid droplets labeled on the surface with GFP.

FIG. 8 depicts an exemplary engineered DHA synthesis pathway, with exemplary enzymes used in each step. FA: fatty acids, DHA: Docosahexanoic acid (all-cis-docosa-4,7,10,13,16,19-hexa-enoic acid), ALA: alpha-linolenic acid (all-cis-9,12,15-octadecatrienoic acid).

FIG. 9 depicts the biosynthesis of retinol from β-carotene.

FIG. 10 A-C: Exemplary promoters according to the embodiments of the present invention. 10A: List of genes from which the exemplary promoters are drawn. 10B-C: Sequence of the exemplary promoters from the genes listed in 10A.

FIGS. 11A-11F depict the exemplary amino acid sequences of the DGAT2 genes according to the embodiments of the present invention.

FIG. 12 depicts the phylogram of DGAT2 sequences.

FIG. 13 depicts the exemplary amino acid sequences of all known C. reinhardtii DGAT2 orthologs according to the embodiments of the present invention.

FIGS. 14A-14C depict the exemplary amino acid sequences of the BTN1A1, Syntaxin, IFT20, Clb1, Fox1, MLDP, ADPH, XOR, Fmg-1B, and AAM-B genes according to the embodiments of the present invention

FIGS. 15 A-B. A: AAMBpep targets GFP to lipid droplets in Chlamydomonas. Spinning disk confocal fluorescence images of GFP and Nile Red channels are shown. Chloroplast autofluorescence appears in both channels. In cells expressing AAMBpep-GFP (right) circles of green fluorescence are also visible (white arrows), as expected for a fluorescent protein that localizes to the lipid droplet membrane. B: Flagellar secretion transgenes cause accumulation of extracellular TAG. The ratio of extracellar TAG to total TAG is plotted. 2 strains (#2 and #7) expressing the IFT20-AAMB-Hemaglutinin-tag (IAH) secretion construct were >2-fold higher than control strains: untransformed UVM4 cells, UVM4 transformed with a empty control vector, and a bld2 mutant strain that has no flagella.

FIG. 16: Flow diagrams of production systems for algae that secrete oil. A: High rate ponds with inducible secretion. Process starts on the Inoculation Ponds (top-left). Flow is represented by lines and arrows: black dashed lines for aqueous media, black solid lines for algal cells (i.e. biomass), grey solid lines for oil produced and gray dashed line for CO2 and waste. Oil produced is obtained at the end of the process, bottom-middle. Note that the biomass cycles continuously in the center part of the diagram. Individual stations are described in the main text. B: Photobiorreactor with constant secretion. Process starts in the Inoculation Reactor (top-left). Lines and arrows as in panel A. Biomass also cycles continuously.

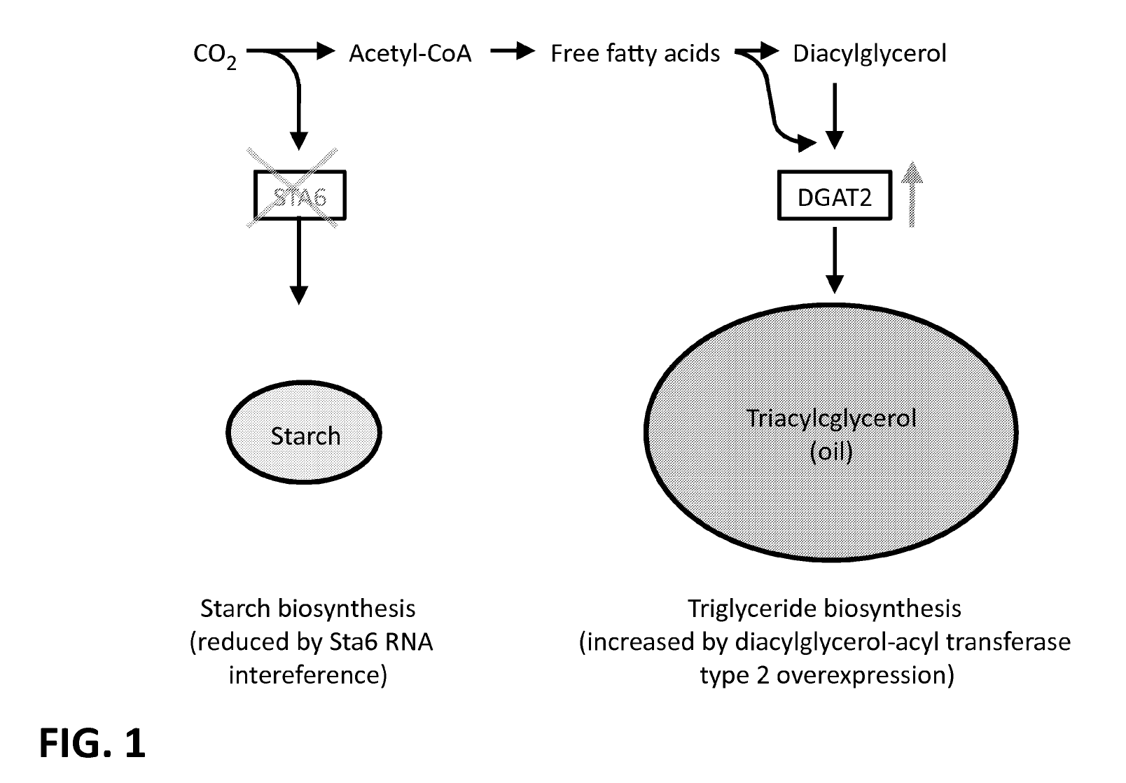

### DETAILED DESCRIPTION OF THE INVENTION

The present invention is directed to compositions and methods for the production of oil triglycerides by excretion using genetically engineering organisms (e.g. photosynthetic algae) through a non-toxic mechanism. The present invention enables the increase of oil synthesis in genetically engineered photosynthetic algae without the tradeoffs in growth rates. The present invention also enables the secretion of the oil to avoid cell harvesting and oil extraction, thus enabling more continuous and efficient oil production. The present invention enables the production of retinol and DHA in genetically engineered eukaryotic algae. The present invention enables genetic tools for the modulation of nuclear gene expression in eukaryotic algae

### I. Metabolic Engineered Cells

In one aspect, the present invention provides a metabolic engineered cell.

### A. The Cells

The cells metabolically engineered according to the methods of the present invention are different types and from different organisms, include, but are not limited to, bacteria, fungi (e.g. yeast), algae, plants, and animals.

In some embodiments, the cell is a microorganism, such as yeasts or a microalga.

By “algae” herein is meant any organisms with chlorophyll and a thallus not differentiated into roots, stems and leaves, and encompasses prokaryotic and eukaryotic organisms that are photoautotrophic or photoauxotrophic. The term “algae” includes macroalgae (commonly known as seaweed) and microalgae. For certain embodiments of the invention, algae that are not macroalgae are preferred. The terms “microalgae” and “phytoplankton,” used interchangeably herein, refer to any microscopic algae, photoautotrophic or photoauxotrophic eukaryotic algae.photoautotrophic or photoauxotrophic protozoa, photoautotrophic or photoauxotrophic prokaryotes, and cyanobacteria (commonly referred to as blue-green algae and formerly classified as Cyanophyceae). The use of the term “algal” also relates to microalgae and thus encompasses the meaning of “microalgal.” The term “algal composition” refers to any composition that comprises algae, and is not limited to the body of water or the culture in which the algae are cultivated. An algal composition can be an algal culture, a concentrated algal culture, or a dewatered mass of algae, and can be in a liquid, semi-solid, or solid form. A non-liquid algal composition can be described in terms of moisture level or percentage weight of the solids. An “algal culture” is an algal composition that comprises live algae.

In some embodiments, the algae is Chlamydomonas, such as the unicellular green alga Chlamydomonas reinhardtii. In some embodiments, the alga is the Chlamydomonas reinhardtii strains UVM4 or UVM11 that were identified to be able to express transgenes efficiently. Neupert, J. et al., Plant J. 57:1140-1150 (2009) and WO2009141164A1.

Other organisms suitable for the present invention are yeast (e.g. Saccharomyces cerevisiae).

### B. Metabolic Engineering

By “metabolic engineering” herein is meant the targeted and purposeful alteration of metabolic pathways in an organism in order to better understand and use cellular pathways for chemical transformation, energy transduction, and supramolecular assembly. A metabolic pathway, or biosynthetic pathway, in a biochemical sense, can be regarded as a series of chemical reactions occurring within a cell, catalyzed by enzymes, to achieve either the formation of a metabolic product to be used or stored by the cell, or the initiation of another metabolic pathway (then called a flux generating step). Many of these pathways are elaborate, and involve a step by step modification of the initial substance to shape it into a product having the exact chemical structure desired.

Metabolic engineering can be divided into two basic categories: modification of genes endogenous to the host organism to alter metabolite flux and introduction of foreign genes into an organism. Such introduction can create new metabolic pathways leading to modified cell properties including but not limited to synthesis of known compounds not normally made by the host cell, production of novel compounds (e.g. polymers, antibiotics, etc.) and the ability to utilize new nutrient sources.

In some embodiments, metabolic engineering is accomplished by introducing one or more exogenous genes in a metabolic pathway into a host cell by transgenesis. In some embodiments, the exogenous gene replaces an endogenous gene. In some embodiments, the exogenous gene is introduced to complete a metabolic pathway partially exist in the host cell. In some embodiments, the exogenous gene is introduced to provide a metabolic pathway that is not exist in the host cell without the genetic engineering. In some embodiments, gene expression is down-regulated by expression of DNA that causes RNAi silencing of the target gene.

Methods of modifying gene expression or introducing one or more exogenous genes into a cell are known in the art. For example, methods of stably transforming algal species and compositions comprising isolated nucleic acids of use are well known in the art and any such methods and compositions may be used in the practice of the present invention. Exemplary transformation methods of use may include microprojectile bombardment, electroporation, protoplast fusion, PEG-mediated transformation, DNA-coated silicon carbide whiskers or use of viral mediated transformation (see, e.g., Sanford et al., 1993, Meth. Enzymol. 217:483-509; Dunahay et al., 1997, Meth. Molec. Biol. 62:503-9; U.S. Pat. Nos. 5,270,175; 5,661,017).

### C. Nuclear Transgene Expression

In some embodiments, the metabolic engineered cells are generated by nuclear transgene expression. By “nuclear transgene expression” herein is meant that a gene introduced in the nuclear genome of a host cell is expressed into an RNA which may code for a protein or have a function on its own (e.g as an RNAi knock-down molecule).

Expression of nuclear transgenes in algae is typically inconsistent, unpredictable, very weak, and transient. Enhanced protein expression from nuclear transgenes using strong gene promoters and optimized codon usage typically yield only very low levels of expression in a small percentage of transformants. Gene knock-downs by RNAi induced by expression of complementary RNAs from nuclear transgenes are also very inefficient. Obtaining a strain with a gene knocked down by RNAi requires the screening of hundreds of transformants. Such screenings often fail to yield any isolate displaying reduced expression of the gene targeted by the RNAi transgene. It is likely that the inefficiency of RNAi derived from nuclear transgenes results from the low amounts of RNA expressed from the nuclear transgenes.

For example, nuclear transgene expression in Chlamydomonas reinhardtii, one of the most widely used and best understood eukaryotic algae species, is usually weak, inconsistent between different cells, and unstable. It has been tried to address these challenges in algae transgenics by using chloroplast gene expression, but proteins expressed from the chloroplast genome cannot gain potentially critical post-translational modifications and are spatially restricted to the chloroplast.

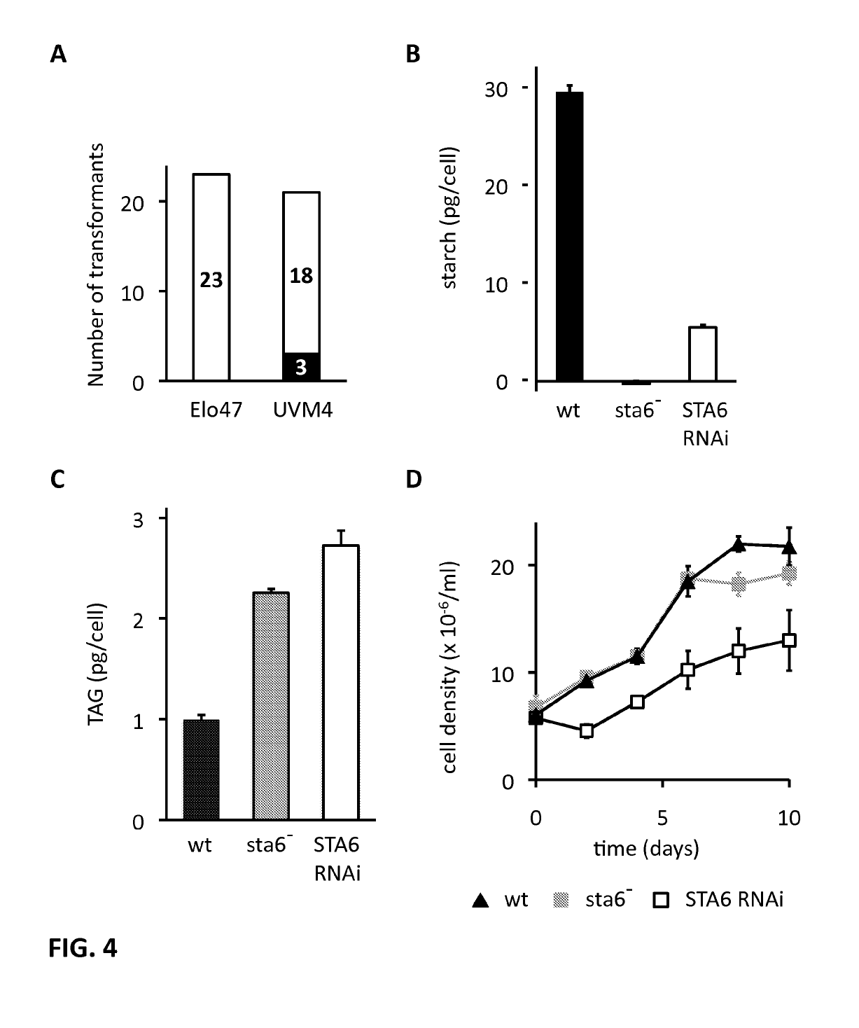

The obstacles to algae genetic engineering posed by the poor expression of nuclear transgenes were recently overcame due to the generation of mutant C. reinhardtii strains (UVM strains) that consistently express transgenes at very high levels when driven by strong promoters. Neupert, J. et al., Plant J. 57:1140-1150 (2009). The UVM strains grow at the same rate as wild type cells. The UVM strains are also more efficient than wild type cells at generating strains with reduced gene expression of genes targeted by RNAi molecules expressed from nuclear transgenes (FIG. 4). Algae that consistently express transgenes at different levels from the nucleus (which, in contrast to expression from the chloroplast, allows for the targeting of proteins to different locations in the cell) and that yield RNAi gene knock-downs with easy present a novel platform for metabolic engineering.

In some embodiments, the metabolic engineered cell of the present invention is a Chlamydomonas reinhardtii that consistently and stably expresses nuclear transgenes at higher levels than other algae strains expressing a transgene from the nucleus. In some embodiments, in the cells of the invention, foreign protein accumulation levels are essentially uniform amongst all transformants, not significantly influenced by the integration location in the nuclear genome and largely independent of codon usage adaptation. In some embodiments, gene expression of selected genes is reduced by high levels of RNAi molecules expressed from nuclear transgenes.

Methods for generating metabolic engineered cell with nuclear transgene expression are carried out according the methods described herein or those known in the art Kindle, KL, Proc Natl Acad Sci USA. 87(3): 1228-1232 (1990) In some embodiments, cells with nuclear transgenes are generated according to the method disclosed in Neupert, J. et al., Plant J. 57:1140-1150 (2009) and WO2009141164A1.

In some embodiments, one or more exogenous genes are introduced into the host cells using a vector. In general, the vector comprises the nucleotide sequences encoding the exogenous gene and the regulatory elements necessary for the transformation and/or expression of gene in the host cell, such as the promoter sequences provided herein.

In some embodiments, the vectors of the present invention comprise a backbone sequence.

In some embodiments, the vectors of the present invention comprise a multiple cloning site, one or more regulatory elements to control the expression of the insert gene, as well as one or more markers for selection. Markers included are paromomycin resistance (Sizova et al., Gene 181:13-8 (1996)) and hygromycin B resistance (Berthold et al., Protist 153:401-12 (2002).

In some embodiments, the vectors of the present invention comprise a signal peptide that direct the localization of the protein to a desired location within the cell. The transit peptides include ZEP1 from C. reinhardtii (XM_{—}001701649.1), CHYB from C. reinhardtii (XM_{—}001698646.1), PETF from C. reinhardtii (XM_{—}001692756.1), HLP from C. reinhardtii (NW_{—}001843472.1)

The nucleotide sequences encoding the proteins to be introduced into the host are either the sequence known in the public domain or are designed to encode such proteins. In some embodiments, the nucleotide sequences are codon optimized according to the host cells. A summary of codon usage of C. reinhardtii is provided in Mayfield and Kindle, PNAS (1990) 87:2987-2091.

### D. Promoters

Because Chlamydomonas nuclear gene expression is usually so problematic without using the UVM strains developed by Bock and coworkers, most of the expression vectors that have been developed use “strong” promoters. Rational metabolic engineering depends on tuning the expression levels of transgenes. For example, it is possible that too much expression of DGAT2 (see below) might affect the health of the cells and diminish growth rates such that, per unit culture volume, the net rate of TG synthesis (amount per cell X number of cells per unit time) is less than optimal.

In one aspect, the present invention provides a panel of promoters that consistently and predictably express genes at three different levels over at least three orders of magnitude (strong, medium, weak).

Exemplary strong promoters are the promoters from the following genes: Photosystem II stability/assembly factor, Peptidyl-Prolyl cis-trans isomerase, histidinol dehydrogenase, malate dehydrogenase (NAD+) (Mdh2), and LHC (LhcII-1.3).

Exemplary medium promoters are the promoters from the following genes: Formate Nitrite transporter, ATP-dependent CLP protease proteolytic subunit, serine carboxypeptidase I, and 40S ribosomal protein S19.

Exemplary weak promoter is the promoter from the following gene: sterol-C-methyltransferase Erg6 like protein.

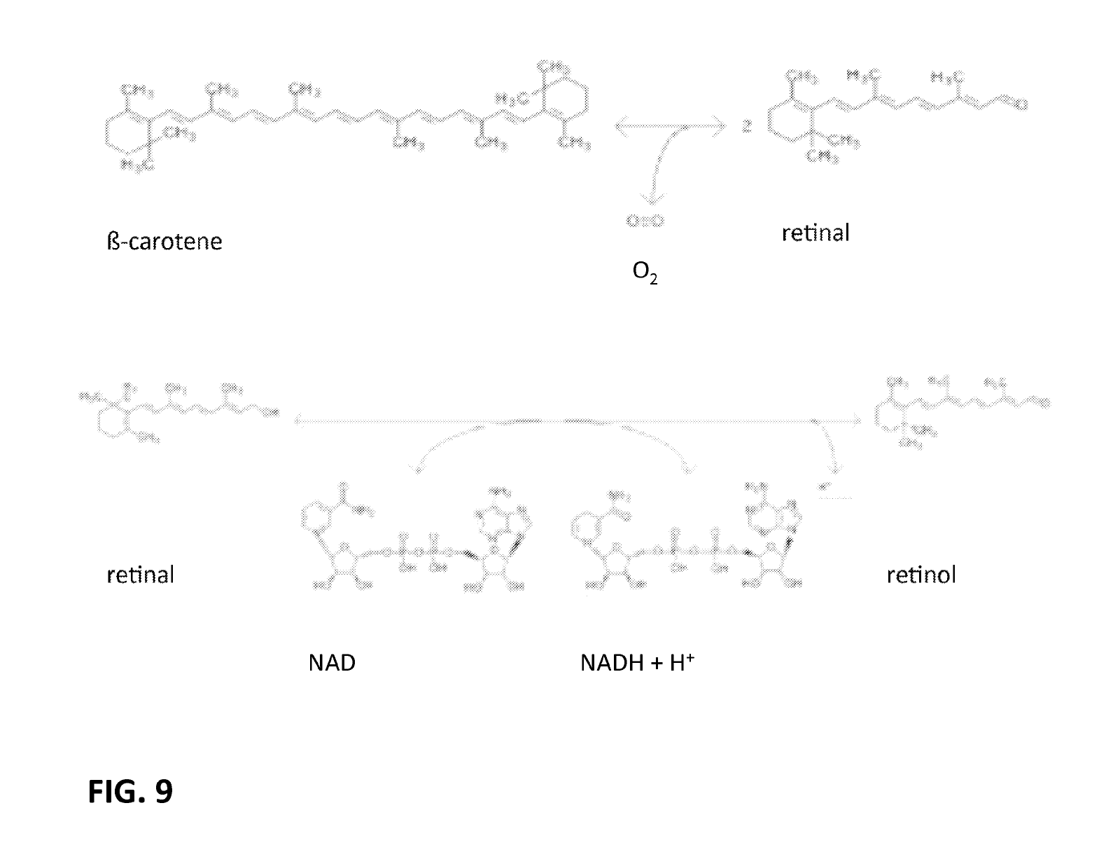

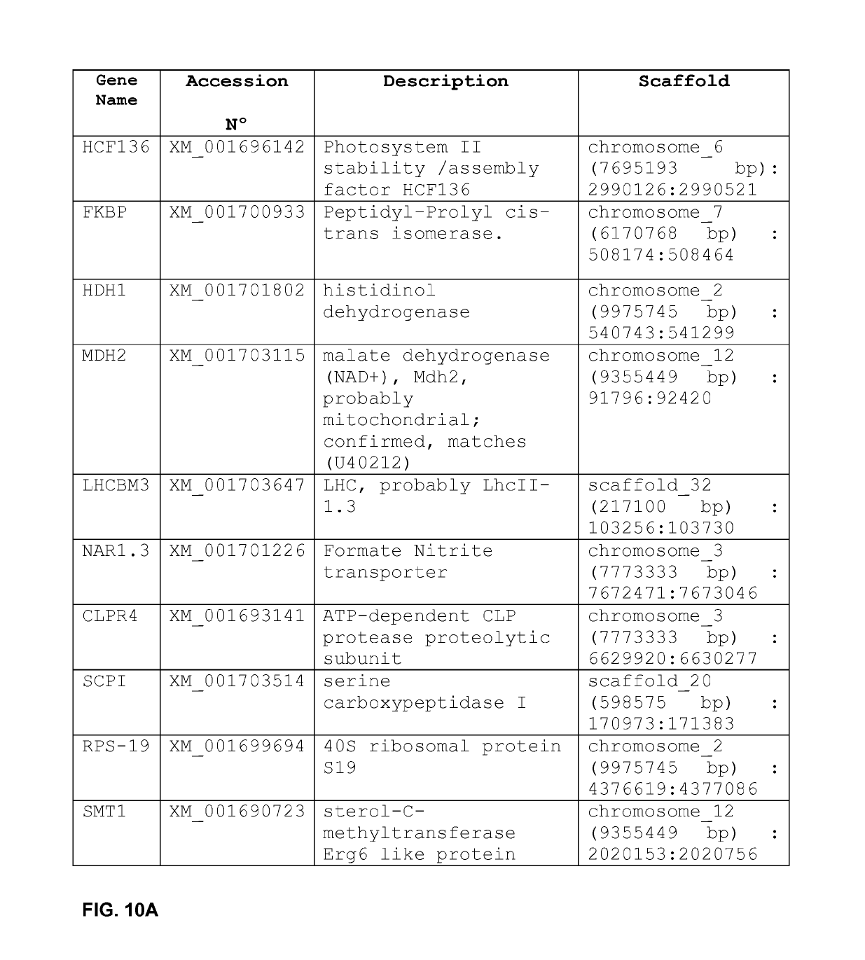

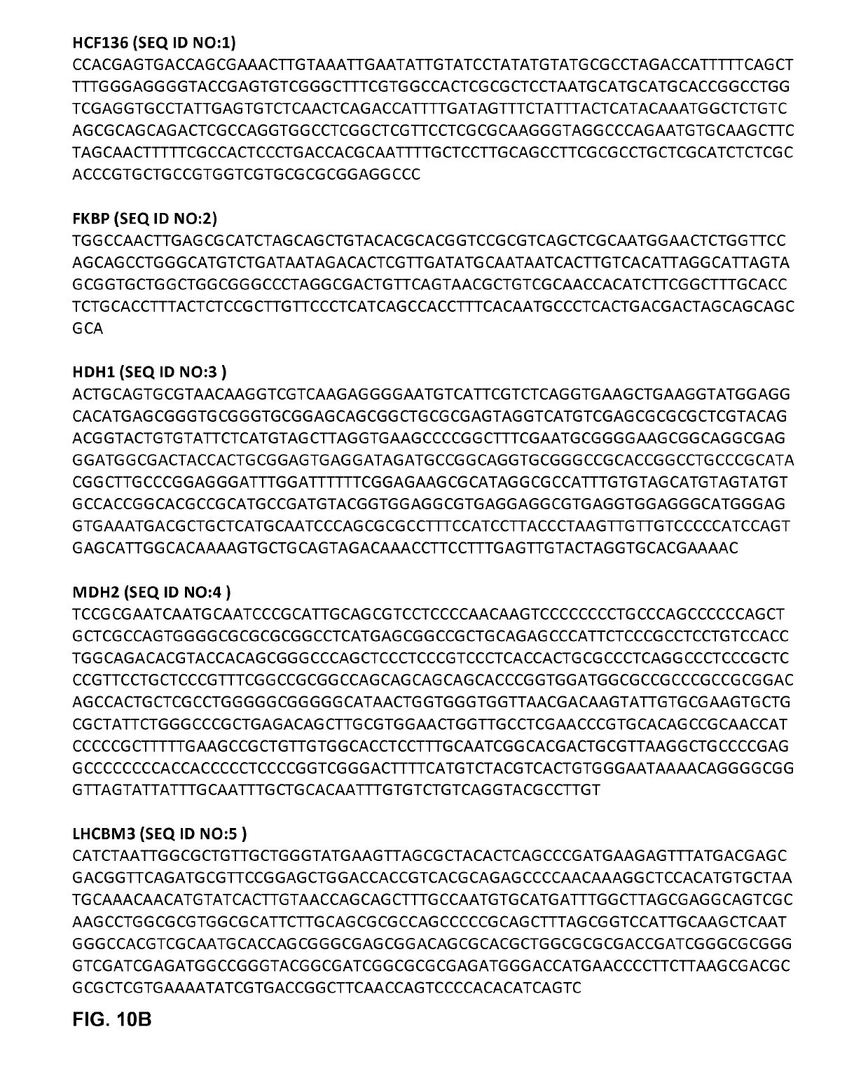

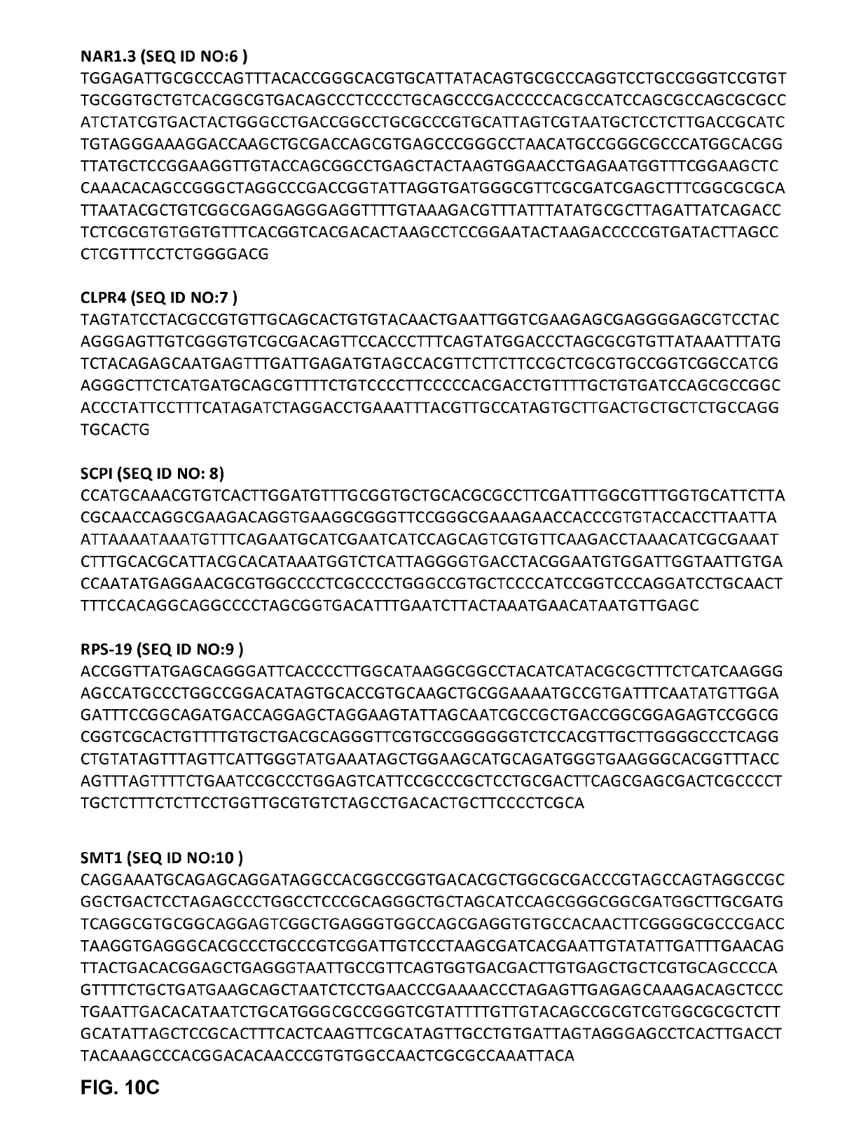

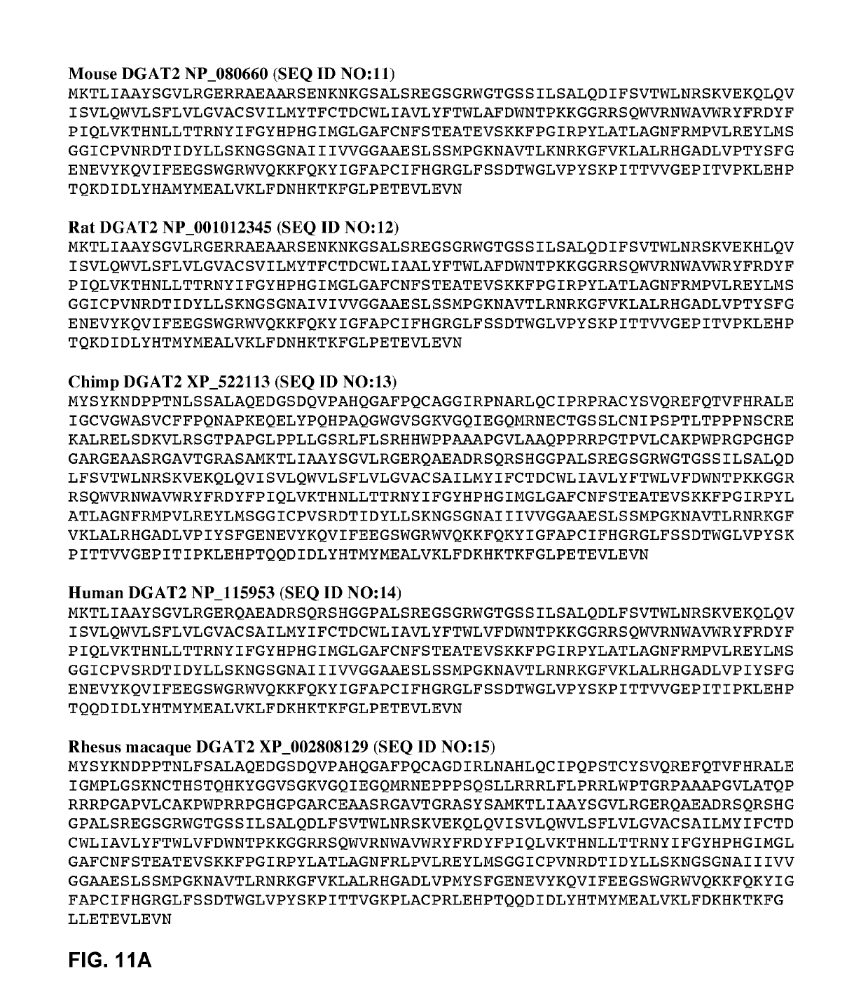

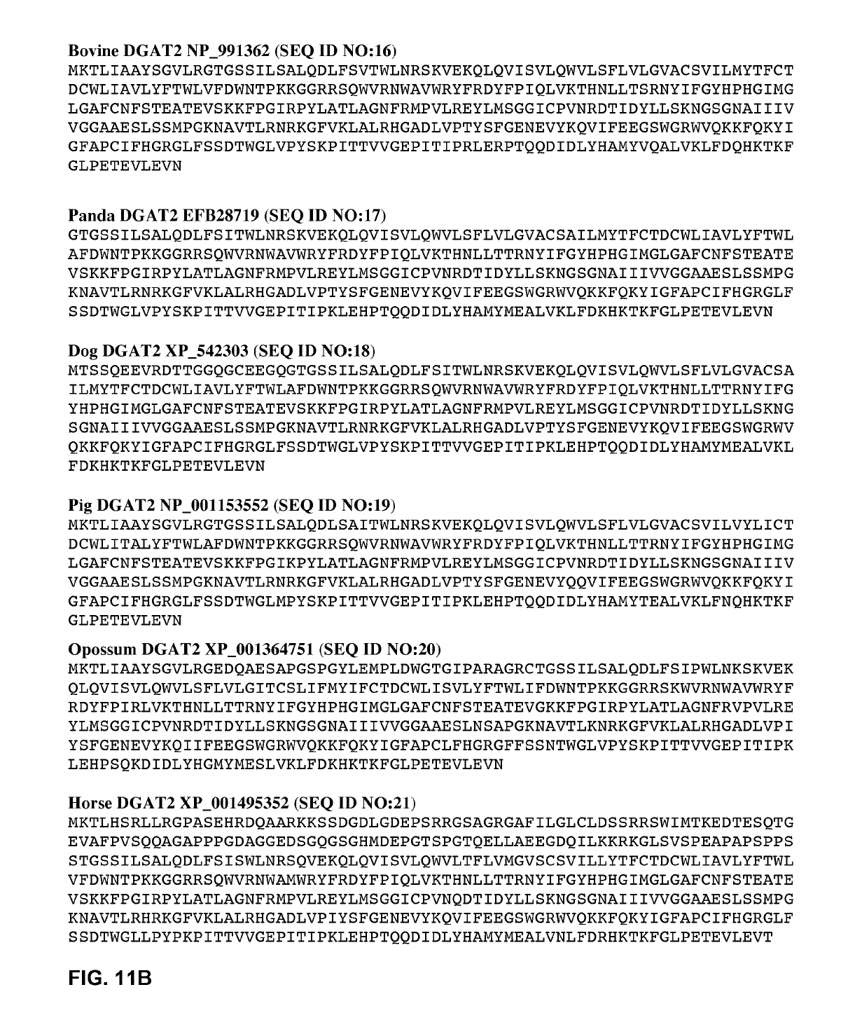

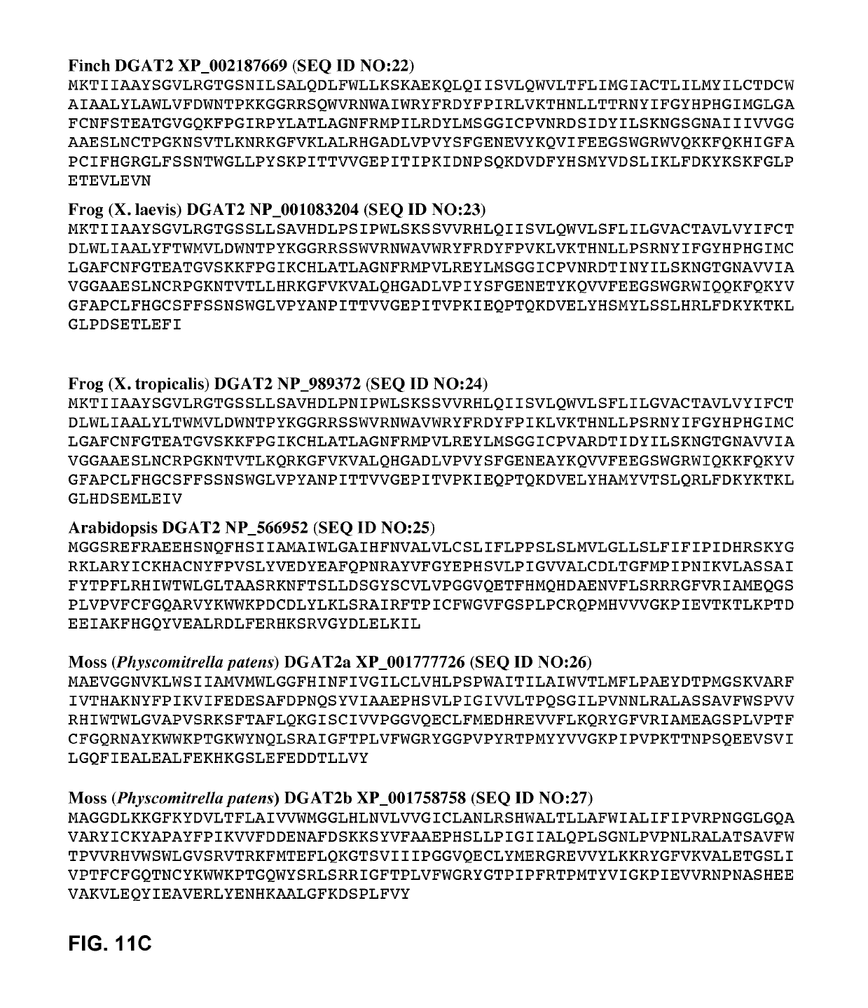

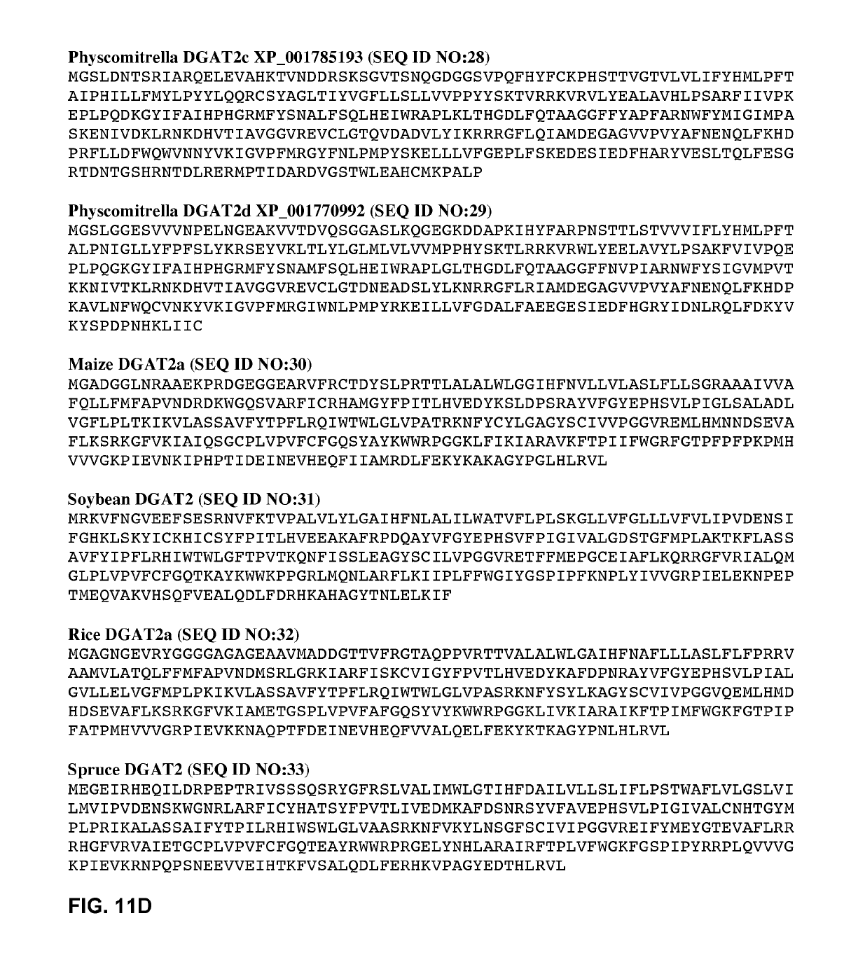

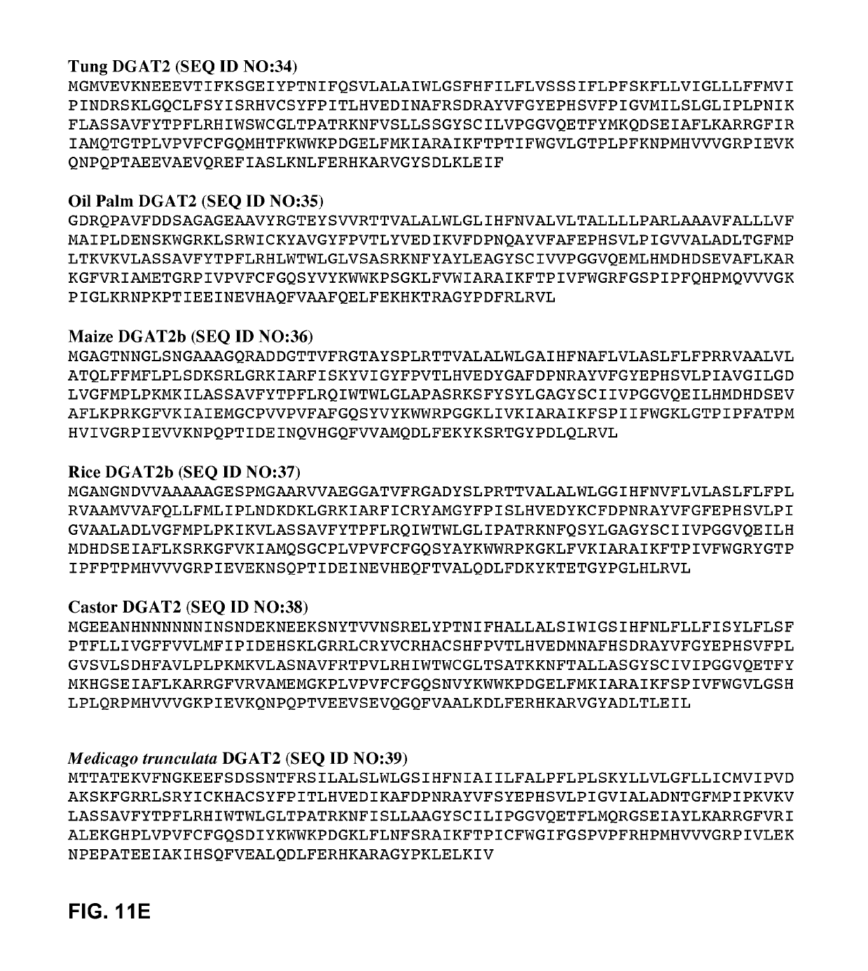

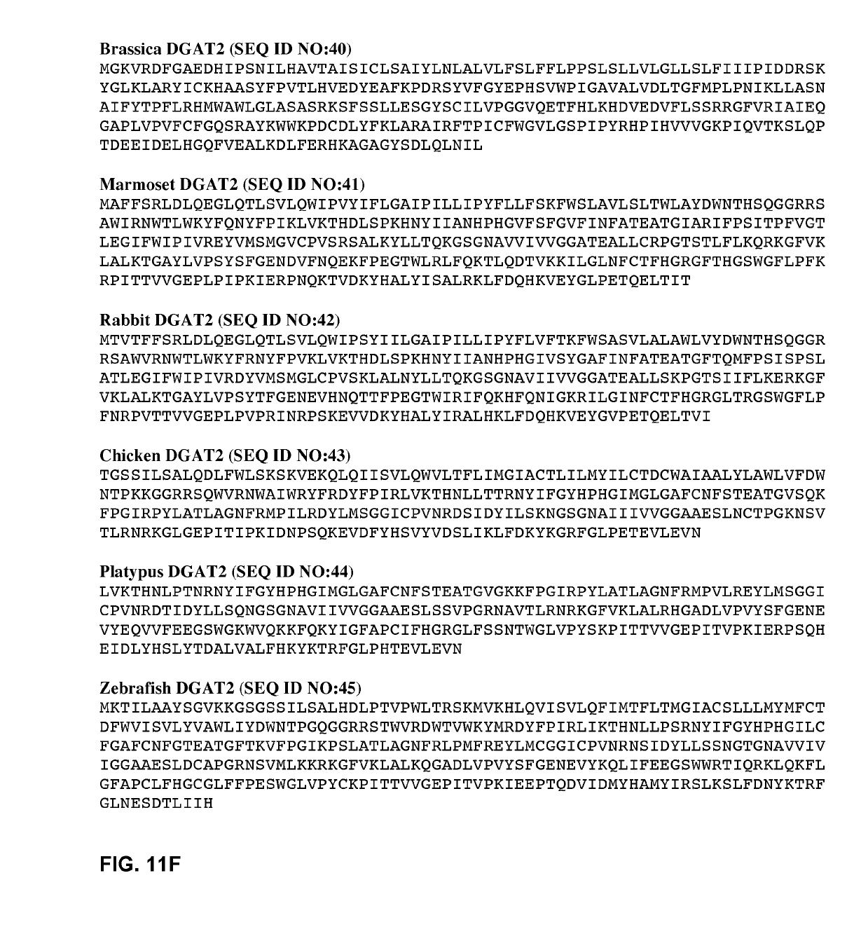

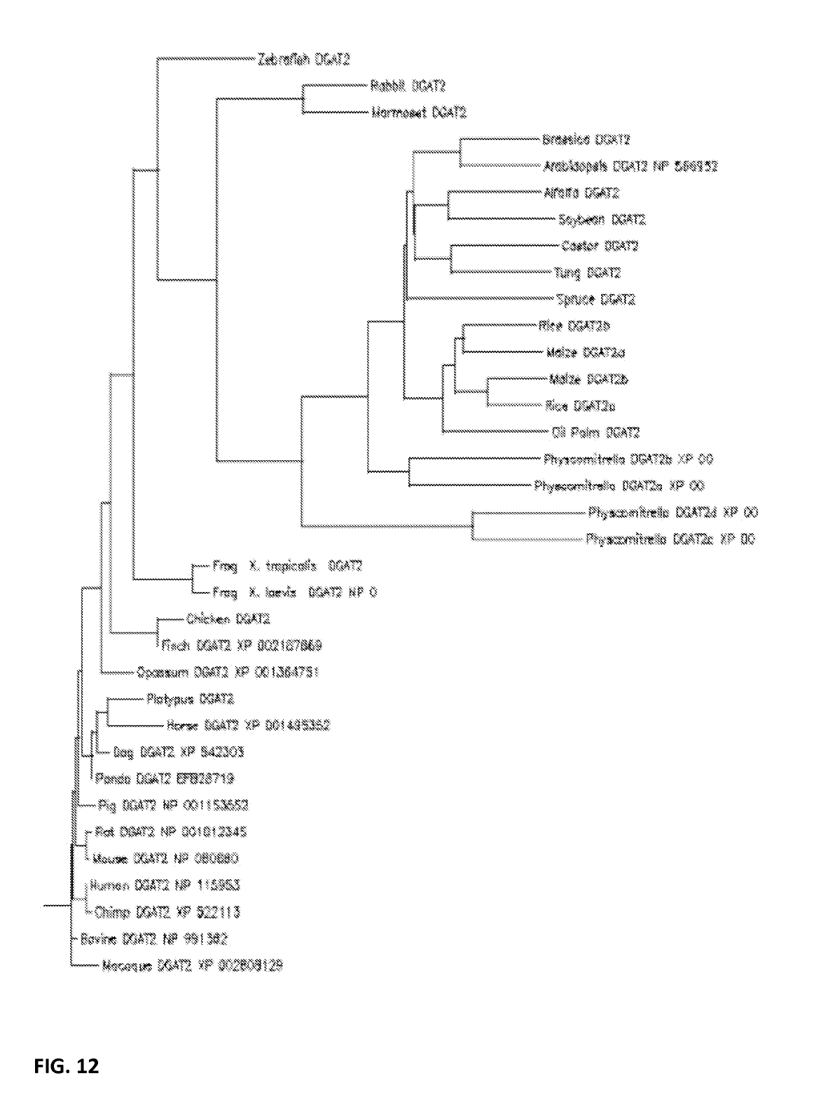

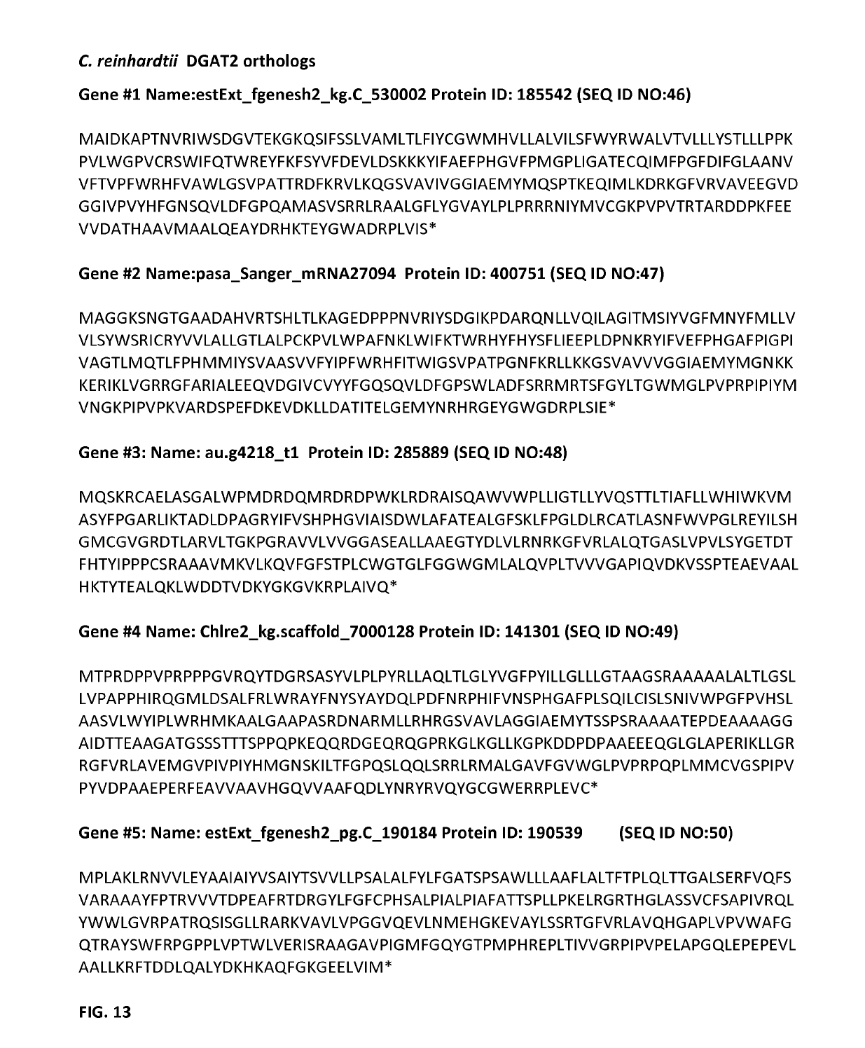

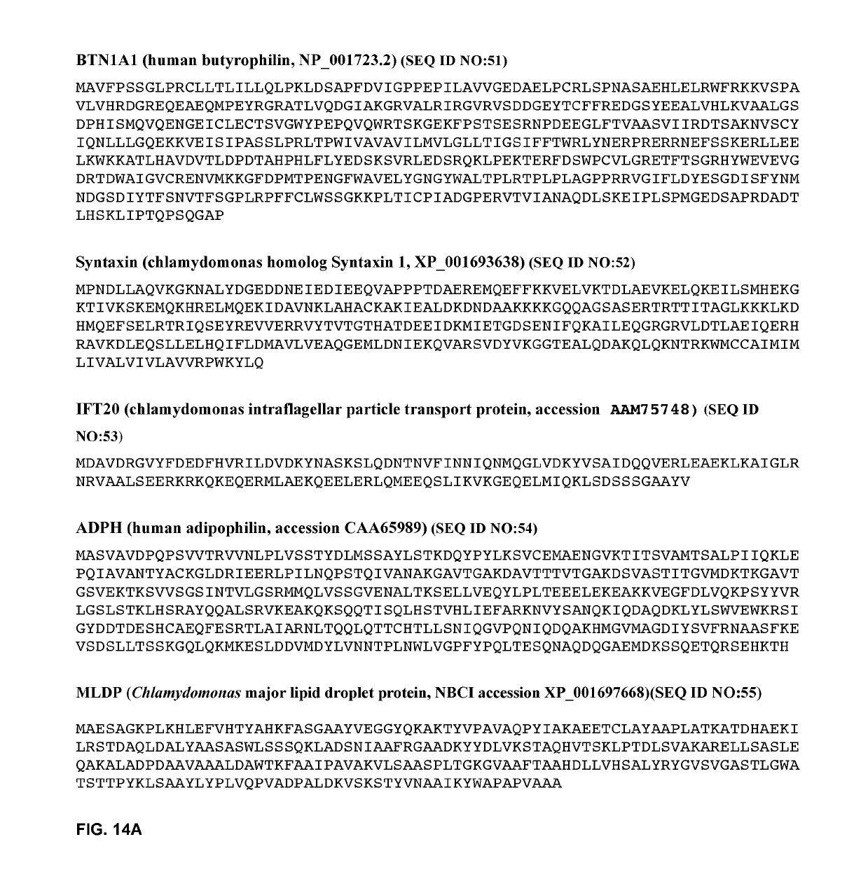

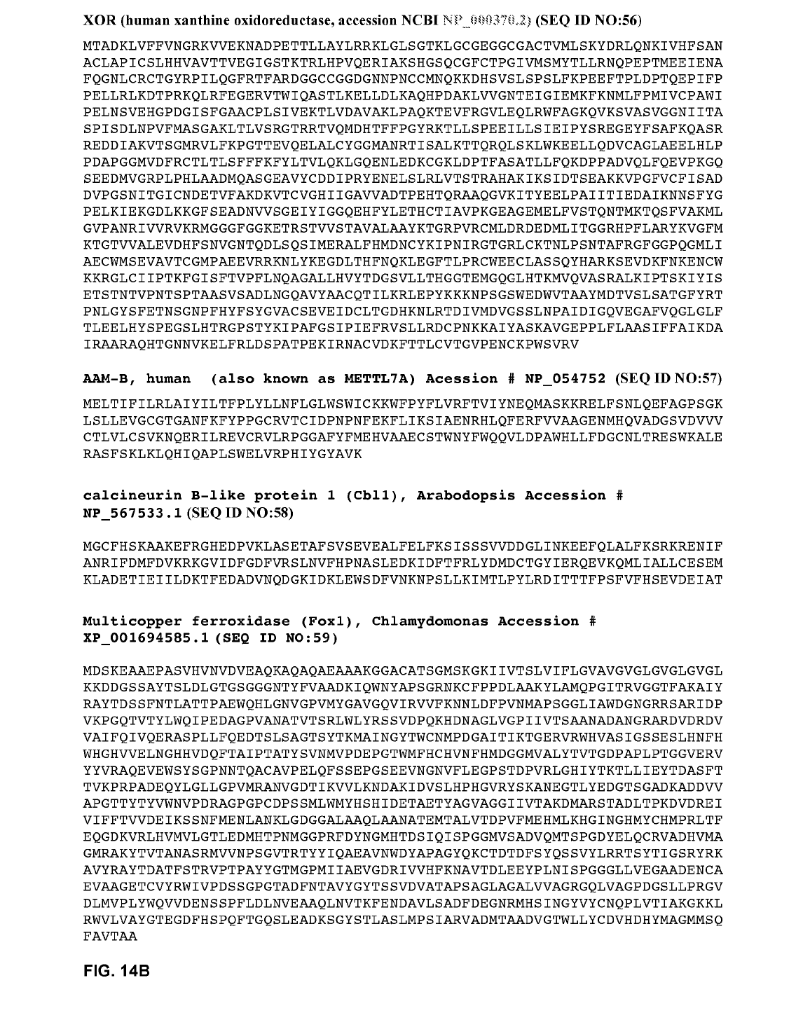

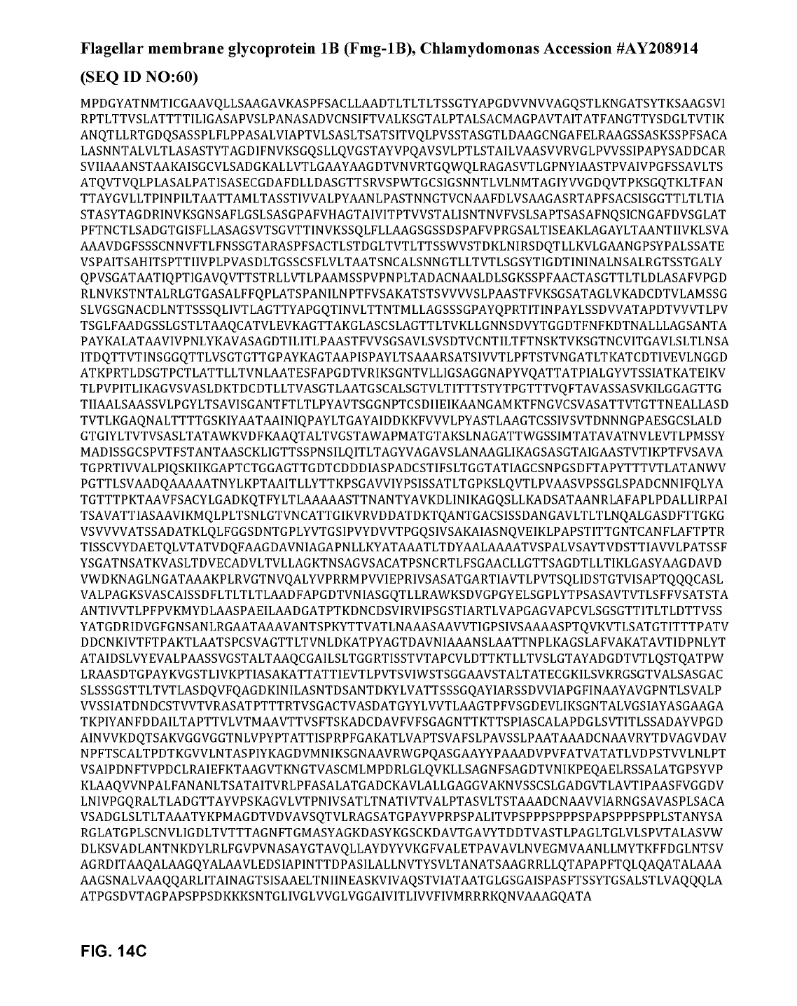

In some embodiments, the vectors of the present invention comprise one of the promoters depicted in FIGS. 9A-9C.

In one aspect, the present invention provides a cell comprising an exogenous promoter having the nucleotide sequence selected from the group consisting of SEQ ID NOs:1 to 10.

In one aspect, the present invention provides a vector comprising a promoter having the nucleotide sequence selected from the group consisting of SEQ ID NOs:1 to 10.

In some embodiments, the vectors of the present invention are used to transfect a host cell using methods known in the art and described herein. Vectors pJR38 and pJR40 are described at Neupert et al., J., Plant J 57:1140-1150 (2009) and vector pKS-aph7-lox is described at Berthold et al., Protist 153:401-412 (2002).

In general, the strains of the present invention retain their expression characteristics over many generations. In some embodiments, the expression of the transformed gene is stable for at least 1, 2, 3, 4, 5, 6, 7, 8, 9, 10, 11, or 12 months, or more of growth on solid media.

In some embodiments, and in contrast to other high-expressing Chlamydomonas mutants previously reported, the strains of the present invention that express transgenes at high levels grow healthily and do not evidence any disadvantage relative to their wild type ancestors.

In some embodiments, the expression of the transformed gene in the host cell is stable. By “stable expression” herein is meant that the transformed gene is retained in the host cell for at least 5, 10, 20, 50, 100, 200, 300, 400, or 500 generations, and being transcribed into RNA and/or expresses the protein it encodes. In general, a stable transformed gene is retained in the host cell for at least 5, 10, 15, 20 or 25 days, or 1, 2, 3, 4, 5, 6, 7, 8, 9, 10, 11 months, or up to two years, and being transcribed into RNA and/or expresses the protein it encodes

In some embodiments, and in contrast to other high-expressing Chlamydomonas mutants previously reported, RNAi is functional in the strains provided by the present invention, permitting for silencing of unhealthy genetic activities such as transposons. Furthermore, the high levels of nuclear transgene expression in the strains provided by the present invention enable for efficient engineered RNAi-mediated gene knockdowns, in contrast to the extreme inefficiency of RNAi-mediated gene knockdowns displayed by previously available strains.

### II. Method of Increasing Lipid Content

In one aspect, the present invention provides compositions and methods for the manipulation of one or more genes in one of more metabolic pathways to increase the lipid content in a cell, such as in an alga. The metabolic engineering methods provided by the present invention enable a cell to increase the production of fatty acids and triglycerides without affecting the growth rates of the cell.

Lipids extracted from algae can be subdivided according to polarity: neutral lipids and polar lipids. The major neutral lipids are triglycerides, and free saturated and unsaturated fatty acids. The major polar lipids are acyl lipids, such as glycolipids and phospholipids. A composition comprising lipids and/or hydrocarbons can be described and distinguished by the types and relative amounts of key fatty acids and/or hydrocarbons present in the composition.

By “fatty acids (FAs)” herein is meant a carboxylic acid with a long unbranched aliphatic chain, which is either saturated or unsaturated. Most naturally occurring fatty acids have a chain of four to 28 carbons. Fatty acids are identified herein by a first number that indicates the number of carbon atoms, and a second number that is the number of double bonds, with the option of indicating the position of the double bonds in parenthesis. The carboxylic group is carbon atom 1 and the position of the double bond is specified by the lower numbered carbon atom. For example, linoleic acid can be identified by 18:2 (9, 12).

Algae produce mostly even-numbered straight chain saturated fatty acids (e.g., 12:0, 14:0, 16:0, 18:0, 20:0 and 22:0) with smaller amounts of odd-numbered acids (e.g., 13:0, 15:0, 17:0, 19:0, and 21:0), and some branched chain (iso- and anteiso-) fatty acids. A great variety of unsaturated or polyunsaturated fatty acids are produced by algae, mostly with C₁₂ to C₂₂ carbon chains and 1 to 6 double bonds, mainly in cis configurations.

In some embodiments, the present invention provides compositions and methods for the simultaneous increase of synthesis and reduction of degradation of triglycerides through the expression of DGAT2, inhibition of lipases, starch synthesis and lipid droplets (LD) rearrangement to significantly increase lipid levels in a host cell (e.g. Chlamydomonas) or combination thereof.

### A. DGAT2 Overexpression

In some embodiments, the present invention provides compositions and method for producing a lipid in a cell generated by over-expressing one or more genes in the production pathway of triacylglycerol.

By “triglyceride (TG)”, “triacylglycerol (TAG)”, or “triacylglyceride” herein is meant an ester composed of a glycerol bound to three fatty acids.

The neutral lipids triglycerides (TGs) are highly reduced stores of oxidizable energy found in most eukaryotic cells and almost absent in prokaryotes. Intracellular TGs are accumulated inside cytosolic membrane-bound organelles called lipid droplets. Most TGs are formed by a reaction in which diacylglycerol (DAG) is covalently joined to long chain fatty acyl-CoAs. The enzymatic activities that catalyze this reaction are called DGATs (DiacylGlycerol AcylTransferase). Two DGAT enzymes, DGAT1 and DGAT2, have been identified in fungus, mammals and plants. Each of both enzymatic activities corresponds to a single polypeptide integrally associated with the membrane of the endoplasmic reticulum. DGAT1 and DGAT2 share no sequence homology despite having similar biochemical activities. In mammals, DGAT1 is expressed in most tissues, with the highest expression levels in the small intestine, testis, adipose tissue, mammary gland, and skin. DGAT2 is expressed highly in tissues that accumulate large amounts of TGs, including liver and adipose.

When expressed from recombinant plasmids in transformed cells and studied in cell-free in vitro assays both DGAT1 and DGAT2 have similar biochemical characteristics as DGATs (including affinity for the DAG and acyl-CoA substrates and catalytic potency). However, results of in vivo experiments suggest that DGAT2 is the actual “TG synthase”. DGAT2 encodes an acyl-CoA:diacylglycerol acyltransferase that catalyze the final step of TG biosynthesis. DGAT2 is an integral membrane protein that resides in the endoplasmic reticulum (ER) and the lipid droplet (LD). Mice lacking both alleles of DGAT2 are almost deprived of TGs, are born small and die soon after birth. In contrast, mice lacking DGAT1 are viable and have only small reductions in tissue TG levels and normal plasma TG levels. Over-expression experiments support the conclusion that DGAT2 is the main TG synthase and reveal that its function is a limiting factor in TG accumulation. Transgenic mice with 2 or 3.5 times higher expression levels of DGAT2 in their livers accumulated 5 and 18 times more liver TGs, respectively. In contrast mice with 90 times higher expression of DGAT1 in their livers accumulated only 3 times more liver TGs.

Work in organisms other than mice is somewhat lagging behind, but published findings suggest that the picture outlined by the experiments in mice holds elsewhere. Flowering plants have genes encoding DGAT1 and DGAT2 enzymes, of which DGAT2 has the highest expression levels in oil-accumulating tissues like developing seeds. Mutant Arabidopsis thaliana with a disrupted DGAT1 gene still synthesizes TGs. Baker's yeasts accumulate triglycerides and have a DGAT2 but not a DGAT1 gene. Mild overexpression of yeast DGAT2 in yeast leads to a 2-fold increase in TGs accumulation.

Wild-type C. reinhardtii is capable of accumulating up to 50% of its dry weight in TGs, but only when deprived of essential nutrients and exhibiting nitrogen stress related responses. Wang et al., Eukaryotic Cell (2009) 8: 1856-1868, and Li et al. Metab Eng (2010). doi:10.1016/j.ymben.2010.02.002.

In some embodiments, the method comprises the over-expression of DGAT2 to increase triglyceride synthesis in the host cells, such as C. reinhardtii. In some embodiments, the expression level of DGAT2 is optimized to increase FA and TG synthesis and accumulation. In some embodiments the Chlamydomonas cells are UVM4 or UVM11

In some embodiments, the genetically engineered cells have normal growth rates and produce 10%, 20%, 30%, 40%, 50%, 60%, 70%, 80%, 90%, or 100% or up to 3000% more total TG than unengineered cells.

In some embodiments, the present invention provides the Chlamydomonas orthologs for DGAT2 (SEQ ID NO:46-50).

In some embodiments, the DGAT2 gene of the present invention is selected from the group consisting of: a human DGAT2 gene, a plant DGAT2 gene, and a Chlamydomonas DGAT2 gene, or a homologue thereof. In some embodiments, the DGAT2 gene has an amino acid sequence that is at least 70%, 75%, 80%, 85%, 90%, 95%, 96%, 97%, 98%, 99% identical to an amino acid sequence selected from the group consisting of SEQ ID NOs: 11-50. In some embodiments, the DGAT2 gene has an amino acid sequence selected from the group consisting of SEQ ID NOs: 11-50.

In some embodiments, the DGAT2 gene used in the present invention is one of the follows where in the parenthesis are the names of the organisms followed by the GenBank accession numbers: At3g51520 (A. thaliana, AAK32844), CeDGAT2 (C. elegans, NP_{—}507469), DGA1 (S. cerevisiae, NP_{—}014888), HsDGAT2 (H. sapiens, NP_{—}115953), HsMGAT2 (H. sapiens, Q35YC2), MmDGAT2 (M. musculus, NM_{—}026384), MmMGAT2 (M. musculus NP_{—}803231), MrDGAT2A (M. ramanniana, Q96UY2), MrDGAT2B (M. ramanniana, Q96UY1), OlDGAT2A (O. lucimarinus, number XP_{—}001419156), OlDGAT2B (O. lucimarinus, XP_{—}001421576), OlDGAT2C (O. lucimarinus, XP_{—}001421075), OtDGAT2A (O. tauri, CAL54993), OtDGAT2B (O. tauri, CAL58088), OtDGAT2C (O. tauri, CAL56438), PpDGAT2A (P. patens, XP_{—}001758758), PpDGAT2B (P. patens, XP_{—}001777726), RcDGAT2 (R. communis, AAY16324), and VfDGAT2 (V. fordii, ABC94473).

DGAT2 and its substrates are localized to the endoplasmic reticulum so this gene needs to be expressed from the nucleus to be active. Therefore, expression from the chloroplast, the basis of algae transgenic expression strategies in other algae biotechnology/biofuel companies, generally is not an option for DGAT2-mediated increase of oil synthesis.

In some embodiments, increasing Chlamydomonas' DGAT2 levels of expression and/or expression of heterologous DGAT2 genes leads to higher accumulation of TAGs.

In some embodiments, an inducible promoter provided herein is used to selectively express DGAT2 at appropriate times and conditions (for example, in order to rapidly grow a high density starter culture, DGAT2 expression is kept off). In general, growth rates is not heavily reduced by overexpressing DGAT2, in contrast to triggering oil accumulation using the standard method of nitrogen-starvation, because nitrogen stress also triggers a wide array of general cellular responses (such as interruption of new protein synthesis) unrelated to carbon flux into cell mass and oil.

### B. Inhibition of Starch Synthesis Pathways

In some embodiments, the method of increasing TAG production comprises inhibiting a gene in the starch synthesis pathway in a cell. The methods of the present invention reduce carbon flux into starches and, indirectly, increase carbon flux into triglyceride synthesis by reducing the expression of genes required for starch synthesis.

Starch synthesis is one of major competitors of triglyceride synthesis for carbon flux. In starch synthesis, cells divert 3-phosphoglycerate, an intermediate in the Calvin cycle, to form glucose-6-phosphate, which is then stored as starches (high molecular weight, branched sugar polymers primarily consisting of amylopectin and amylose). Starch synthesis machinery converts glucose-6-phosphate to glucose-1-phosphate, and then in the first committed step converts glucose-1-phosphate to ADP-glucose, the sole, high-energy building block for starches.

There are two genes required for ADP-glucose synthesis and subsequent starch synthesis in Chlamydomonas. STA1 and STA6 are homologous to 53 kDa regulatory and 50 kDa catalytic subunits, respectively, of ADP-glucose-phosphorylase (AGPase) subunits in higher plants. Sta1-mutants reduce starch levels to <5% of wild-type levels, and stab-mutants reduce starch levels to <0.01% of wild-type levels.

In some embodiments, STA6 and/or STA1 genes (Table 1) are knocked out or knocked down by methods known in the art, such as by RNAi (FIG. 4). Reduced expression is advantageous to mutational inactivation because residual enzymatic activity can allow the accumulation of small amounts of starch, which is needed for cell growth. Hence, strains with STA6 expression reduced by RNAi have higher growth rates than strains with a complete loss of STA6 function caused by mutation (FIG. 4). In some embodiments, inducible switches for the expression of the RNAi constructs targeting STA1 or STA6 are used for expression at optimal times and conditions. In some embodiments, promoters of different strength control the expression of the RNAi constructs targeting STA1 or STA6 for expression at different levels.

TABLE 1

| | | | Protein | |
|---|---|---|---|---|
| protein | gene | name | ID | Location |
| ADP glucose | STA1 | SKA_estExt_fgene | 205913 | Chlre3/scaffold_2:92 |
| phopshorylase large | | sh2_pg.C_20112 | | 2680-929270 |
| regulatory subunit | | | | |
| ADP glucose | STA6 | estExt_gwp_1W.C_40184 | 136037 | Chlre3/scaffold_14:4 |
| phosphorylase small | | | | 17754-422496 |
| catalytic subunit | | | | |

### III. Active Secretion

In some embodiments, the present invention provides a method for producing a lipid, comprising: culturing a genetically engineered cell; and producing a lipid (e.g. a triglyceride) secreted from the genetically engineered cell. In some embodiments, the lipid is secreted in the form of a lipid droplet. In some embodiments, the lipid is secreted in the form of a fat globule. In some embodiments, the lipid is secreted within a flagellum, wherein the flagellum is shed or otherwise detached in part or wholly from the cells into the media.

The most challenging and costly steps in conventional algae-to-oil are efficiently harvesting cells from large growth volumes and then extracting lipids from the cell. Typically, cells are harvested by centrifugation to separate them from the growth media. Oils are then either physically or chemically extracted. This processing pipeline processes cells in “batches”. Centrifugation is an energy intensive process requiring expensive equipment. For example, it was estimated that conventional centrifugation of cells to harvest costs greater than $500/tonne of cells or ˜$150/bbl oil pre-extraction. These costs particularly reduce the economic viability of large-volume open pond growths, which tend to produce cultures with lower cell densities (and thus require larger volumes to be processed to produce a given volume of oil).

The present invention solve the harvesting/extraction bottleneck in algal biofuel production by sidestepping it with engineered biological oil secretion processes.

In one aspect, the present invention provides a lipid droplet secretion system. The system

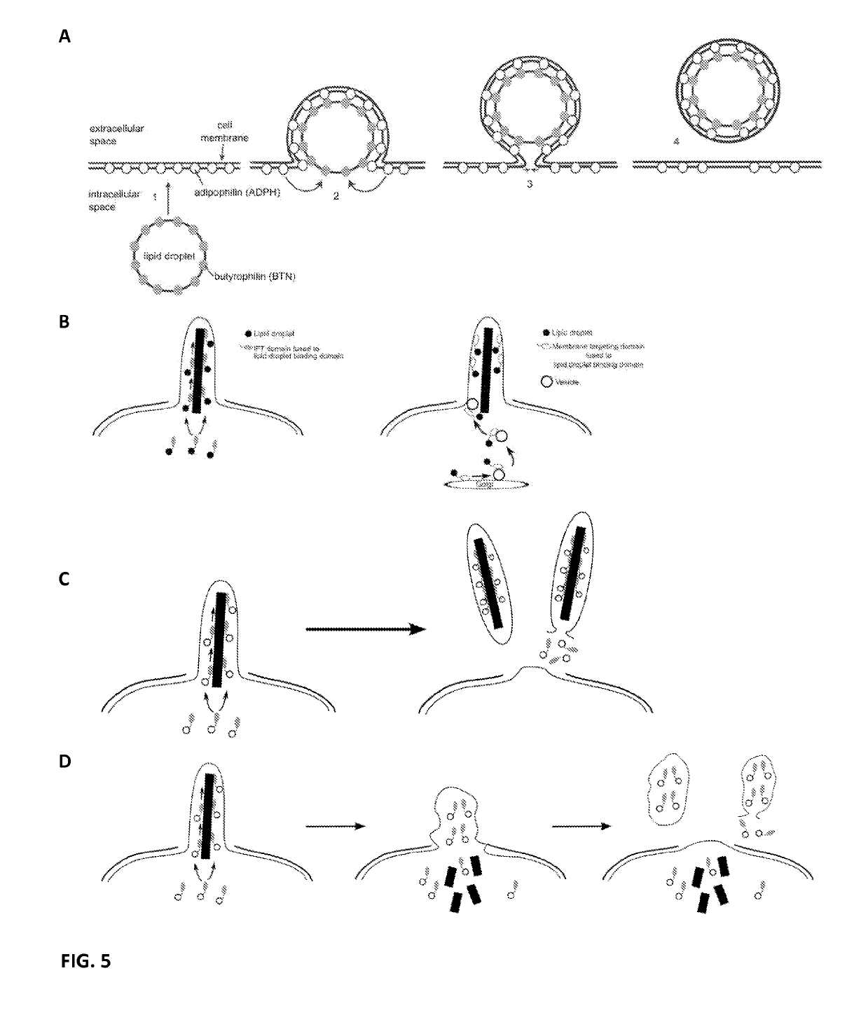

performs two tasks: 1) recruit cytoplasmic LDs to the plasma membrane, to the flagellar membrane or to the flagellar lumen, and 2) secrete the LDs (by themselves, in lipid vesicles, or enclosed in plasma membrane or flagellar membrane, or bound to plasma membrane or flagellar membrane) into the extracellular space (FIG. 5). Released lipids, less dense than water, can float to the surface of the aqueous growth media, and can be skimmed off of the surface. The advantage of this process over other projects that directly produce small hydrocarbon fuels is a matter of productivity: sequestered lipids can be produced in the cell and accumulated in the media without becoming toxic to the cells. Because the oil secretion production system provided herein naturally sequesters hydrophobic, oily compounds with a lipid bilayer, and expels them from the cell, the cell can produce very large amounts without injuring the cell, unlike, for example, the direct production of ethanol, butanol, or smaller hydrocarbon-like fuel compounds that are toxic to the cells at even moderately high concentrations. This allows higher production rate of high-energy compounds, at the small cost of additional downstream refining and processing, which are established at production-level scales.

In another aspect, the present invention provides a lipid droplet secretion system comprising: (i) a first domain (e.g., proteins or protein fragments) that binds to the plasma membrane or flagellar membrane (the membrane-binding domain), and (ii) a second domain (e.g., proteins or protein fragments) that binds to the lipid droplet (the lipid droplet-binding domain). The membrane-binding domain interacts with the lipid droplet-targeting domain by non-covalent interactions between the domains, or by non-covalent interactions between additional domains fused to the membrane- and lipid droplet-binding domains, or by covalent fusion of the membrane- and lipid-binding domains. After recruitment of lipid droplets to the plasma membrane, the flagellar membrane, or into (or near) the flagella, the lipids are released in two general ways. In some embodiments, the expressed proteins cause the lipid droplet to be recruited to the plasma or flagellar membrane and then enveloped by the membrane. The enveloped lipid droplet is then pinched off in manner similar to milkfat secretion, leading to the separation of the bilayer-enclosed LD from the plasma or flagellar membrane. In some embodiments, the expressed proteins cause the lipid droplet to be recruited into the flagella or near the base of the flagella via intraflagellar transport proteins (or proteins that generally shuttle into the flagella), or to the flagellar membrane. The flagellar-associated lipid droplets then are released into the media during flagellar release (deflagellation) or flagellar resorpotion. The released lipids may be in the form of lipid droplets, lipid vesicles, or enclosed within part or all of the flagella or flagellar membrane.

This invention provides every possible combination of (i) plasma membrane or the flagellar membrane targeting domains, with (ii) lipid droplets targeting domains, adding expression tags such GFP and hexaHistidine in some cases. The parts will combined covalently or co-expressed together with interaction domains, The DNA sequence of each of the possible combinations is the combination of the sequences of the different plasma membrane-, flagellar membrane-, and lipid droplet—targeting proteins and domains described elsewhere in the patent.

### A. Mammary Cells LDs (Milkfat) Secretion Systems

In some embodiments, the lipid droplet secretion systems of the present invention employs system components from the system that mammary cells use to secrete intracellular LDs (milkfat) into milk.

Milkfat consists of over 98% TAGs in the form of milkfat globules (MFGs). These globules consist of LDs (TGs surrounded by a monolayer phospholipid membrane) surrounded by a lipid bilayer derived from the plasma membrane. Cells form and secrete MFGs primarily by enveloping LDs with the apical plasma membrane. A number of proteins are essential for secretion. See McManaman, J. L., et al., J Mammary Gland Biol Neoplasia, 2007, 12(4): 259-68.

A combination of biochemical and histological studies suggest that at least two proteins mediate docking of LDs to the apical plasma membrane surface and thus secretion, butryophillin (BTN) and xanthine ornithoreductase (XOR). BTN, a type I single pass transmembrane protein, localizes to the plasma membrane, and XOR is cytoplasmic. A third protein, adipophilin (ADPH), probably acts as the LD-specific protein anchor. ADPH resides on the LD surface. XOR (+/−) mice or BTN (−/−) mice show increased accumulation of larger LDs at the plasma membrane.

An N-terminal deletion of ADPH mildly reduces (˜10%) the fat content of secreted milk. ADPH protein levels and mRNA levels correlate with lipid accumulation, and mouse mammary epithelial cells express high levels of ADPH. Detergent extracts of MFG membrane preparations yield protein complexes with all three proteins, ADPH, XOR, BTN, and co-localization studies show these three proteins co-localize on the apical membrane surface at sites of lipid secretion. Cross-linking studies suggest BTN and XOR are in close proximity. A GST-C-terminal region of mouse BTN fusion also binds to XOR from cell lysates, consistent with interactions between XOR and BTN. These data do not definitely show but strongly suggest that these proteins are directly involved in LD docking and budding.

It is less known about the molecular details of actual secretion. A popular model proposes the following mechanism. First, the LD binds, via ADPH, to BTN monomers in the plasma membrane. This binding induces formation of higher order oligomers of BTN in the plasma membrane; ADPH/BTN binding and BTN oligomerization provide forces to curve the membrane around the LD. XOR recruitment to BTN oligomers stabilizes the curvature of the plasma membrane. In this model, the continued wrapping of the plasma membrane around the LD eventually forms a neck in the bilayer that spontaneously pinching shut, leading to separation of the bilayer-enclosed LD from the apical plasma membrane.

B. Secretion Systems that Involve the Shedding of Algae Flagella

In another aspect, the present invention provides methods to secrete intracellular oils by targeting lipid droplets into or near the flagella. Many green algae such as Chlamydomonas reinhardtii have motile flagella. Algae flagella are whip-like appendages used for locomotion and for pair formation during mating. The flagella are structurally complex, containing more than 250 types of proteins. Each flagellum contains an axoneme, or cylinder, with nine outer pairs of microtubules surrounding two central microtubules. The axoneme is surrounded by the flagellar membrane, which is an extension of the cellular plasma membrane.

The flagella are not enclosed by the cellulose cell wall that encases the algae cell. They emerge through holes in the cell wall called collars. A contribution of the present invention is to note that these two holes can serve as sites from where lipid droplets can be secreted from the cell interior into the media using an engineered secretion system.

Algae flagella are dynamic structures: both their components are turned over continuously and the entire structure disappears and reappears in different conditions. Flagellar turnover occurs at the tip; both disassembly of old structures and assembly of new ones are localized at the far end of the flagella. Components are moved in and out by particles that travel on microtubules via the process of intraflagellar transport (IFT). Old components are retrieved from the flagella and shipped back into the cell body via retrograde intraflagellar transport, and new components are carried to the tip via anterograde flagellar transport.

Flagellar loss can happen by deflagellation or resorption. When deflagellation occurs, the flagellar stem is severed proximal to the cell body and the entire structure is shed from the cell. During resorption, assembly at the tip stops and continuing disassembly leads to flagellar shrinkage and eventual disappearance. Deflagellation is triggered by a variety of stimuli, most of which are associated with unsuitable environmental conditions or “stress” situations such as low pH. When suitable conditions return, the cells regrow their flagella. Resorption happens during every cell division. When cells enter mitosis, their flagella shrink and disappear, and after mitosis the cells regrow their flagella. While major protein components of the flagella (e.g., the axoneme) are disassembled and shipped back into the cell, it is not clear if the flagellar membrane is disassembled and its constituents re-used by the cell, or severed, or disassembled by some other means. Resorption also happens when the cells are exposed to various poisons, such as sodium pyrophosphate (NaPPi) or isobutylmethylxanthine (IBMX). In those cases resorption is presumed to function to prevent the exposed membrane surface of the flagella from contacting the poisons.

In one aspect, the present invention provides methods and compositions to exploit both flagellar severance and flagellar resorption. In some embodiments, a secretion system is engineer that first loads the flagella with the cargo to be secreted, which is then released into the media accompanying flagellar remnants. During deflagellation the entire flagellar structure is released into the media and it is all but certain that a cargo that had been previously delivered to the flagella would be shed as well. The transportation of lipid droplets into the flagella, such as by using intraflagellar transport proteins like IFT20 that bind to protein structures within the flagella, or by using flagellar membrane proteins like Fmg1B that end up in the flagellar membrane, followed by deflagellation constitutes one mechanism for secreting lipid droplets from inside cells into the media. (FIG. 5D).

Release of flagellar components can also occur during resorption. For example, it is unclear what the fate of the flagellar membrane is during resorption. There are no known mechanisms that remove membrane from the flagella during shrinkage, such as membrane retrieval by endocytotic mechanisms. It is likely that the excess membrane of the shrinking flagella is simply released from the cell.

Secretion of lipid droplets may also occur by simple transport of lipid droplets near the base of the flagella. In cases where the cells do not possess fully-formed flagella (e.g., after a flagellar resorption event), lipid droplets may still be secreted by transport near the base of the flagella because at that location there is a hole in the cell wall for the flagella to extend from the cell. Lipids droplets, after being transported to the region near where the flagella will eventually fully form, may be secreted by a mechanism similar to milkfat secretion (as described above), where due in part or in whole to intraflagellar transport forces, the lipid droplets are pressed against and enveloped by the incipient flagellar membrane or adjacent cell membrane near the hole in the cell wall and, after membrane pinching, are secreted from the cell. Alternately, any transient pores or holes formed in the incipient may allow lipid droplets in the vicinity to exit (i.e., be secreted by) the cell. Targeting lipid droplets near the flagellar, either by targeting lipid droplets to the flagellar membrane such as by tethering them to Fmg-1B, or by delivering lipid droplets toward the base of the flagella, such as by tethering them to IFT20/intraflagellar transport machinery components, should result in the release to the media of the lipid droplets bound surrounded by flagellar or cell membrane, or result in the release of lipid droplets.

Intraflagellar transport (IFT) is carried out by IFT particles. IFT particles are complexes of at least 17 different polypeptides, which are associated with the flagellar membrane. The IFT particles move from the cytoplasm out to the flagellum and travel to the tip along the outer doublet microtubules, bringing any associated proteins along with them. At the tip they unload their cargo and are available to pick up disassembled components. The particles loaded with old components then make the trip back from the tip to the base of the flagella.

One of the IFT proteins, IFT20, is membrane associated and traffics between the Golgi apparatus and the flagellum. IFT20 with GFP fused at its C-terminus functions normally. In some embodiments, IFT20 is fused to a lipid-droplet binding domain at its C-terminus to tether LDs to IFT particles. IFT particles tethered to LDs should carry LDs into the flagella.

The flagellar lumen and the cytoplasm are separated by a region called the transition zone. The space between the flagellar membrane and the axoneme at the transition is filled with protein structures that tether the axoneme to the membrane. This structure constitutes a “gate” that restricts the movement of particles in and out of the flagella.

The dense protein network in the transition zone limits the size of the lipid droplets that can be carried into the flagellar lumen by the engineered carrier proteins described in this invention (fusions of IFT20 or FMG1 to LD-binding domains). The size distribution of the lipid droplets is 100-1000 nm in width. The observed width for passing through the transition zone is 10-30 nm, which suggest that only the tiniest lipid droplets, which make up a fraction of the total LD content of the cell, can be carried into unmodified flagella. One way to increase the rate of transport of lipid droplets into the flagellar lumen is to modify the structural components of the transition zone to enlarge the width of the path of transit.

The identity of most protein components of the transition remains unclear. A group of proteins collectively referred as NPHPs (from “nephronophthisis”, a group of human diseases caused by ciliary disfunction arising from mutations in the NPHP genes) has been shown to localize near the transition zone in human cells. Recently, one of these proteins, Nphp6/Cep290, has been shown to be a structural component of the transition zone in the flagella of Chlamydomonas reinhardtii. Nphp6/Cep290 connects the axoneme to the plasma membrane at the base of the flagella. The Nphp6/Cep290 appears to act as a physical barrier that keeps the proteins in the flagellar lumen from mixing with the proteins in the cytoplasm. Mutants carrying loss-of-function alleles of Nphp6/Cep290 show a disorganized architecture at the transition zone and have large changes in the protein composition of the flagellar lumen.

In these transition zone mutants much larger LDs could cross into the flagellar lumen from the cytosol without being sterically excluded by the proteins in the transition zone. This could increase the number of LDs and total amount of lipid transported into the flagella using the engineered flagellar loading systems proposed in this invention. Similarly, disruption of the transition zone by any other means, including but not limited to expressing dominant negative variants of transition zone proteins and downregulating the transition zone proteins by RNAi, could similar accomplish the goal of enabling larger LDs to be carried into the flagellar lumen. In some embodiments, the engineered LD secretion systems based on loading the flagella with LDs have higher secretion yields in mutant Chlamydomonas strains carrying nphp6/cep290 loss-of-function alleles or any other mutation or alteration that leads to a widening of the opening at the base of the flagella.

The size distribution of the LDs is altered when one of several genes is down-regulated by RNAi (Guo et al., Nature, 2008, 453(7195):657-61). One class of genes caused the lipid droplets to be bigger when down-regulated. This class (“Class III”) included ARF1, which encodes a small GTP-binding protein involve in vesicular trafficking in the Golgi apparatus. The fact that down-regulation of ARF1 causes the LDs to become larger suggests that LDs are being constantly turned over by shedding small vesicles containing TGs. The small vesicles could also be transported into the flagella by the flagellar secretion systems described herein. Guo et al., Nature, 2008, 453(7195):657-61. Without being bound by any particular theory, it is noted that overexpression of ARF1 (or other genes of its class) is likely have the opposite effect: it may increase the number of small vesicles leaving the LDs, thus causing the LD size to decrease. Overexpression of ARF1 (or other genes of its class) could also increase the number of small vesicles full with TGs. Both things, reducing the size of the LDs and increasing the number of small vesicles, can lead to increased rates of loading of LDs into the flagellar lumen.

Another class of genes that leads to reduced LD size when downregulated by RNAi (“Class V” genes) includes genes that encode enzymes that synthesize membrane lipids. In Drosophila two genes encoding enzymes involved in the synthesis of phosphatidylcholine were found in this class of genes. At least one of these enzymes localizes to the surface of the LDs. It is likely that their presence on the surface of the LDs leads to an appropriate supply of new membrane, which is needed to generate smaller LDs from a larger one. Thus, downregulation of these genes by RNAi likely blocks LD fragmentation, leading to larger LD size. Chlamydomonas lacks phosphatydilcholine, but has many enzymes that synthesize other membrane lipids (particularly the betaine lipid diacylclyceryltrimethylhomoserine or DGTS) that could fulfill an analogous role on the membrane of the LDs. Overexpressing these enzymes involved in membrane synthesis is likely to have the opposite effect: it may increase the rate of generation of smaller LDs, by providing a larger supply of LD membrane. As above, smaller LDs can lead to increased rates of loading of LDs into the flagellar lumen.

Another class of genes (“Class II” genes), when downregulated by RNAi, leads to smaller LDs (as opposed to the classes mentioned above, that when downregulated caused larger LDs). This class included genes involved in a diverse spectrum of biological processes, including subunits of the COPS signalosome complex, dynein, and RNA polymerase II subunits. Downregulating expression of homologous genes in this class is another method to generate smaller LDs, which, as described above, may lead to increased rates of loading of LDs into the flagellar lumen and thus increased amounts of secreted LDs.

### C. Lipid Droplet Secretion Systems

In one aspect, the present invention provides a cell transformed with and stably expresses one or more gene selected from the group consisting of: BTN, Syntaxin (an integral membrane protein involved in exocitosis in all eukaryotes), FMG1-B (a flagellar integral membrane protein of, e.g. C. reinhardtii), IFT20, ADPH, MLDP (a droplet membrane associated protein of, e.g. C. reinhardii lipid), Fox1 (a membrane protein of, e.g. C. reinhardtii), Cbl1 (a membrane-associated protein of, e.g. Arabadopsis thaliana) and AAM-B (a mammalian lipid droplet membrane associated protein), or a fragment thereof.

In some embodiments, the cell is a mammalian cell, a plant cell, a yeast cell, or an alga cell. In some embodiments, the alga is a Chlamydomonas, such as C. reinhardtii UVM4 or UVM11.

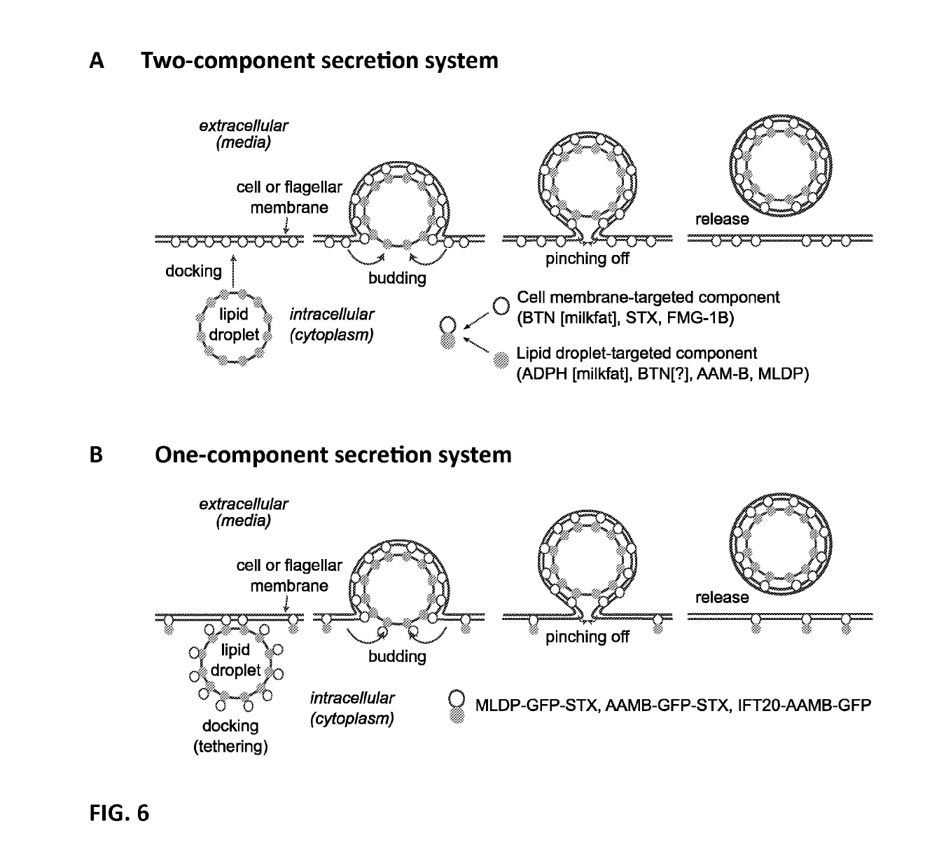

In some embodiments, the lipid droplet secretion system is a one part system with only one polypeptide is used. The polypeptide is a fusion of (i) a LD-targeting domain and (ii) a plasma membrane-targeting domain or flagellar membrane-targeting domain or flagellar-targeting domain. Membrane targeting domains are selected from proteins BTN, Syntaxin, FMG1-B, Fox1, Cbl1, or IFT20. LD targeting domains are selected from BTN, ADPH, MLDP, or AAM-B. See FIG. 6.

In some embodiments, the lipid droplet secretion system is a two-part system wherein two proteins are used and are carried with different vectors. One part comprises a protein attached to the plasma membrane or flagellar membrane-targeting domain or flagellar-targeting domain, which is BTN, Syntaxin, FMG1-B, Fox1, Cbl1, or IFT20. The other part comprises a protein attached to the LD, which is BTN, ADPH, MLDP, or AAM-B. See FIG. 6. In the two part system, each of these two proteins also contain an interaction domain that mediates binding between the membrane-attached protein part and the lipid droplet-attached protein part. In some embodiments, a pair of interaction domains consists of BTN and ADPH themselves. In other embodiments, the pair of interaction domains consists of a PDZ domain and its cognate target sequence. In other embodiments, the pair of interaction domains consists of an SH3 domain and its cognate target sequence. In other embodiments, the pair of interaction domains consists of a leucine zipper motif and its cognate target sequence.

An exemplary BTN has sequence of SEQ ID NO: 51.

An exemplary Syntaxin has sequence of SEQ ID NO: 52.

An exemplary IFT20 has the sequence of SEQ ID NO: 53.

An exemplary ADPH has the sequence of SEQ ID NO: 54.

MLDP (major lipid droplet protein, NCBI accession number XP_{—}001697668) (SEQ ID NO: 55) is a protein identified in C. reinhardtii. It appears to be the most abundant protein in the lipid droplet of secreted by C. reinhardtii. Moellering and Benning, Eukaryotic Cell (2010) 9(1):97-106.

An exemplary XOR (cytosolic) has the sequence of SEQ ID NO: 56.

AAM-B peptide targets heterologous proteins to lipid droplets in yeast. An exemplary AAM-B peptide comes from the protein sequence of SEQ ID NO: 57.

An exemplary Cbl1 (accession no. NP_{—}974566.1) has the sequence of SEQ ID NO 58.

An exemplary Fox1 (accession no. XP_{—}001694585.1) has the sequence of SEQ ID NO 59.

FMG1-B (flagella membrane glycoprotein 1B, Accession No. AY208914) is a C. reinhardtii protein that appears to function in gliding. An exemplary FMG1B has the sequence of SEQ ID NO:60

In some embodiments, the cell is transformed to express both BTN and adipophilin.

In some embodiments, the cell is transformed to express ADPH, XOR, and BTN. In some embodiments, the proteins are each tagged with small C-terminal, N-terminal, or internal tags for immunoquantification.

The transgenes are driven by constitutive or, inducible promoters, therefore, the expression of secretion machinery is either actively regulated (e.g., actively induced by some method when measured LD levels in cells to be high enough), or is passively regulated by engineered circuits (e.g., self-induced at a certain level of intracellular LD accumulation, cell density, etc.).

### Protocol for Producing the Secreted FA

In one aspect, the present invention provides a genetically engineered cell, the cell secrets a lipid through a non-toxic mechanism, such as the lipid is secreted in the form of a lipid droplet, or in the form of a fat globule. In some embodiments, the lipid comprises a triglyceride.

By “non-toxic secretion mechanism” herein is meant a mechanism where hydrophobic, oily compounds are sequestered with a lipid monolayer and which is later expelled from the cell. Unsequestered hydrocarbon fuels like ethanol, butanol, or smaller hydrocarbon-like fuel compounds are toxic to the cells at higher concentrations. With hydrocarbons sequestered in the lipid monolayer, the cell can produce very large amounts without injuring the cell.

In some embodiments, the cell is an alga stably transformed with one or more genes encoding a protein selected from the group consisting of: BTN, Syntaxin, FMG1-B, IFT20, Cbl1, Fox1, ADPH, MLDP, and AAM-B, or a fragment thereof.

In another aspect, the present invention provides a composition, comprising a fat globule comprising triglyceride surrounded by a lipid monolayer and a lipid bilayer. In some embodiments, the composition further comprises one or more proteins selected from the group consisting of: BTN, Syntaxin, FMG1-B, IFT20, Cbl1, Fox1, ADPH, MLDP, and AAM-B, or a fragment thereof.

In another aspect, the present invention provides a composition, comprising a droplet comprising triglyceride surrounded by a lipid monolayer. In some embodiments, the composition further comprises one or more proteins selected from the group consisting of: BTN, Syntaxin, FMG1-B, IFT20, Cbl1, Fox1, ADPH, MLDP, and AAM-B, or a fragment thereof.

In another aspect, the present invention provides a composition, comprising a vesicle comprising triglyceride surrounded by a lipid bilayer. In some embodiments, the composition further comprises one or more proteins selected from the group consisting of: BTN, Syntaxin, FMG1-B, IFT20, Cbl1, Fox1, ADPH, MLDP, and AAM-B, or a fragment thereof.

### IV. Biosynthesis of Other Products

In another aspect, the present invention provide compositions and methods for the introduction of a non-native biosynthetic pathway for the production of Retinol (Carotenoid) and DHA/EPA (ω-3 Fatty Acids).

### A. Retinol

In yet another aspect, the present invention provides a method for producing retinol or for increasing the production of retinol, comprising: culturing a genetically engineered cell to produce retinol, wherein said cell is transformed with a β-carotene: oxygen 15,15′-monooxygenase gene and a aldehyde NAD(P)H reductase gene.

In some embodiments, the cell is a Chlamydomonas cell, such as C. reinhardtii UVM4 or UVM11.

C. reinhardtii synthesizes a relatively large amount of β-carotene and other carotenoid derivatives, such as lutein, loroxanthin, and the xanthophylls neoxanthin and violaxanthin. Cumulatively, these accumulate to about 1 mg/l of standard medium density culture. The carotenoids serve various functions for the cell, acting as light harvesting and energy transfer chromophores, and serving as potent anti-oxidants that inactivate reactive oxygen metabolites, primarily during photosynthesis. However, the bulk of the carotenoids are found in the eyespot. The carotenoids in the eyespot are embedded in a proteinaceous structure that holds thousands of carotenoid molecules together. The carotenoid structures appear as two layers of globuli within the chloroplast and directly below the plasma membrane region where the photoreceptors localize. The carotenoid globuli function to prevent light from illuminating the back of the photoreceptor patch in the membrane and thus enable the cells to detect the direction where light is coming from. Cells without carotenoid globuli are unable to track the source of light but are otherwise healthy and metabolically unimpaired, suggesting that diverting carotene to other pathways would not compromise any cell function relevant to the uses given to the cells in the projects depicted here.

The carotenoid backbone is made up of eight 5-carbon isoprene units. Isoprene biosynthesis in Chlamydomonas and other plants occurs in the chloroplast, via the non-mevalonate, methyl-erythritol-5-phosphate pathway. Carotenoids made during vegetative growth accumulate in the chloroplast and in the eyespot.

Retinol, the animal form of vitamin A, is a fat-soluble alcohol that can be derived from β-carotene in two steps. First, β-carotene is split into two molecules of retinal (the aldehyde form) after being oxidized in a reaction catalyzed by β-carotene:oxygen 15,15′-monooxygenase (FIG. 9). Then, each retinal can be further reduced to retinol by NADH in a reaction catalyzed by retinol:NAD+ oxidoreductase (FIG. 9).

As mentioned above, algal carotenoid synthesis occurs in the chloroplast lumen. In one embodiments, the present invention provide a method of producing retinol or increasing the production of retinol by expressing β-carotene:oxygen 15,15′-monooxygenase and a retinol:NAD+ oxidoreductases targeted to the thylakoid membranes

In one embodiments, the present invention provide a method of producing retinol by expressing a β-carotene:oxygen 15,15′-monooxygenase targeted to the thylakoid membranes. Reduction of retinal to retinol may occur due to broad-specificity aldehyde NAD(P)H reductases that are basally expressed in the chloroplast.

The enzymes used in the present invention include, but are not limited to the enzymes from algae (C. reinhardtii), bird (chicken, duck etc.), fruit fly (e.g. Drosophila melanogaster), fish (e.g. zebra fish), mammal (mouse, rat, rabbit, dog, horse, goat, sheep and human), and fungus (e.g. Fusarium fujikuroi). The DNA and proteins sequences of these enzymes are available from the NCBI GeneBank which are incorporated herein by reference.

Three C. reinhardtii genes identified from the C. reinhardtii genome sequence by homology to previously characterized β-carotene:oxygen 15,15′-monooxygenase genes will be expressed and targeted to the thylakoid membrane. Reduction of retinal to retinol may occur due to broad-specificity aldehyde NAD(P)H reductases that are basally expressed in the chloroplast.

NinaB from Drosophila encodes a β-carotene oxygenase that produces retinal in adult fly brains, and is 34% identical to the chicken version that can be used in the production of retinal, retinol and vitamin A. U.S. 2003/0166595 A1.

The carotenoid oxygenase CarX from Fusarium fujikuroi has been shown to possesses β-carotene cleaving activity to produce retinal. Prado-Cabrero et al., Eukaryote Cell. (2007) 6(4): 650-657.

An orthologous carX protein in Ustilago maydis, called CCO1 is disclosed in Estrada et al., Fungal Genet Biol. (2009) 46(10):803-13.

The chicken carotene 15,15′-monooxygenase is disclosed in U.S. Pat. No. 6,897,051.

The transit peptides used to target β-carotene:oxygen 15,15′-monooxygenase and a retinol:NAD+ oxidoreductases to the lumen of the chloroplast and to the thylakoid membranes include but are not limited to ZEP1 from C. reinhardtii (XM_{—}001701649.1), CHYB from C. reinhardtii (XM_{—}001698646.1), PETF from C. reinhardtii (XM_{—}001692756.1), HLP from C. reinhardtii (NW_{—}001843472.1.

A list of genes and transit peptides used in the production of retinol according to the present invention is provided in Table 2 and Table 3.

TABLE 2

| Gene or Protein | Organism | Accession Number |
|---|---|---|
| BCMO1 | Homo sapiens | NP_059125.2 |
| Blh | Uncultured marine | AAY68319.1 |
| | bacterium 66A03 | |
| CarX | Gibberella | CAH70723.1 |
| | fujikuroi | |
| NinaB | Drosophila | NP_650307.2 |
| | melanogaster | |
| CHLREDRAFT_144745 | Chlamydomonas | XM_001691049.1 |
| | reinhardtii | |
| CHLREDRAFT_141185 | Chlamydomonas | XM_001701568.1 |
| | reinhardtii | |
| CHLREDRAFT_141186 | Chlamydomonas | XM_001701569.1 |
| | reinhardtii | |

TABLE 3

| Transit peptide | Organism | Accession Number |
|---|---|---|
| ZEP1 | Chlamydomonas reinhardtii | XM_001701649.1 |
| CHYB | Chlamydomonas reinhardtii | XM_001698646.1 |
| PETF | Chlamydomonas reinhardtii | XM_001692756.1 |
| HLP | Chlamydomonas reinhardtii | NW_001843472.1 |

### B. Omega-3 FA (DHA/EPA)

In another aspect, the present invention provides compositions and methods for the production of sustainable and low-cost DHA/EPA in triglyceride form in cell, such as in an alga. In some embodiments, the method comprises introducing a non-native biosynthetic pathway for generating FAs, such as DHA and EPA, in eukaryotic algae.

Docosahexaenoic acid (DHA, 22:6 Δ4, 7, 10, 13, 16, 19) and eicosapentaenoic acid (EPA, 22:5Δ7, 10, 13, 16, 19) are 22 carbon FAs with and 6 and 5 unsaturated carbon-carbon bonds, respectively. These fatty acids are important for human health and need to be present in the human diet. They support brain, eye and heart health throughout all stages of life. The strongest evidence for health benefits of ω-3 FAs relates to cardiovascular health and cognitive performance. DHA and EPA can be obtained from animal sources, like fish oil, and from vegetarian sources, like algae. While fish sources are less expensive sources of lower quality ω-3 FAs, prices continue to rise and fish stocks continue to be depleted. Vegetarian sources of DHA are significantly better because they are sustainable, do not add unpleasant fish odor, and are free of toxic impurities such as PCBs and mercury. The National Institute of Health and the American Heart Association have recommended daily targets for minimal DHA intakes, but there is a nutrition gap between actual and targeted intakes for different age groups ranging from 70% to 80% as the cost of vegetarian DHA/EPA is too expensive (about $5,600 per year for recommended daily intake for a family of four). This high cost creates a need for affordable sources of high-quality, contaminant-free ω-3 FAs for people in lower socioeconomic groups.

C. reinhardtii lacks FAs longer than 18 carbons. However, α-linolenic acid (ALA), an 18 carbon ω-3 FA with three unsaturations, makes up 16% of all FAs in this organism and can serve as an abundant precursor for DHA/EPA. Tatsuzawa et al., Journal of Phycology (1996) 32:598-601.

Throughout biology, fatty acids longer than 18 carbons, such as DHA, are synthesized by the addition of 2-carbon units to pre-existing fatty acids by a complex of enzymes called very long chain fatty acid (VLCFA) synthase, which is similar to the complex that synthetizes fatty acids up to 16 and 18 carbons, fatty acid synthase (FAS). Both FAS and VLCFA synthase catalyze a similar series of 4 reactions: condensation, reduction, dehydration and reduction but, while FA synthase stops the sequence when the FA chain is 16- or 18-carbons long, VLCFA synthase can elongate existing FAs to reach lengths of up to 36 carbons. Each enzymatic activity in VLCFA synthase is present in a different polypeptide. The enzymes that catalyze the 2^{nd}, 3^{rd} and 4^{th} reactions are similar to those of FAS, however the first enzyme, i.e. the enzyme catalyzing the carbon-carbon bond-forming Claisen-condensation, is not homologous to the corresponding module in FAS. The length specificity of VLCFA synthase is provided by the enzyme that catalyzes first step in the elongation cycle, which is typically referred to as the condensing enzyme, or more commonly, the “elongase.” There are many different elongases in plants, animals, and other organisms, each one able to elongate up to a certain length.

This invention makes use of two different elongases to extend the chain-length from 18 carbons (typical of the FA's made by C. reinhardtii) to 22 carbons.

The synthesis of fatty acids with insaturations it catalyzed by enzymes called fatty acid desaturates, commonly simply called “desaturases”. Desaturases remove two hydrogen atoms from adjacent carbons at a specific position in the fatty acid substrate, creating a carbon/carbon double bond at that position. Each desaturase acts on fatty acids of a specific length and introduces a double-bond only at a specific position. The rich variety of poly-unsaturated fatty acids (PUFAs) in plans and animals is generated by the sequential action of different desaturases during the synthesis of the fatty acids. Desaturases alternate with elongases in the biosynthetic pathway of PUFAs.

This invention makes use of three different reductases to introduce 3 different insaturations in a biosynthetic pathways that leads from a fatty acids with 3 insaturations, ALA, to DHA, which has 6 insaturations.

In some embodiments, the cell is transformed with an elongase gene and a desaturase gene. In some embodiments, the cell is transformed with a Δ6-desaturase gene, a Δ6-elongase gene, a Δ5-desaturase gene, a Δ5-elongase gene, and a Δ4-desaturase gene.

In some embodiments, the production of DHA is by the expression of one or more genes selected from the group consisting of: Fat-3, Elo-2, Fat-4, elo (from Pavlova Sp), and IgD4.

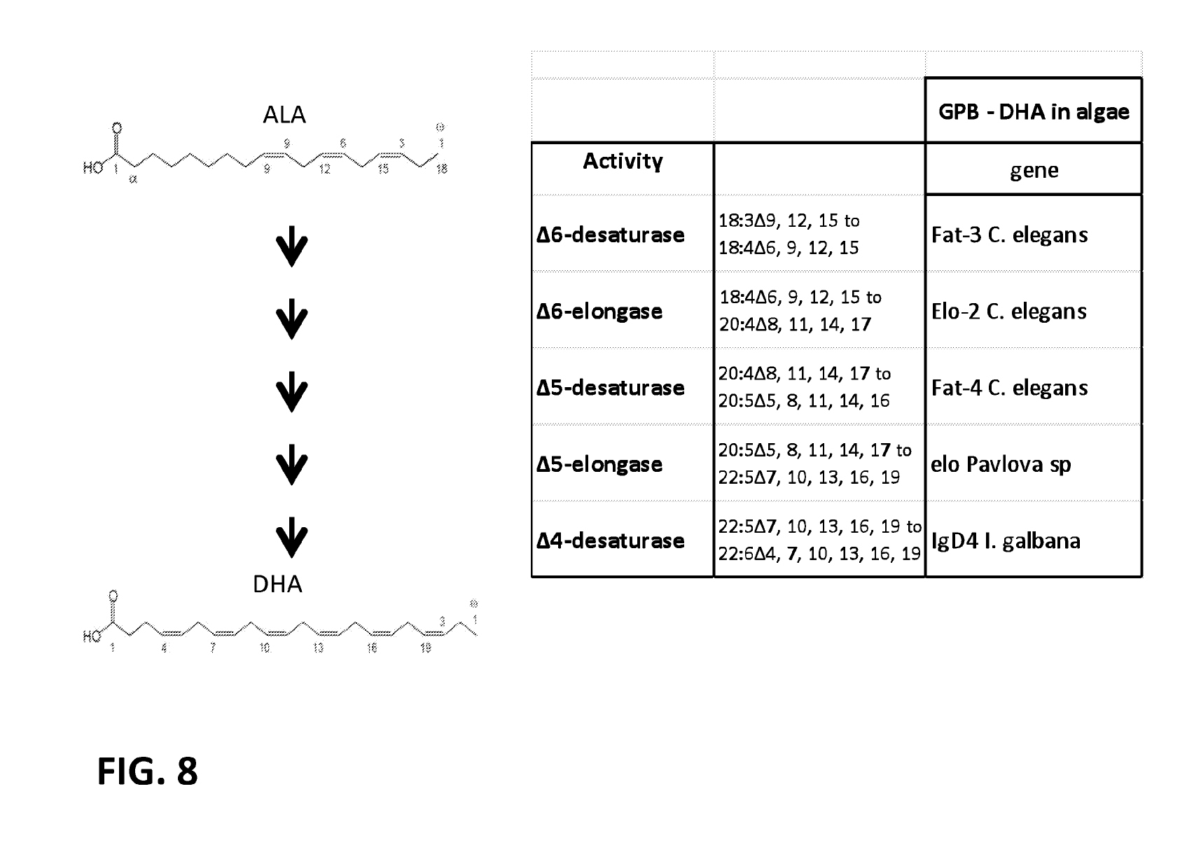

The DHA synthesis pathway includes five steps (FIG. 8):

Step 1. Δ-6 Desaturation: Convert ALA to Stearidonic Acid (SDA)

Fat-3 is the Δ6-desaturase gene from C. elegans and is disclosed in U.S. Pat. No. 6,825,017.

Δ6-desaturase from Micromonas pusilla is discloses in Petrie et al., Plant Methods. (2010)6:8.

Step 2. Δ-6 Elongation: Conversion of SDA to Eicosatetraenoic Acid (ETA)

Elo-2 gene from C. elegans is disclosed in Kniazeva et al., Genetics, 2003. 163(1):159-69, and Watts & Browse, Proc Natl Acad Sci USA, 2002. 99(9):5854-9.

Δ6-elongase from Pyramimonas cordata is disclosed in Petrie et al., Plant Methods. (2010) 6:8 and US20050273885A1.

Step 3. Δ-5 Desaturation: Conversion of ETA to EPA

Fat-4 gene from C. elegans 1 is disclosed in Watts and Browse, Arch Biochem Biophys. (1999)362(1):175-82, Michaelson et al., FEBS Lett. (1998)439(3):215-8, Beaudoin et al. Proc Natl Acad Sci USA. (2000) 97(12):6421-6, and U.S. Pat. No. 6,825,017.

Δ5-desaturase from P. Salina is disclosed in Zhou et al., Phytochemistry. (2007) 68(6):785-96, and Plant Methods. (2010) 6:8.

Step 4. Δ-5 Elongation: Conversion of ETA to Docosapentaenoic Acid (DPA)

elo gene from Pavlova sp. is disclosed in Pereira et al., Biochem J. (2004) 384(Pt 2):357-66. Expression of the elo gene in yeast enabled the elongation of C20 PUFA to C22.

Δ5-elongase from P. cordata is disclosed in Petrie et al., Mar Biotechnol (NY). 2009 Oct. 10, and Petrie et al., Plant Methods. (2010) 6:8.

Step 5. Δ-4 Desaturation: Conversion of DPA to DHA

IgD4 from Isochrysis galbana is disclosed in Pereira et al., Biochem J. (2004) 384(Pt 2):357-6. Expression of the IgD4 and the elo gene from Pavlova sp. in yeast enabled the synthesis of DHA.

Δ4-desaturase from P. Salina is disclosed in Petrie et al., Plant Methods. (2010) 6:8 and Zhou et al., Phytochemistry. (2007)68(6):785-96.

In some embodiments, the method comprises engineering cells (e.g. algae) to express a desaturase (e.g. FAT3) and an elongase (e.g. ELO2) to convert naturally occurring C18:3 (18:3Δ9, 12, 15) to C20:4 (C20:4Δ8,11,14,17), a direct precursor of DHA, in the algae cell. The cells synthesize detectable levels of C20:4.

In some embodiments, the genetically engineered cells produce a non-native FA intermediate in DHA/EPA biosynthesis, C20:4Δ8,11,14,17, and produce 10%, 20%, 30%, 40%, 50%, 60%, 70%, 80%, 90%, or 100% more total TG than unengineered cells.

In some embodiments, the genetically engineered cells further express additional genes (e.g. Fat-4, elo, and IgD4) to convert the FA intermediate C20:4Δ8,11,14,17 into EPA and DHA.

In some embodiments, the cells are further engineered to increase the TG production, such as to express increased level of DGAT2, thereby maximize the biosynthesis of DHA and EPA in triglyceride forms.

### V. Method of Production

### Large-Scale Algae Biofuel Production

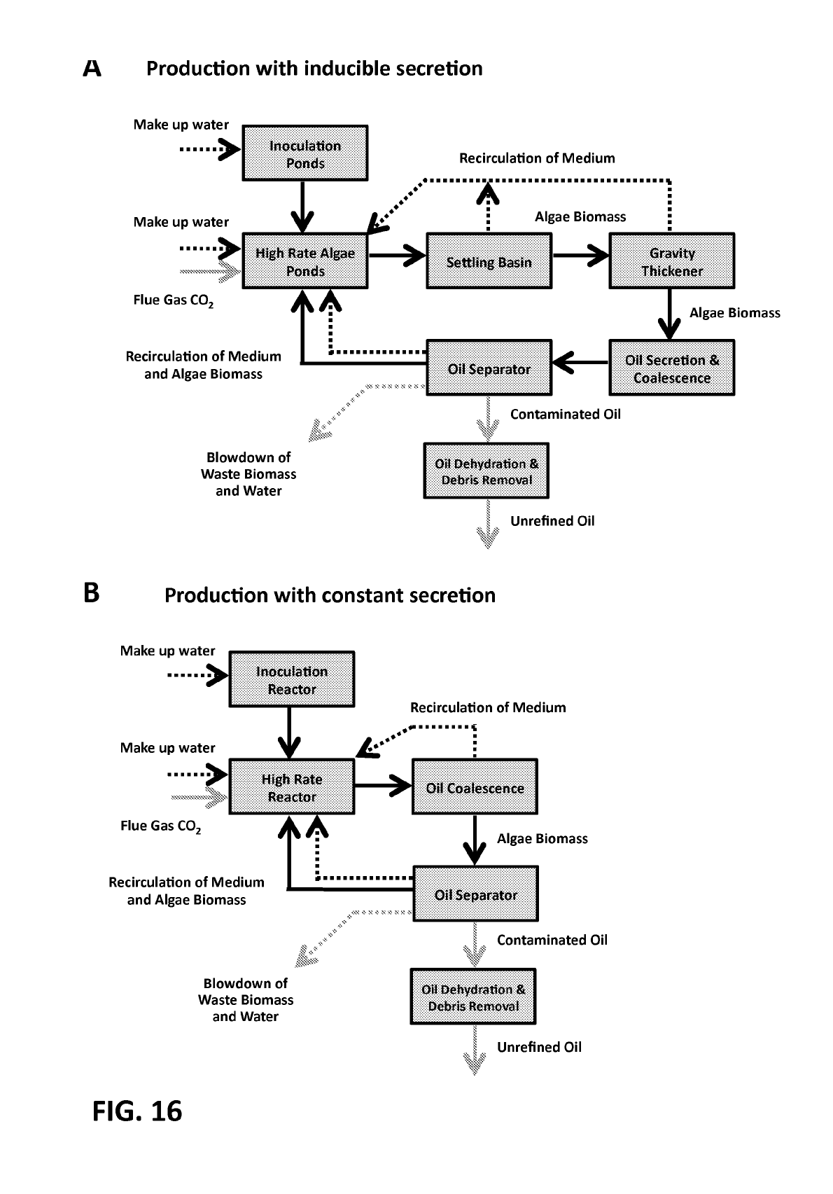

In one aspect, the present invention describes production facilities to be used in large-scale algae biofuel production. The production facilities described in the invention are designed to exploit the genetically modified algae strains described in the preceding sections. These genetic modifications improve the biofuel production process. The productions facilities and the systems of production that utilizes them are described below and shown schematically in FIG. 16.

The algae strains used in the production systems described here is one that has been modified to i) have an increased flux of CO₂ to triglycerides and ii) secrete membrane-bound triglyceride packages, herein referred to as “TAG globules”, to the extracellular environment.

Two main types of production systems are described, both constitute alternative embodiments. One type of production system will use open raceway ponds, also known as high rate ponds (HRPs), each with an area of several hectares. A second type will use enclosed growth vessels known as photobioreactors (PBRs). In the PBRs the algae will be cultivated in, for example, transparent plastic bags or plastic tubes with pumps to promoter circulation.

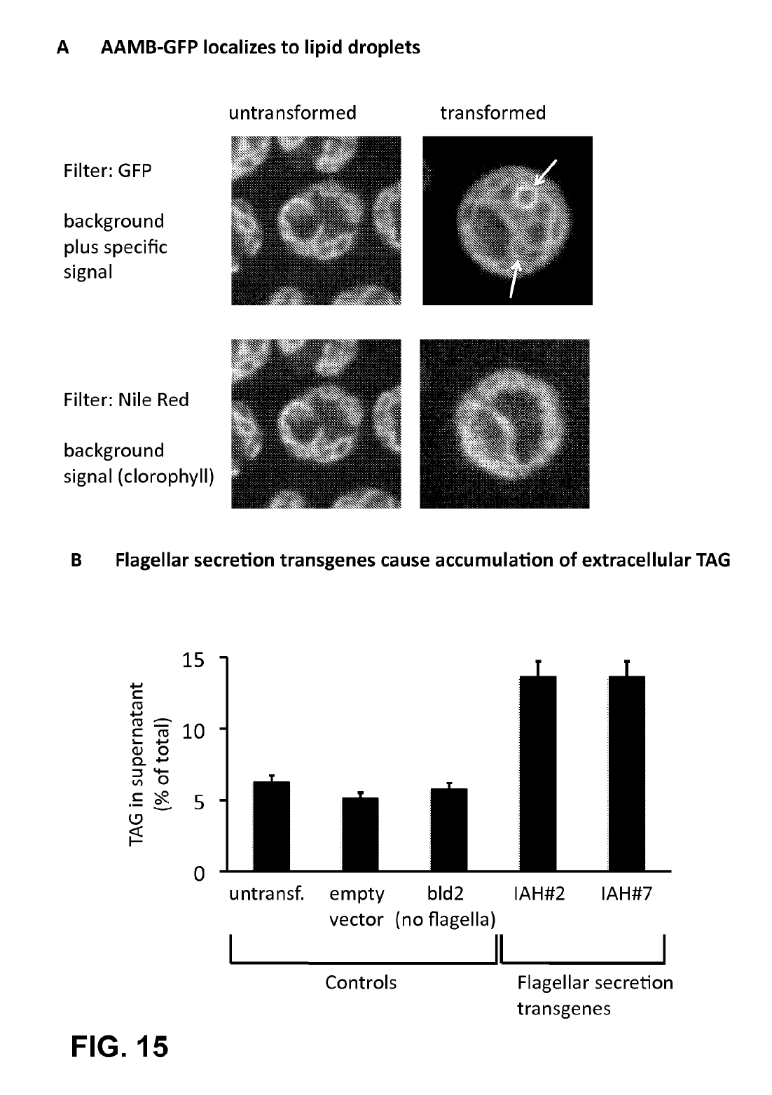

In one aspect of the design of the production system that exploits these advantages the secreted triglycerides are obtained from the algae without damaging the algae, which can continue to produce triglycerides. The two schematics shown in FIG. 15 represent alternative embodiments of this approach as applied to HRPs and to PBRs. In both the HRPs and PBRs systems the algae recovered and the undamaged algal cells are returned to the production ponds for further production cycles. As a consequence of the recycling of the cells, the production systems benefit from a dramatic improvement in energy efficiency, as there is no energy needed to produce new cells. In such non-destructive production systems the algae act as a catalyst that converts CO₂ and sunlight into triacylglycerides efficiently, without being consumed in the process. By eliminating the energy investment in cell growth the efficiency of the conversion of captured solar energy into triglcyerides is increased from 30% to 97%.

In another aspect that exploits the improved characteristics of the genetically modified algae the triglycerides are purified from the extracellular medium by simple and inexpensive concentration and extraction steps. This is in contrast with current procedures that require costly and time consuming steps to separate the algae cells from the media, dehydrate them and break them open to extract the triglyceride stored inside the cells. The elimination of the cell harvesting, drying and extraction costly steps results in about 30% reduction in capital costs and 22% reduction in operating costs.

The HRPs production system will cultivate microalgae in many identical ponds, each with an area of several hectares, as limited by hydraulic considerations and the need for redundant cultures. These individual HRPs will be clustered in facilities with total pond areas upwards of several hundred hectares. The individual HRPs will operate independently and will be connected to a centralized biomass processing area through a piping network that distributes make-up water, inoculum, and recycled flows. The HRPs will be shallow (˜30 cm deep) channelized raceways each with a single paddle wheel mixing station to provide a channel flow rate of 20-30 cm/s. Each pond will incorporate one or more carbonation stations where CO₂— rich gas (e.g., flue gas) can be introduced to promote algae growth. The HRPs will be lined with native clay to decrease seepage of the growth media.

The PBR production system will cultivate microalgae in translucent tubes, hoses or plastic bags each with a volume ranging from 100 to 1000 liters. In some PBRs configurations, the plastic bags are submerged as a series of panels that maximize light penetration and efficient mixing of CO₂. The close systems are integrated with control systems that record key parameters, such as pH and CO₂ levels, and automatically regulate mass flux. This additional level of control provides higher efficiencies such as higher growth rates and cell mass yields. The PBRs will be connected to a centralized biomass processing area.

In a preferred embodiment, the make-up water and nutrients for the production system will be municipal wastewater, which will minimize costs. In alternative embodiments, the wastewater growth medium could be replaced with media comprised of saline, brackish or, where available, fresh water. In these cases, the medium will be supplied with agricultural fertilizers, which will increase costs.

Evaporation of the water in the media and the associated increase in salinity will be compensated by addition of fresh water and blow down (i.e. elimination or discharge) of some growth media from the oil separator units. To prevent excessive build-up of non-viable cell material, some of the biomass will be removed if a sufficient amount is not removed in the blow-down. Blow-down of biomass is described further below. Evaporation of the water is not an issue in the closed-PBR system.

In order to promote the initial dominance of the inoculated, genetically modified algal strains over “weed” species of algae (i.e. local, naturally occurring algae), the production cultures will be inoculated with a large amount of innoculated algal cells. The inoculation culture will be grown up initially in sterilized photobioreactors and then in a succession of larger photobioreactors. For the HRPs system, the photobioreactors will be followed by small covered HRPs and then final uncovered inoculation ponds, which will be similar in design to the large HRP production ponds, except that they will be lined with plastic to allow periodic cleaning to decrease contamination.

In one embodiment the secretion of triglycerides will be induced at a specific point in the production process. This is especially well suited for the HRPs system, for this reason it is shown as the embodiment of choice in the HRP flow diagram in FIG. 16. In the embodiment that adopts the induction of secretion approach and following TAG biosynthesis during the day, the medium will be piped to flocculating-settling basins and gravity thickeners, where cells will be concentrated in a smaller volume, and later to secretion and coalescence reactors, where the TAG globule release from the algal cells will be induced, and TAG globule coalescence will occur. Induction of TAG released in such a specific reactor will minimize bacterial degradation of TAGs that would occur in a process with continuous TAG release in the growth pond. In this embodiment, as applied to the HRPs system, optimal cultivation of specific algal strains will require a daily cycle of operation. Algae will be harvested daily from the HRPs at the end of the day (i.e., daily semi-continuous mode) to avoid nighttime respiration losses. Depending on season and climate, a typical HRP hydraulic retention time will be 2-5 days (20-50% dilution per day).

The released TAG globules are expected to be coated or membrane-bound and 1-5 μm in diameter (similar to milk TAG). During milk separation, oil globules have been seen to separate under simple gravity without coalescence (Ma and Barbano 2000) and thus efficient separation of fat as a floating layer will be possible in the production systems described here. However, in a preferred embodiment technologies that promote coalescence will be used to accelerate oil separation. In different instantiations, coalescence will be promoted by either or both physical and chemical means. A physical method will be similar to the one used in the petroleum industry in which an oil-water mixture is passed through fibrous or granular beds (e.g., Gu and Li 2005). A chemical method that will be used will maintain high concentrations of calcium ions or other agents (Valivullah et al. 1988).

In the embodiment of the HRPs system that includes a step to induces the release of TAG globules at a specific step, the two processes of triggering of TAG release followed by TAG coalescence will occur in a single type of reactor—plastic-lined, packed-bed ponds. The gravity thickener subnatant will be subjected to induction of secretion and then passed up through the packed-bed coalescing medium of these ponds.

The water-algae-TAG mixture in the effluent of the induction/coalescence units will be piped into oil separation units. In a preferred embodiment, simple gravity oil-water separators similar to those used in the petroleum and wastewater treatment industries will be used. At the surface of the separator units, mechanical skimmers and collection sumps will collect the TAG oil, with the water-biomass fraction leaving the tanks near the floor. This water-biomass solution will be mainly recycled to the ponds, to allow for the next cycle of TAG production.

A portion of the oil separator underflow, in a preferred instantiation 2% per day, will be continuously disposed of as blowdown to reduce the load of dead and refractory cells in the system. Approximately once a month, the entire biomass will be replaced with a fresh inoculum. These biomass losses represent and approximate loss of 5% per day of captured solar energy.

Blowdown of liquid or liquid-biomass mixtures will also serve to control the salinity of the growth medium. In one embodiment in which seawater is used as the medium, at least 5% per day may need to be blown-down during summer. In the embodiments that include discharge to water bodies, the algal biomass in the blowdown will be removed by slow sand filters. In other embodiments as applied to the HRPs system blowdown disposal will be done in evaporation ponds.

The production systems will include methods to remove solids and water from the oil skimmed from the oil separator. These undesired solids include all instances of incidental solid matter, including unsettled algae and debris. Solids will be removed by sedimentation, flotation, or filtration. For simplicity of description sedimentation will be assumed herein. The water removal will require more than plain gravity separation, but unlike the oil separator described above, the oil content at this stage will be greater than the water content. To remove the water, two main technologies used in the oil industry for dehydrating crude oil will be used: heater treaters and electrostatic separators. In one embodiment the production process will use devices integrating the two methods. Heater treaters are tanks that are heated to promote further coalescence and to enhance the density difference of oil and water. Operating temperatures range from 32-120° C. depending the oil-growth medium density difference and the type of emulsion. In one embodiment waste heat from electrical generators will be used, especially at the lower temperatures. Such generators will also be a source of CO₂ for the algae production. Electrostatic units use AC electrodes to repeatedly distend and relax water droplets, which promotes coalescence. Heater treaters greatly accelerate separation: Whereas plain gravity separation in “wash tanks” usually requires 8-24 hours of retention time, heater treaters and electrostatic units require 0.25-4 hours. In addition, demulsifying agents such as proprietary surfactant blends will be added to promote coalescence and decrease the needed retention time, heat, and/or power required. Heater treaters and electrostatic units will also employ mechanical mixing, baffles, lamella, and physical media to enhance coalescence. In one embodiment, an additional step of centrifugation will be used as a polishing step in dehydration, or also as an alternative to accelerate separation. The choice of methods of oil dehydrating will depend mainly on the relative oil-water content, water salinity, and emulsion type. For simplicity, the heater treater technology is assumed in this description, with provision of free waste heat. After dehydration and removal of solids, a crude vegetable oil will have been produced for shipment.

The production systems described do not need to include additional steps to handle biological waste. In contrast with standard algae production systems, the waste biomass flow in this production system is too small to warrant anaerobic digestion. It is a tremendous advantage of this production system to have less waste to handle than standard algae production systems.

Quantitative estimation of productivity: The production systems combining the described technologies for oil secretion, for increased oil synthesis without tradeoffs in growth rates, and for increased algae light utilization can reduce costs to around $50/bbl with HRPs and to around $60/bbl with PBRs. We have evaluated the cost of algae oil production at commercial scales in this production systems using a techno-economic model developed at GPB. Table 4 summarizes the cost per barrel at different production scales for different parameters of the bioengineered solutions described in this patent using HRPs or PBRs as the production platform.

TABLE 4

| | | | | Photosynthetic | | Price |
|---|---|---|---|---|---|---|
| | | | % cell oil | efficiency (% energy | Yield: | (Cost/ |
| | | Secretion | content | converted to mass) | gal/acre/year | barrel) |
| BR | Lab | 15% | 15% | 2.6% | 1667 | $504 |
| | Pilot | 25% | 25% | 2.6% | 4629 | $181 |
| | Demonstration | 30% | 40% | 5.2% | 8888 | $71 |
| | | 50% | 25% | | 9259 | $68 |
| | | 90% | 15% | | 10000 | $63 |
| | Commercial | 30% | 40% | 5.2% | 8888 | $71 |
| | | 50% | 25% | | 9259 | $68 |
| | | 90% | 15% | | 10000 | $63 |
| HPR | Lab | 10% | 15% | 2.6% | 549 | $924 |
| | Pilot | 20% | 25% | 2.6% | 1829 | $277 |
| | Demonstration | 90% | 29% | 5.2% | 9550 | $53 |
| | | 35% | 55% | | 7043 | $72 |
| | | 50% | 40% | | 7318 | $69 |
| | | 90% | 25% | | 8233 | $62 |
| | Commercial | 90% | 29% | 5.2% | 9550 | $53 |
| | | 35% | 55% | | 7043 | $72 |
| | | 50% | 40% | | 7318 | $69 |
| | | 90% | 25% | | 8233 | $62 |

The techno-economic model is primarily an energy balance on the production system (pond or PBR). In simple terms, the energy in (via captured solar energy) must equal the energy out (via secretion of TAG, daily loss of biomass, and energy lost as heat). Additionally, the model incorporates mass balances and parameter restrictions to ensure the system is physically realistic and biologically feasible.

The assumptions used in the techno-economic model were as follows:
Total average solar insulation: 4500 kcal per m² per day
Secreted triacylglyceride (TAG) has an energy content of 9.1 kcal/g, while non-TAG algae biomass has an energy content of 4.8 kcal/g
Working (saturated) algae cell density (dry weight): PBRs=3 g/L, HRPs=0.3 g/L
Liquid culture volume per square meter: PBRs=60 L, HRPs=300 L
Operating cost per acre per year: PBRs=$15,000, HRPs=$12,080 (GPB), $12,880 (Base case)
Average daily loss of algal cell biomass (due to periodic cleaning, waste, blowdown): PBRs=1%, HRPs=5%
The modeled system operates in a semi-continuous mode, in which the synthesized TAG is harvested daily (TAG could either be continuously secreted or TAG secretion could be induced prior to harvesting).

The modeled system is stable, in the sense that the pond or PBR always reaches the same net energy content at the end of every day (prior to harvesting). Therefore, the algae always reach the same TAG content at the end of every day (i.e., they do not accumulate or lose TAG over longer time periods). Gu, Y. and J. Li (2005) “Coalescence of oil-in-water emulsions in fibrous and granular beds,” Separation and Purification Technology, 42, pp. 1-13; Ma Y. and D. M. Barbano (2000). Journal of Dairy Science; Valivullah, H. M., D. R. Bevan, A. Peatt, and T. W. Keenan (1988). “Milk lipid globules: Control of their size distribution,” Proc. Nat. Acad. Sci., Applied Biological Sciences, Vol. 85, pp. 8775-8779.

Converting Lipids into Biofuel

In one aspect, the present invention provides a biofuel, a biodiesel, or an energy feedstock comprising lipids derived from algae.

Examples of systems and methods for processing lipids such as algal oil into biofuel, can be found in the following patent publications, the entire contents of each of which are incorporated by reference herein: U.S. Patent Publication No. 2007/0010682, entitled “Process for the Manufacture of Diesel Range Hydrocarbons;” U.S. Patent Publication No. 2007/0131579, entitled “Process for Producing a Saturated Hydrocarbon Component;” U.S. Patent Publication No. 2007/0135316, entitled “Process for Producing a Saturated Hydrocarbon Component;” U.S. Patent Publication No. 2007/0135663, entitled “Base Oil;” U.S. Patent Publication No. 2007/0135666, entitled “Process for Producing a Branched Hydrocarbon Component;” U.S. Patent Publication No. 2007/0135669, entitled “Process for Producing a Hydrocarbon Component;” and U.S. Patent Publication No. 2007/0299291.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### A. Materials and Methods

General methods: PCR and general molecular biology methods were performed as described in Ausubel, F. M. Current Protocols in Molecular Biology. (Greene Pub. Associates: 1988), except that for PCR reactions 1M betaine was typically included.

Strains: all strains/cell lines were constructed from Elo47, UVM4, or UVM11², all of which are derived from cw15 cells. Neupert et al., J., Plant J 57:1140-1150 (2009).

### Plasmid Construction

pGPB1012 (P_{Psad}-N/E-GFP). pGPB1012 were constructed from pJR38². Neupert et al., J., Plant J 57:1140-1150 (2009). We excised GFP from pJR38 by removing the small NdeI/EcoRI fragment. We PCR amplified GFP with primers that added in-frame NdeI+EcoRI sites at the 5′ end and a TAA stop codon and MfeI site at the 3′ end. We then digested the PCR product with NdeI and MfeI, and ligated into the large NdeI/EcoRI fragment of pJR38.

pGPB1013 (P_{PsaD}-N/E-HA). We constructed pGPB1013 as described for pGPB1012, except that instead of amplifying GFP, we amplified the 3-tandem copy HA tag from p3xHA (Chlamydomonas Resource Center).

pGPB1014 (P_{Rbcs2}-N/E-GFP). We constructed pGPB1014 as described for pGPB1012, except that we cloned the NdeI/MfeI digested GFP PCR product into the large NdeI/EcoRI fragment of pJR40. Neupert et al., J., Plant J 57:1140-1150 (2009).

pGPB1015 (P_{Rbcs2}-N/E-HA). We constructed pGPB1014 as described for pGPB1012, except that we cloned the NdeI/MfeI digested GFP PCR product into the large NdeI/EcoRI fragment of pJR40. Neupert et al., J., Plant J 57:1140-1150 (2009).

pGPB1001 (synthetic human Dgat2 in pUC57). We synthesized Chlamydomonas-codon optimized human Dgat2 based on accession number NM_{—}032564 (Genscript).

pGPB1017 (P_{PsaD}-hDgat2-HA). We constructed pGPB1017 by amplifying the human Dgat2 (hDgat2) open reading frame with primers adding an in-frame NdeI site at the 5′ end and an EcoRI site at the 3′ end, digesting this PCR product with NdeI and EcoRI, and cloning the digested product into the NdeI/EcoRI site of pGPB1013.

pGPB1026 (P_{PsaD}-crDgat2B-HA). We constructed pGPB1026 by amplifying a possible Chlamydomonas Dgat2 homologue (PID 190539) from genomic DNA. We amplified this 3.6 kb gene by PCR using primers that added an in-frame NdeI site at the 5′ end and an EcoRI site at the 3′ end. We digested this PCR product with NdeI and EcoRI, and cloned the digested product into the NdeI/EcoRI site of pGPB1013.

pGPB1032 (P_{PsaD}-BTN-GFP) and pGPB1033 (P_{PsaD}-BTN-HA). We synthesized Chlamydomonas-codon optimized human butryophillin 1A1 (BTN) (NP_{—}001723.2) (Genscript). We PCR amplified the BTN open reading frame using primers that added an in-frame NdeI site at the 5′ end and an EcoRI site at the 3′ end. We digested this PCR product with NdeI and EcoRI, and cloned the digested product into the NdeI/EcoRI site of pGPB1012 and pGPB1013 to make pGPB1032 and pGPB1033, respectively.

pGPB1034 (P_{PsaD}-ADPH-GFP). We synthesized Chlamydomonas-codon optimized human adipophillin (ADPH) (Accession No. CAA65989) (Genscript). We PCR amplified the ADPH open reading frame using primers that added an in-frame NdeI site at the 5′ end and an EcoRI site at the 3′ end. We digested this PCR product with NdeI and EcoRI, and cloned the digested product into the NdeI/EcoRI site of pGPB1012 to make pGPB1034.

pGPB1038 (P_{PsaD}-MLDP-GFP-STX) and pGPB1056 (P_{Rbcs2}-MLDP-GFP-STX) We constructed MLDP-GFP-STX by rounds of PCR and PCR fusion. We first PCR amplified the open reading frame of MLDP (XP_{—}001697668) from total cDNA using primers that added an in-frame NdeI site at the 5′ end and an NruI site and a sequence overlapping with the 5′ end of GFP (as found in pJR38) at the 3′ end. We also PCR amplified GFP from pJR38 using primers that added at the 5′ end a sequence that anneals to the 3′ end of MLDP and an NruI site, and at the 3′ end an NruI site and a tail that anneals to the 5′ end of STX. Finally, we PCR amplified the open reading frame of Chlamydomonas syntaxin 1 (STX) (XP_{—}001693638) from total cDNA using primers that added a sequence that anneals to the 3′ end of GFP and an NruI site at the 5′ end, and an TAA stop codon and an EcoRI site at the 3′ end. We then fused by PCR the three fragments. We first PCR fused MLDP to GFP to form MLDP-GFP, and then PCR fused MLDP-GFP to STX to for MLDP-GFP-STX. We digested this fused PCR product with NdeI and EcoRI, and cloned the digested product into the NdeI/EcoRI site of pJR38 to make pGPB1038. We also cloned the digested product into the NdeI/EcoRI site of pJR40 to make pGPB1056.

pGPB1042 (P_{PsaD}-GFP-STX) and pGPB1058 (P_{Rbcs2}-GFP-STX). We PCR fused the GFP and STX PCR fragments as described above in the construction details of pGPB1038. We then PCR amplified this GFP-STX product with primers that added an NdeI site at the 5′ end and a stop codon followed by an EcoRI site at the 3′ end. We digested this fused PCR product with NdeI and EcoRI, and cloned the digested product into the NdeI/EcoRI site of pJR38 to make pGPB1042. We also cloned the digested product into the NdeI/EcoRI site of pJR40 to make pGPB1058.

pGPB1050 (P_{PsaD}-MLDP-GFP) and pGPB1052 (P_{Rbcs2}-MLDP-GFP). We PCR amplified the open reading frame of MLDP (XP_{—}001697668) from pGPB1038 using primers that added an in-frame NdeI site at the 5′ end and an EcoRI site at the 3′ end. We digested this PCR product with NdeI and EcoRI, and cloned the digested product into the NdeI/EcoRI site of pGPB1012 to make pGPB1050. We also cloned the digested product into the NdeI/EcoRI site of pGPB1014 to make pGPB1052.

pGPB1037 (P_{PsaD}-IFT20-AAM-B-HA): We PCR amplified the IFT20 gene (XM_{—}001701914) and PCR fused to the 3′ end of this product a DNA fragment encoding the first 40 amino acids of human AAM-B protein (CAD60207). This PCR fusion product contained an in-frame NdeI site at the 5′ end and an EcoRI site at the 3′ end. We digested this PCR product with NdeI and EcoRI, and cloned the digested product into the NdeI/EcoRI site of pGPB1013 to make pGPB1037.

pGPB1092 (P_{PsaD}-IFT20-MLDP-HA: We PCR amplified the IFT20 gene (XM_{—}001701914) and PCR fused it to the 5′ end of MLDP (from pGPB1050). This PCR fusion product contained an in-frame NdeI site at the 5′ end and an EcoRI site at the 3′ end. We digested this PCR product with NdeI and EcoRI, and cloned the digested product into the NdeI/EcoRI site of pGPB1013 to make pGPB1092.

pGPB1029 (P_{PsaD}-SpeI-reverse P_{PsaD}): We digested pJR38 (Neupert et al., 2008) with SpeI, treated with Klenow fragment to fill in ends, and religated. We then digested this plasmid with NdeI and EcoRI, and cloned into these restriction sites a PCR product of the reverse complement of the PsaD promoter with a 5′ NdeI-SpeI containing tail and a 3′ EcoRI-containing tail after digestion with NdeI and EcoRI.

pGPB1062 (P_{PsaD}-Sta6-reverse P_{PsaD}): We prepared cDNA from Chlamydomonas reinhardtii and PCR amplified from the cDNA the Sta6 coding sequence, with a 5′ SpeI containing tail and a 3′ SpeI containing tail. We then digested this PCR product with SpeI and then ligated into pGPB1029 digested with SpeI.

pGPB1043 (P_{PsaD}-GFP with HygB resistance casette): We digested pJR38 with HindIII and KpnI. We then PCR amplified the hygromycin B resistance casette from pKS-aph7″-loxP (from http://www.chamy.org) using primers that introduced a 5′ HindIII site and a 3′ KpnI site. We then digested the PCR fragment with HindIII and KpnI and ligated it into the HindIII/KpnI digested pJR38.

pGPB1064 (P_{PsaD}-Sta6-reverse P_{PsaD} w/HygB resistance casette): We subcloned the ClaI/KpnI fragment from pGPB1043 into the ClaI/KpnI sites of pGPB1062.

Total RNA purification and cDNA pool construction: We purified total RNA from CC125 cells (Chlamydomonas Resource Center) by extracting nucleic acids from cells using Trizol, as described by manufacterer's directions, treating with DNase, and purification using a Nucleospin RNA Plant kit (Clontech). We then made cDNA using a SMARTScribe kit (Clontech).

Algae culturing and transformation: We grew algae both on solid and in liquid media. For solid media, we used TAP³+2% Bacto-agar, supplemented in certain situations as noted in appropriate locations of the methods. We maintained cell lines containing selective markers on selective solid media (TAP+2% Bacto-agar+10 μg/ml paromomycin and/or 10 μg/ml hygromycin B) unless otherwise noted. Plates were kept in clear plastic boxes at room temperature under standard 4 foot, two-bulb fluorescent lights equipped with standard cool white bulbs. For liquid cultures, we used either plain TAP or TAP supplemented with 3.3 μg/ml paromomycin or 100 μg/ml arginine. We grew liquid cultures, typically between 1 and 10 ml, in 16 mm×200 mm glass tubes in a rotator drum under fluorescent light.

To integrate gene expression cassettes into the nuclear genome, we transformed 0.2-1 μm linear plasmid DNA into cells from 10-20 ml of culture at late-log growth or early saturation usng the previously described glass bead vortexing method⁴. We recovered each transformation reaction in 10 ml TAP for 24-48 hours, then plated on solid media containing the appropriate selective drug as described above.

All chemicals were purchased from Sigma-Aldrich unless otherwise noted.

### Quantification of Intracellular Oil by Microscopy

We measured intracellular oils using fluorescence microscopy by quantifying Nile Red fluorescence. Nile Red is a cell permeable, lipophilic fluorescent dye that selectively stains neutral lipids, which constitute >97% of the lipids in Chlamydomonas lipid droplets. Listenberger, & Brown. Curr Protoc Cell Biol Chapter 24, Unit 24.2 (2007). Greenspan et al., J. Cell Biol 100, 965-973 (1985). Wang et al., Eukaryotic Cell 8, 1856-1868 (2009).

We collected images using a Nikon Eclipse Ti-E inverted fluorescence microscope equipped with a Photometrics Coolsnap HQ2 CCD camera, a 20×/0.75 NA air objective, a Sutter DG-4 Xenon arc lamp, and Nikon NIS-elements software. All filters are Chroma filters (Chroma).

We performed two basic types of quantitative microscopy-1) medium throughput screening (i.e., batches of 12-24 transformants for any particular cell line transformed with a given expression construct), and 2) low throughput quantification of selected candidates from the initial medium throughput screens, in which we affixed cells to the glass wells and washed before image collection.

To prepare cells for medium throughput microscopic screening, we put 40 μl of TAP into wells of 384-well glass-bottom plates (Arctic White, Inc.), and then deposited a small amount of cells (picked from 1-2 week old restreaks, onto selective media, of original colonies from a transformation) into the well using a sterile micropipette tip. We then waited ˜10 min for cells to settle and performed imaging.

To prepare cells for low-throughput measurement, we first grew cells in liquid media (TAP). We started cultures at a density of ˜1×10⁶ cells/ml from streaks on a plate, and then grew them for ˜5-7 days to saturation (˜1×10⁷ cells/ml). We washed cells and removed cell wall debris from the supernatant by centrifuging 1 ml of liquid culture for 5 min at 3,000×g, and resuspending in 1 ml TAP+0.5 μg/ml Nile Red. We then deposited ˜25 μl of resuspended cells at ˜1×10⁷ cells/ml and 100 μl TAP+0.5 μg/ml Nile Red into each well of a 96-well glass-sample plate that we had pre-treated for at least 15 minutes with concanavalin V (Sigma) (100 μl of 0.1 mg/ml in water). After letting cells settle for 10 minutes, we washed immobilized cells one time by evacuating the liquid in the well and gently adding 150 μl TAP+0.5 μg/ml Nile Red into the empty well.

For each well, we typically collected 5-7 image fields. We collected an in-focus brightfield image, two fluorescence images for quantifying Nile Red fluorescence—a FITC image (excitation filter ET490/20x, emission filter ET525/36m), and a custom Nile Red image (excitation filter ET490/20x, emission filter ET605/52m), and one defocused brightfield image for identifying cell boundaries. We extracted parameters of interest from images using Cell-ID 1.4 and analyzed them using the software package R and custom scripts. Chernomoretz et al Curr Protoc Mol Biol Chapter 14, Unit 14.18 (2008). and R Development Core Team, (2005).

Quantification of Fluorescent Protein Expression by Microscopy:

We quantified fluorescent protein expression essentially as described above (quantifying intracellular oil by microscopy), with the following differences. We used plain TAP to wash and resuspend cells, and we measured CFP (excitation filter ET430/24x, emission filter WR470/24m) or GFP (excitation filter S470/30x, emission filter S510/30m).

Localization of GFP-Tagged Secretion Proteins by Spinning Disc Confocal Microscopy:

We measured protein localization using spinning disc confocal microscopy. We used a Nikon Eclipse Ti-E inverted fluorescent microscope equipped with a Yokogoawa CSU22 spinning disk confocal attachment (Solarmere Technology Groyp), 405 nm and 491 nm lasers (Cobalt), and a photometrics Evolve EMCCD camera). We used either a 60×/1.45NA oil immersion Plan Apo objective or a 100×/1.4NA oil immersion Plan Apo objective. For measuring GFP, we used 491 nm laser light, a GFP long pass dichroic mirror (Chroma) and a ET525/50m emission filter (GFP channel). For measuring the chloroplast background, we excited with 594 nm laser light and used a 645/65 nm emission filter (RFP/mCherry channel).

We prepared cells for rapid screening and measurements in 96- and 384-multi-well glass bottomed plates as described above. We used TAP for washing and resuspending cells. We then analyzed images using ImageJ. Abramoff et al., Biophotonics International 11, 42, 36 (2004). To distinguish GFP fluorescence from autofluorescence, we compared GFP images to RFP(mCherry) images, which show chlorophyll and carotenoid fluorescence, the major sources of autofluorescence in Chlamydomonas. To estimated the contribution of autofluorescence in the GFP channel, we performed the following coarse unmixing procedure. We calculated the ratio of GFP to RFP signal in wild-type, untransformed reference cells. We then took GFP and RFP images of cells expressing GFP-tagged proteins. We estimated autofluorescence in the GFP channel by multiplying the RFP signal by the GFP:RFP ratio measured in reference cells. We then subtracted this from the total GFP signal.

### Example 2

Chlamydomonas Strain with Improved Transgene Expression.

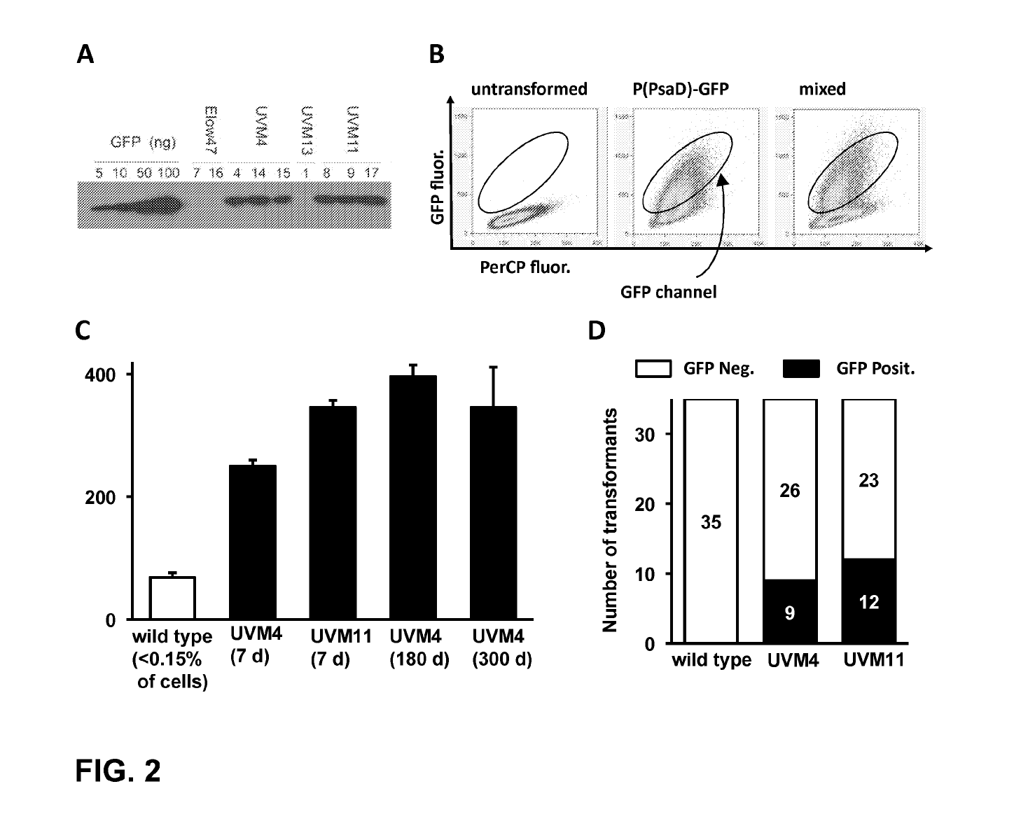

We transformed the non-mutant parental Elo47 strain and the mutant UVM4 strain with pJR38, which encodes GFP(C. reinhardtii codon-optimized) under control of the strong PsaD promoter. We measured GFP fluorescence greater-than 2 standard deviations above the mean fluorescence of untransformed cells in 15 of 72 randomly chosen transformants by epifluorescence microscopy. This ratio is a substantial increase over wild-type (Elo47) cells, which yielded only 0 out of 72 randomly chosen transformants that expressed GFP (FIG. 2).

Rapid Microscopy-Based Assay for Lipid Quantification Chlamydomonas.

We quantified intracellular lipids by fluorescence microscopy-based cytometry with a neutral lipid staining dye (Nile Red). Greenspan et al., J. Cell Biol 100: 965-973 (1985), and Listenberger & Brown, Curr Protoc Cell Biol Chapter 24, Unit 24.2 (2007). This measurement is a critical metric for manipulating TG amounts in the cells. For example, after transforming with an expression construct, we might find a bimodal distribution of cellular lipid content, which might result from a large amount of cell-to-cell variation in the activity of a particular promoter. In such a case, we can improve average lipid content more by using a less variable promoter than by using a stronger but equally variable promoter.

To stain droplets, we incubate cells in Tris-Acetate-Phosphate (TAP) growth media supplemented with 0.5 μg/ml Nile Red for 10 minutes, and then image cells by epi-fluorescence microscopy. We then quantify Nile Red staining by image-based cytometry. Gordon. et al., Nat. Methods 4, 175-181 (2007), and Chemomoretz et al., Curr Protoc Mol Biol Chapter 14, Unit 14.18 (2008). We observed lipid droplets in wild-type cells grown on selective media plates. Our visual estimates of average lipid droplet size (˜1 μm) and number of droplets per cell (2-3) is consistent with a more detailed analysis of C. reinhardtii lipid body quantity and size distributions in the cw15 strain, the parent strain of Elo47 and the UVM strains. Wang et al., Eukaryotic Cell 8, 1856-1868 (2009).

Increased Lipid Droplet Quantity by Expression of Transgenic DGAT2.

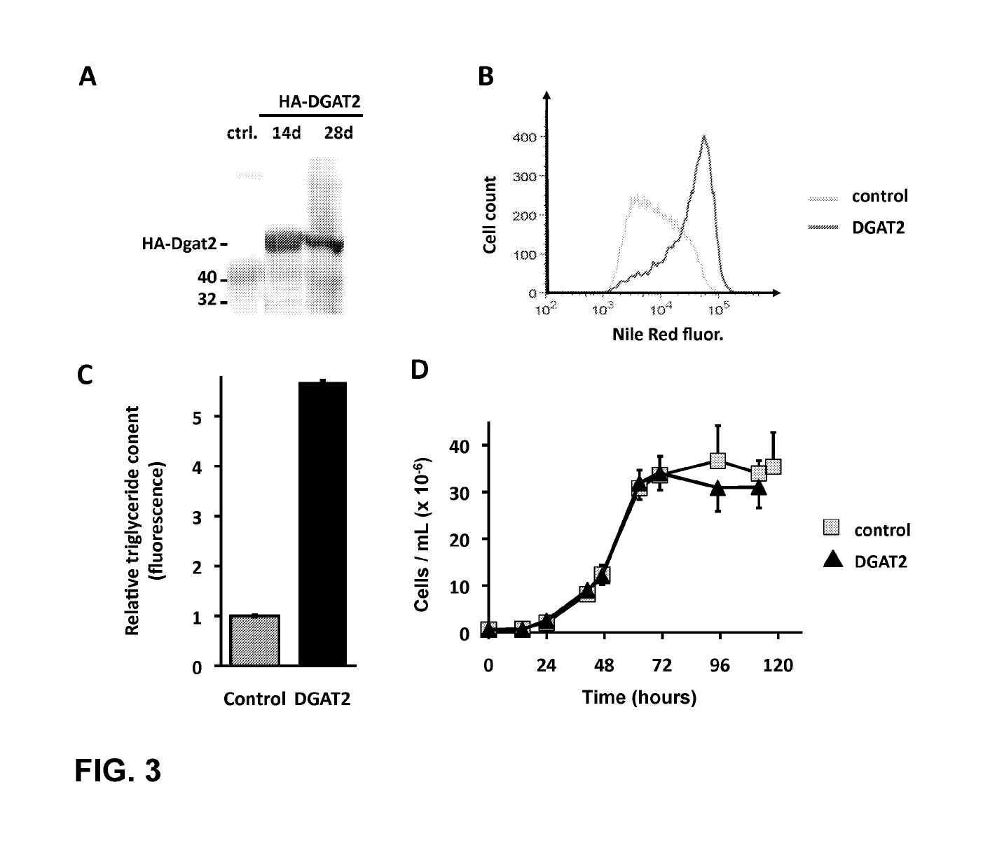

We expressed human DGAT2 (codon optimized for C. reinhardtii) under the control of the PsaD promoter (in vector pGPB1016 containing the paromomycin resistance cassette as a selectable marker) in UVM4 and wild-type cells. We randomly selected 24 paromomycin-resistant transformants, grew them on TAP-agar containing paromomycin, and screened them for increased lipids using our microscopic assay described above. We measured elevated Nile Red staining in four of 24 transformants of the UVM4 strain. We saw similar effects from expressing a 3X-HA tagged version of human DGAT2, as well as from expressing an untagged and 3X-HA tagged predicted C. reinhardtii DGAT2 ortholog (data not shown). We did not see elevated droplets in 24 transformants using a control vector expressing GFP. We calculated that cells expressing human DGAT2 and grown on selective solid media contained, on average, 2.8 times the lipid of untransformed wild-type strains (FIG. 3B). We saw a wide range of smaller increases in preliminary comparisons of these and other candidate cell lines in liquid cultures during different growth regimes (20-40%) increases in log growth).

### Example 3

Generation of Algae Strains that Synthesize Detectable Levels of C20:4.

Biosynthesis of large amounts of DHA and EPA in Chlamydomonas from existing abundant C18 FAs requires two types of enzymes, desaturases and elongases, to introduce double bonds at specific locations and extend the carbon chain length from 18 to 22, respectively.

First, C. reinhardtii codon-optimized versions of C. elegans FAT3 (CAA94233) and C. elegans ELO2. (CAB02921) are synthesized and expressed using vectors with strong promoters from either PsaD (Fischer & RochaixD Mol. Genet. Genomics 265:888-894 (2001)) or Hsp70A-Rbcs2 hybrid (Schroda et al. Plant Cell 11:1165-1178 (1999))) tagged with a small 3×HA epitope for immunoblotting and immunostaining

Second, UVM strains (UVM4 and UVM11) are transformed with FAT3 expression constructs, then are screened for highly expressing lines by qPCR, and assay for expressed protein by immunoblotting with anti-HA antibodies (Covance). Candidates of Fat-3 expressing lines are then assayed for increased C18:4Δ6,9,12,15 FA. Total lipids are extracted from cells with chloform/methanol (Griesbeck et al., Mol. Biotechnol. 34:213-223 (2006)) and the FA composition is analyzed by LC-MS/MS using services provided by the Kansas Lipidomics Research Center (KLCR). Five biological replicates are then assayed for each candidate transformant. C18:4 Δ6,9,12,15 producing cells (and wild-type cells as a control) are then transformed with ELO2 expression constructs, confirm expression of ELO2 as described above for FAT3, and assay for C20:4Δ7,11,14,17 production. A final, detailed FA and TG composition characterization are then performed of best-producing cell lines using services provided by the National Renewable Energy Laboratory (NREL).

Data Analysis:

For each candidate, the reported values are averaged for the desired C18:4Δ^{6,9,12,15} and C20:4 Δ^{7,11,14,17} for the five biological replicates, and the amount detectable is considered if the mean is at least 2 S.E. above the mean value reported for wild-type, untransformed cels.

The upstream precursor, C18:3 Δ^{9,12,15}, while known to be abundant in Chlamydomonas (16% of all FAs), is only present in a membrane-bound glycerolipid form (conjugated to diacylglyceryltrimethylhomoserine, or DGTS). To increase the amount of CoA-bound C18:3 Δ^{9,12,15}, which is the actual substrate for FAT3 and ELO2, yeast phospholipase (NTE1 or PLB1) is expressed in the algae to cleave C18:3 Δ 9,12,15 from DGTS. The free FAs produced by the exogenous phospholipase is conjugated to Co-A by native Chlamydomonas acyl-CoA acyltransferase activity. Riekhof et al., Eukaryotic Cell 4:242-252 (2005).

As a second strategy to increase C18:4 Δ^{6,9,12,15}, a major pathway that converts C18:3 Δ^{9,12,15} into C18:4 Δ^{5,9,12,15} coniferic acid (Riekhof et al., Eukaryotic Cell 4:242-252 (2005)) is blocked. RNAi is used to knockdown expression of Δ5 desaturase, which turns C18:3 Δ^{9,12,15} into coniferic acid. The expression knockdown is assayed by qPCR to confirm reduced levels of Δ5 desaturase mRNA, and assayed for reduced coniferic acid.

### Example 4

Maximization of Fatty Acid (FA) Synthesis and Storage in Triglycerides (TGs) in C. Reinhardtii by Expressing DGAT2 at Optimal Expression Levels

Identification of Promoters to Express Genes at Three Different Levels Over at Least Three Orders of Magnitude.

Candidate promoters (1 kilobase of 5′ UTR sequence) are selected based on microarray data measured by Stolc et al., Proc. Natl. Acad. Sci. U.S.A 102:3703-3707 (2005) that indicate gene expression levels in vegetatively growing cultures. At least 5 candidate promoters are picked for each of three expression levels (“high”, “medium”, and “low”) spanning approximately three orders of magnitude. In addition to these candidate promoters, and as references, two strong promoters, the PsaD promoter and the HSP70A-RBCS2 fusion promoter are tested and used A panel of constructs are then made in which each promoter controls expression of C. reinhardtii codon-optimized GFP. Fuhrmann et al., Plant J 19:353-361 (1999). The expression levels of each promoter are quantified in two ways: first, by detecting the level of gene transcription by qPCR; second, by measuring GFP fluorescence by our high throughput microscopic methods. (As an alternate method, particularly for higher expression levels, per-cell fluorescence levels are quantified by high-throughput flow cytometry).

Using these assays the tested promoters are re-classified into three strength classes-high, medium, and low-based on average level of expression that span at least three orders of magnitude. Within each strength class, at least one promoter is chosen. Amongst candidates with similar average expression, in order to have consistent expression levels in all cells of a population, promoters is picked with the lowest level of cell-to-cell variation in GFP expression, a quantity that is calculated from the cytometric methods.

For each of the final vectors, a sister panel of vectors is created with the paromomycin-resistance cassette replaced by the hygromycin B-resistance cassette to enable double-selection of two transgene expression cassettes. Berthold, et al., Protist 153:401-412 (2002).

Generation of Constructs to Express DGAT2 at Different Levels.

Both human and putative Chlamydomonas orthologs are tagged with a C-terminal 3×HA tag to facilitate immunodetection (the 3×HA tag does not interfere with DGAT2's TG-increasing function). We have demonstrated that transformation with expression constructs of human DGAT2, using a strong promoter (PsaD promoter), correlates with increased amounts lipids.

We identify transformants that express DGAT2 using our Nile Red cytometric assays, and confirm gene expression by qPCR and protein expression by immunoblotting using human DGAT2 antibodies (GeneTex) or anti-HA antibodies (Covance).

Identification of DGAT2 Expressing Lines that Maximize Lipid Production in Liquid Cultures.

We test which expression levels of which genes yield the highest FA and TG synthesis rates and accumulation levels while reducing the growth of the cultures the least (i.e., which candidates have the best total FA/TG synthesis rates and accumulation levels).

We directly quantify FAs and TGs as a % of total cell dry weight in house by TLC and HPLC after total extraction with chloroform/methanol, and using external services of KLRC for initial FA profiling and TG quantification, and of NREL for more detailed FA and TG characterization (see attached letters of support). Additionally, we use commercially available kits that indirectly quantify TGs by measuring the glycerol released after saponification or lipase treatment (Cayman Chemical). We perform at least 5 biological replicates for all measurements.

Data Analysis:

For qPCR measurements of gene expression, at least 5 biological replicates are measured and calculated for mean expression values and standard errors. For single-cell fluorescence cytometric data of GFP and Nile Red fluorescence from microscope images, Cell-ID is used or image processing and data extraction. Gordon et al. Nat. Methods 4, 175-181 (2007), and Chemomoretz et al., Curr Protoc Mol Biol Chapter 14, Unit 14.18 (2008). For data analysis of Cell-ID output and for standard flow cytometer total fluorescence outputs, the software package R is used (R Development Core Team R: A language and environment for statistical computing. (2005), at www.R-project.org). It is determined if average differences in fluorescence are significant by comparing distributions of wild-type and engineered cells and using Welsh's t-test (and using a significance threshold of p<0.01). For qPCR, FA, and TG quantifications, means calculated from 5 biological replicates of each sample is compared and considered for the difference significant if the mean value using the engineered cell line is at least 2 S.E. above the mean value unengineered control cell line.

### Example 5

Generation of Algae Strains that Secrete Increased Levels of Triglycerides

We constructed and tested a number of candidate secretion systems, each comprising a combination of lipid droplet-targeting domains and a plasma membrane, flagellar membrane, or flagellar lumen-targeting domain. The domains were either fused together to make a single polypeptide, or co-expressed with interaction domains that mediate non-covalent interactions. The protein or proteins additionally included a protein tag, either GFP or a 3×HA epitope tag, for microscope, flow cytometer, and immunoblot detection.

The genetic constructs were then transformed into UVM4 and/or UVM11. We screened for cell lines that expressed the proteins based on one or more of the following: 1) increased GFP fluorescence (if the protein or proteins contained GFP tags), 2) immunoblot detection (for either GFP and or 3×HA tagged protein(s)). We then measured total triglyceride levels in a well-mixed culture suspension, triglyceride levels in the media after separating cells, and triglyceride in separated cells.

Identification of a secretion construct that increases extracellular amounts of triglyerides: One candidate secretion construct increased the amount of extracellular oil. We engineered a fusion of the endogenous Chlamydomonas intraflagellar transport protein IFT20 (Lucker et al., 2005; Cole, 2003) and a lipid droplet-targeting domain from AAM-B demonstrated in the literature to target expressed GFP to lipid droplets in multiple species (Zehmer, 2008) (AAM-Bpep).

We first determined that AAM-Bpep localized to lipid droplets in Chlamydomonas. We made a construct to express AAM-Bpep-GFP, and transformed them into UVM4 and isolated transformants. We screened 12 transformants by spinning disk confocal microscopy, and in one transformant we observed annular intracellular distributions of GFP fluorescence consistent with the locations and sizes of lipid droplets (FIG. 15A).

We then transformed the IFT20-AAM-Bpep-HA (IAH) expression construct into UVM4. We screened 12 cell lines for expression of IAH by anti-HA Western blots. Four out of 12 cell lines tested expressed a protein with the predicted electrophoretic mobility (data not shown). We grew two of these cell lines (#2 and #7) in liquid media until saturation, and incubated them further for 7 days.

We then quantified extracellular and intracellular triglycerides and glycerol by measuring centrifuged supernatant and cell pellets (see Materials and Methods). The IAH-expressers contained a higher fraction of total oils in the extracellular space (FIG. 15B), consistent with engineered oil secretion in these strains. We concluded that the increased ratio of extracellular to intracellular oil is not due to cell lysis, based on two pieces of evidence: 1) the chlorophyll content of the supernatant in IAH-expressing cultures was not higher than that of control cultures, and 2) microscopic and flow cytometric analysis of total cell culture did not reveal excess cell debris in the IAH-expressing culture relative to control cultures (data not shown). The increased ratio results both from an increase in total extracellular oil (˜25% more, data not shown), but in also a lower total oil level (˜50% lower, data not shown).

We also expressed the secretion construct in our metabolically engineered Dgat2 strain for increasing the oil synthesis rates also increases the absolute levels of secreted oils.

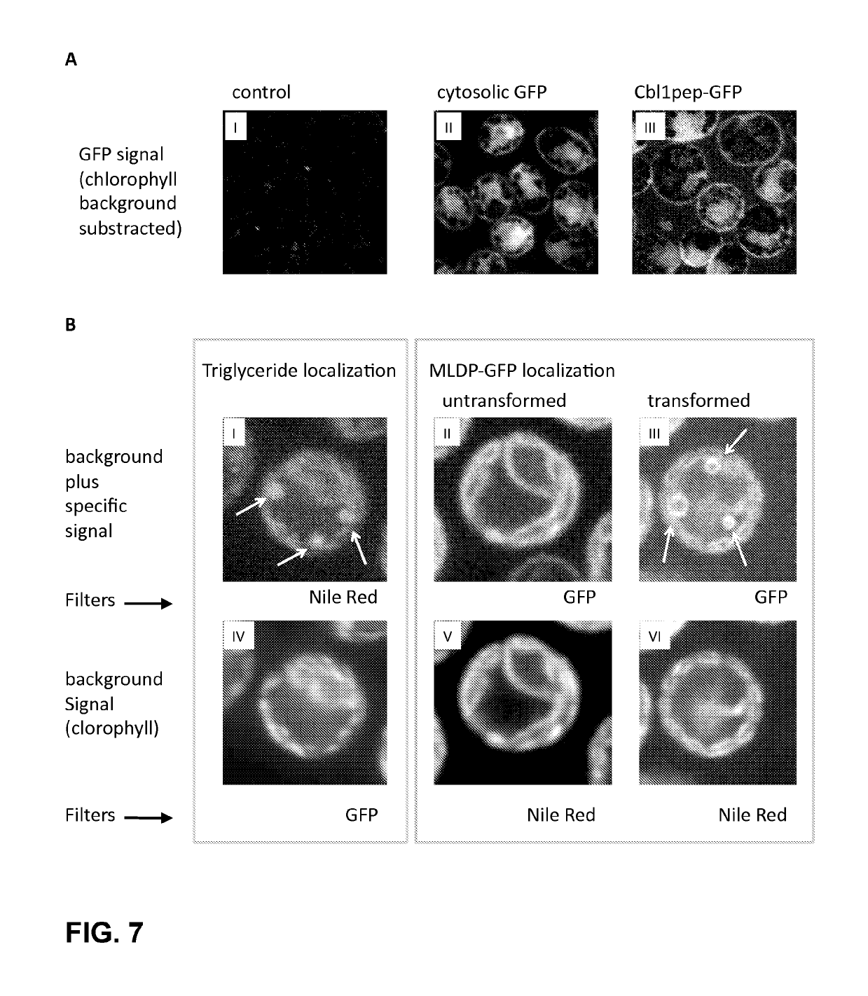

Additional lipid droplet targeting domain verified: In parallel to the work above, we expressed a number of other domains, tagged with GFP, in the UVM4 strain and observed their subcellular localization by spinning disk confocal fluorescence microscopy. One of these, Major Lipid Droplet associated Protein (MLDP), was isolated from purified lipid droplets and identified by mass spectrometry (Moellering 2010). In cells expressing GFP-tagged MLDP protein, we observed subcellular localization of fluorescence in the GFP channel indicating that MLDP-GFP was efficiently localized to lipid droplets (FIG. 7B). Preliminary analyses showed that MLDP-GFP cells had less GFP fluorescence in the cytosol than AAM-Bpep-GFP, suggesting that a tighter affinity of MLDP for lipid droplets than AAM-Bpep. IFT20-MLDP-HA is another viable candidate secretion construct.

1-65. (canceled)

66. A method for producing a lipid, comprising:
culturing a genetically engineered cell; and
producing a lipid secreted from said genetically engineered cell.

67. The method of claim 66, wherein said lipid is secreted in the form of a lipid droplet, fat globule, vesicle, or lipid droplet in a flagella or in a fragment of a flagella.

68. The method of claim 67, wherein said cell is transformed with and stably expresses genes that encode i) proteins that associate with the cell membrane, flagella, multivesicular bodies, or secreted exosomes, and ii) proteins that associate with lipid droplets, and that these protein fragments are either covalently attached or interact through protein-protein interactions

69. The method of claim 68, wherein said cell is transformed with and stably expresses one or more gene selected from the group consisting of: retroviral Gag protein (GAG), paramyxovirual Matrix protein (MA), acyl carrier binding protein (ACB1), butryophillin (BTN), syntaxin (STX), flagellar membrane glycoprotein (FMG1-B), calcineurin B-like protein (Cbl1), multicopper ferroxidase (Fox1), intraflagellar transport protein 20 kDa (IFT20), Arl13, intraflagellar transport protein 27 kDa (IFT27), Rab8, adipophillin (ADPH), perilipin, xanithine ornithoreductase (XOR), major lipid droplet binding protein (MLDP), and AAM-B, or a fragment thereof.

70. The method of claim 69, wherein said cell is an alga cell or a yeast cell.

71. The method of claim 66, wherein the genetically engineered cell is engineered by over-expressing a di-acylglycerol acyltransferase 2 (DGAT2) or inhibiting a gene in the starch synthesis pathway.

72. The method of claim 68, wherein the expression of said genes gene is stable for at least 9 months on solid media.

73. The method of claim 71, wherein the DGAT2 gene has an amino acid sequence that is at least 90% identical to an amino acid sequence selected from the group consisting of SEQ ID NOs: 11-50.

74. The method of claim 71, wherein the gene in the starch synthesis pathway is STA1 or STA6.

75. A method for producing a lipid in a cell, comprising:
culturing a metabolic engineered eukaryotic microalgae cell to produce a lipid.

76. The method of claim 75, wherein said metabolic engineering is selected from the group consisting of:
over-expressing a di-acylglycerol acyltransferase 2 (DGAT2) gene in said cell; and
inhibiting a gene in the starch synthesis pathway in said cell.

77. The method of any one of claim 76, wherein the expression of said introduced gene into the nuclear genome is stable for at least 9 months on solid media.

78. The method of any one of claim 77, wherein said cell is grown under a condition where the cell growth rate is affected less than 25% in comparison of a cell that is not metabolic engineered.

79. The method of claim 78, wherein said DGAT2 gene has an amino acid sequence that is at least 90% identical to an amino acid sequence selected from the group consisting of SEQ ID NOs: 11-50.

80. The method of claim 78, wherein said gene in the starch synthesis pathway is STA1 or STA6.

81. A method for producing DHA, comprising:
culturing a genetically engineered eukaryotic microalgae or Chlamydomonas cell to produce DHA.

82. The method of claim 81, wherein said cell is transformed with an elongase gene and a desaturase gene.

83. The method of claim 82, wherein said cell is transformed with a Δ6-desaturase gene, a Δ6-elongase gene, a Δ5-desaturase gene, a Δ5-elongase gene, and a Δ4-desaturase gene.

84. The method of claim 82, wherein said production of DHA is by the expression of one or more genes selected from the group consisting of: Fat-3, Elo-2, Fat-4, elo, and IgD4.

85. The method of claim 82, comprising converting naturally occurring C18:3 (18:3Δ9, 12, 15) to C20:4 (C20:4Δ8,11,14,17).


SEQUENCE LISTING (61 sequences)

SEQ ID NO: 1 - DNA, 396 bp, Artificial Sequence
  Description of Artificial Sequence Synthetic promoter sequence
ccacgagtgaccagcgaaacttgtaaattgaatattgtatcctatatgtatgcgcctagaccatttttcagcttttgggaggggtaccgagtgtcgggctttcgtggccactcgcgctcctaatgcatgcatgcaccggcctggtcgaggtgcctattgagtgtctcaactcagaccattttgatagtttctatttactcatacaaatggctctgtcagcgcagcagactcgccaggtggcctcggctcgttcctcgcgcaagggtaggcccagaatgtgcaagcttctagcaactttttcgccactccctgaccacgcaattttgctccttgcagccttcgcgcctgctcgcatctctcgcacccgtgctgccgtggtcgtgcgcgcggaggccc

SEQ ID NO: 2 - DNA, 291 bp, Artificial Sequence
  Description of Artificial Sequence Synthetic promoter sequence
tggccaacttgagcgcatctagcagctgtacacgcacggtccgcgtcagctcgcaatggaactctggttccagcagcctgggcatgtctgataatagacactcgttgatatgcaataatcacttgtcacattaggcattagtagcggtgctggctggcgggccctaggcgactgttcagtaacgctgtcgcaaccacatcttcggctttgcacctctgcacctttactctccgcttgttccctcatcagccacctttcacaatgccctcactgacgactagcagcagcgca

SEQ ID NO: 3 - DNA, 557 bp, Artificial Sequence
  Description of Artificial Sequence Synthetic promoter sequence
actgcagtgcgtaacaaggtcgtcaagaggggaatgtcattcgtctcaggtgaagctgaaggtatggaggcacatgagcgggtgcgggtgcggagcagcggctgcgcgagtaggtcatgtcgagcgcgcgctcgtacagacggtactgtgtattctcatgtagcttaggtgaagccccggctttcgaatgcggggaagcggcaggcgagggatggcgactaccactgcggagtgaggatagatgccggcaggtgcgggccgcaccggcctgcccgcatacggcttgcccggagggatttggattttttcggagaagcgcataggcgccatttgtgtagcatgtagtatgtgccaccggcacgccgcatgccgatgtacggtggaggcgtgaggaggcgtgaggtggagggcatgggaggtgaaatgacgctgctcatgcaatcccagcgcgcctttccatccttaccctaagttgttgtcccccatccagtgagcattggcacaaaagtgctgcagtagacaaaccttcctttgagttgtactaggtgcacgaaaac

SEQ ID NO: 4 - DNA, 625 bp, Artificial Sequence
  Description of Artificial Sequence Synthetic promoter sequence
tccgcgaatcaatgcaatcccgcattgcagcgtcctccccaacaagtcccccccctgcccagccccccagctgctcgccagtggggcgcgcgcggcctcatgagcggccgctgcagagcccattctcccgcctcctgtccacctggcagacacgtaccacagcgggcccagctccctcccgtccctcaccactgcgccctcaggccctcccgctcccgttcctgctcccgtttcggccgcggccagcagcagcagcacccggtggatggcgccgcccgccgcggacagccactgctcgcctgggggcgggggcataactggtgggtggttaacgacaagtattgtgcgaagtgctgcgctattctgggcccgctgagacagcttgcgtggaactggttgcctcgaacccgtgcacagccgcaaccatcccccgctttttgaagccgctgttgtggcacctcctttgcaatcggcacgactgcgttaaggctgccccgaggccccccccaccaccccctccccggtcgggacttttcatgtctacgtcactgtgggaataaaacaggggcgggttagtattatttgcaatttgctgcacaatttgtgtctgtcaggtacgccttgt

SEQ ID NO: 5 - DNA, 475 bp, Artificial Sequence
  Description of Artificial Sequence Synthetic promoter sequence
catctaattggcgctgttgctgggtatgaagttagcgctacactcagcccgatgaagagtttatgacgagcgacggttcagatgcgttccggagctggaccaccgtcacgcagagccccaacaaaggctccacatgtgctaatgcaaacaacatgtatcacttgtaaccagcagctttgccaatgtgcatgatttggcttagcgaggcagtcgcaagcctggcgcgtggcgcattcttgcagcgcgccagcccccgcagctttagcggtccattgcaagctcaatgggccacgtcgcaatgcaccagcgggcgagcggacagcgcacgctggcgcgcgaccgatcgggcgcggggtcgatcgagatggccgggtacggcgatcggcgcgcgagatgggaccatgaaccccttcttaagcgacgcgcgctcgtgaaaatatcgtgaccggcttcaaccagtccccacacatcagtc

SEQ ID NO: 6 - DNA, 576 bp, Artificial Sequence
  Description of Artificial Sequence Synthetic promoter sequence
tggagattgcgcccagtttacaccgggcacgtgcattatacagtgcgcccaggtcctgccgggtccgtgttgcggtgctgtcacggcgtgacagccctcccctgcagcccgacccccacgccatccagcgccagcgcgccatctatcgtgactactgggcctgaccggcctgcgcccgtgcattagtcgtaatgctcctcttgaccgcatctgtagggaaaggaccaagctgcgaccagcgtgagcccgggcctaacatgccgggcgcccatggcacggttatgctccggaaggttgtaccagcggcctgagctactaagtggaacctgagaatggtttcggaagctccaaacacagccgggctaggcccgaccggtattaggtgatgggcgttcgcgatcgagctttcggcgcgcattaatacgctgtcggcgaggagggaggttttgtaaagacgtttatttatatgcgcttagattatcagacctctcgcgtgtggtgtttcacggtcacgacactaagcctccggaatactaagacccccgtgatacttagccctcgtttcctctggggacg

SEQ ID NO: 7 - DNA, 358 bp, Artificial Sequence
  Description of Artificial Sequence Synthetic promoter sequence
tagtatcctacgccgtgttgcagcactgtgtacaactgaattggtcgaagagcgaggggagcgtcctacagggagttgtcgggtgtcgcgacagttccaccctttcagtatggaccctagcgcgtgttataaatttatgtctacagagcaatgagtttgattgagatgtagccacgttcttcttccgctcgcgtgccggtcggccatcgagggcttctcatgatgcagcgttttctgtccccttcccccacgacctgttttgctgtgatccagcgccggcaccctattcctttcatagatctaggacctgaaatttacgttgccatagtgcttgactgctgctctgccaggtgcactg

SEQ ID NO: 8 - DNA, 411 bp, Artificial Sequence
  Description of Artificial Sequence Synthetic promoter sequence
ccatgcaaacgtgtcacttggatgtttgcggtgctgcacgcgccttcgatttggcgtttggtgcattcttacgcaaccaggcgaagacaggtgaaggcgggttccgggcgaaagaaccacccgtgtaccaccttaattaattaaaataaatgtttcagaatgcatcgaatcatccagcagtcgtgttcaagacctaaacatcgcgaaatctttgcacgcattacgcacataaatggtctcattaggggtgacctacggaatgtggattggtaattgtgaccaatatgaggaacgcgtggcccctcgcccctgggccgtgctccccatccggtcccaggatcctgcaacttttccacaggcaggcccctagcggtgacatttgaatcttactaaatgaacataatgttgagc

SEQ ID NO: 9 - DNA, 468 bp, Artificial Sequence
  Description of Artificial Sequence Synthetic promoter sequence
accggttatgagcagggattcaccccttggcataaggcggcctacatcatacgcgctttctcatcaagggagccatgccctggccggacatagtgcaccgtgcaagctgcggaaaatgccgtgatttcaatatgttggagatttccggcagatgaccaggagctaggaagtattagcaatcgccgctgaccggcggagagtccggcgcggtcgcactgttttgtgctgacgcagggttcgtgccggggggtctccacgttgcttggggccctcaggctgtatagtttagttcattgggtatgaaatagctggaagcatgcagatgggtgaagggcacggtttaccagtttagttttctgaatccgccctggagtcattccgcccgctcctgcgacttcagcgagcgactcgccccttgctctttctcttcctggttgcgtgtctagcctgacactgcttcccctcgca

SEQ ID NO: 10 - DNA, 604 bp, Artificial Sequence
  Description of Artificial Sequence Synthetic promoter sequence
caggaaatgcagagcaggataggccacggccggtgacacgctggcgcgacccgtagccagtaggccgcggctgactcctagagccctggcctcccgcagggctgctagcatccagcgggcggcgatggcttgcgatgtcaggcgtgcggcaggagtcggctgagggtggccagcgaggtgtgccacaacttcggggcgcccgacctaaggtgagggcacgccctgcccgtcggattgtccctaagcgatcacgaattgtatattgatttgaacagttactgacacggagctgagggtaattgccgttcagtggtgacgacttgtgagctgctcgtgcagccccagttttctgctgatgaagcagctaatctcctgaacccgaaaaccctagagttgagagcaaagacagctccctgaattgacacataatctgcatgggcgccgggtcgtattttgttgtacagccgcgtcgtggcgcgctcttgcatattagctccgcactttcactcaagttcgcatagttgcctgtgattagtagggagcctcacttgaccttacaaagcccacggacacaacccgtgtggccaactcgcgccaaattaca

SEQ ID NO: 11 - protein, 388 aa, Mus musculus
MKTLIAAYSGVLRGERRAEAARSENKNKGSALSREGSGRWGTGSSILSALQDIFSVTWLNRSKVEKQLQVISVLQWVLSFLVLGVACSVILMYTFCTDCWLIAVLYFTWLAFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVNRDTIDYLLSKNGSGNAIIIVVGGAAESLSSMPGKNAVTLKNRKGFVKLALRHGADLVPTYSFGENEVYKQVIFEEGSWGRWVQKKFQKYIGFAPCIFHGRGLFSSDTWGLVPYSKPITTVVGEPITVPKLEHPTQKDIDLYHAMYMEALVKLFDNHKTKFGLPETEVLEVN

SEQ ID NO: 12 - protein, 388 aa, Rattus norvegicus
MKTLIAAYSGVLRGERRAEAARSENKNKGSALSREGSGRWGTGSSILSALQDIFSVTWLNRSKVEKHLQVISVLQWVLSFLVLGVACSVILMYTFCTDCWLIAALYFTWLAFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVNRDTIDYLLSKNGSGNAIVIVVGGAAESLSSMPGKNAVTLRNRKGFVKLALRHGADLVPTYSFGENEVYKQVIFEEGSWGRWVQKKFQKYIGFAPCIFHGRGLFSSDTWGLVPYSKPITTVVGEPITVPKLEHPTQKDIDLYHTMYMEALVKLFDNHKTKFGLPETEVLEVN

SEQ ID NO: 13 - protein, 616 aa, Pan troglodytes
MYSYKNDPPTNLSSALAQEDGSDQVPAHQGAFPQCAGGIRPNARLQCIPRPRACYSVQREFQTVFHRALEIGCVGWASVCFFPQNAPKEQELYPQHPAQGWGVSGKVGQIEGQMRNECTGSSLCNIPSPTLTPPPNSCREKALRELSDKVLRSGTPAPGLPPLLGSRLFLSRHHWPPAAAPGVLAAQPPRRPGTPVLCAKPWPRGPGHGPGARGEAASRGAVTGRASAMKTLIAAYSGVLRGERQAEADRSQRSHGGPALSREGSGRWGTGSSILSALQDLFSVTWLNRSKVEKQLQVISVLQWVLSFLVLGVACSAILMYIFCTDCWLIAVLYFTWLVFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVSRDTIDYLLSKNGSGNAIIIVVGGAAESLSSMPGKNAVTLRNRKGFVKLALRHGADLVPIYSFGENEVYKQVIFEEGSWGRWVQKKFQKYIGFAPCIFHGRGLFSSDTWGLVPYSKPITTVVGEPITIPKLEHPTQQDIDLYHTMYMEALVKLFDKHKTKFGLPETEVLEVN

SEQ ID NO: 14 - protein, 388 aa, Homo sapiens
MKTLIAAYSGVLRGERQAEADRSQRSHGGPALSREGSGRWGTGSSILSALQDLFSVTWLNRSKVEKQLQVISVLQWVLSFLVLGVACSAILMYIFCTDCWLIAVLYFTWLVFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVSRDTIDYLLSKNGSGNAIIIVVGGAAESLSSMPGKNAVTLRNRKGFVKLALRHGADLVPIYSFGENEVYKQVIFEEGSWGRWVQKKFQKYIGFAPCIFHGRGLFSSDTWGLVPYSKPITTVVGEPITIPKLEHPTQQDIDLYHTMYMEALVKLFDKHKTKFGLPETEVLEVN

SEQ ID NO: 15 - protein, 569 aa, Macaca mulatta
MYSYKNDPPTNLFSALAQEDGSDQVPAHQGAFPQCAGDIRLNAHLQCIPQPSTCYSVQREFQTVFHRALEIGMPLGSKNCTHSTQHKYGGVSGKVGQIEGQMRNEPPPSQSLLRRRLFLPRRLWPTGRPAAAPGVLATQPRRRPGAPVLCAKPWPRRPGHGPGARCEAASRGAVTGRASYSAMKTLIAAYSGVLRGERQAEADRSQRSHGGPALSREGSGRWGTGSSILSALQDLFSVTWLNRSKVEKQLQVISVLQWVLSFLVLGVACSAILMYIFCTDCWLIAVLYFTWLVFDWNTPKKGGRRSQWVRNWAVWRYFRDYFPIQLVKTHNLLTTRNYIFGYHPHGIMGLGAFCNFSTEATEVSKKFPGIRPYLATLAGNFRLPVLREYLMSGGICPVNRDTIDYLLSKNGSGNAIIIVVGGAAESLSSMPGKNAVTLRNRKGFVKLALRHGADLVPMYSFGENEVYKQVIFEEGSWGRWVQKKFQKYIGFAPCIFHGRGLFSSDTWGLVPYSKPITTVGKPLACPRLEHPTQQDIDLYHTMYMEALVKLFDKHKTKFGLLETEVLEVN

SEQ ID NO: 16 - protein, 361 aa, Bos taurus
MKTLIAAYSGVLRGTGSSILSALQDLFSVTWLNRSKVEKQLQVISVLQWVLSFLVLGVACSVILMYTFCTDCWLIAVLYFTWLVFDWNTPKKGGRRSQWVRNWAVWRYFRDYFPIQLVKTHNLLTSRNYIFGYHPHGIMGLGAFCNFSTEATEVSKKFPGIRPYLATLAGNFRMPVLREYLMSGGICPVNRDTIDYLLSKNGSGNAIIIVVGGAAESLSSMPGKNAVTLRNRKGFVKLALRHGADLVPTYSFGENEVYKQVIFEEGSWGRWVQKKFQKYIGFAPCIFHGRGLFSSDTWGLVPYSKPITTVVGEPITIPRLERPTQQDIDLYHAMYVQALVKLFDQHKTKFGLPETEVLEVN

SEQ ID NO: 17 - protein, 348 aa, Ailuropoda melanoleuca
GTGSSILSALQDLFSITWLNRSKVEKQLQVISVLQWVLSFLVLGVACSAILMYTFCTDCWLIAVLYFTWLAFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVNRDTIDYLLSKNGSGNAIIIVVGGAAESLSSMPGKNAVTLRNRKGFVKLALRHGADLVPTYSFGENEVYKQVIFEEGSWGRWVQKKFQKYIGFAPCIFHGRGLFSSDTWGLVPYSKPITTVVGEPITIPKLEHPTQQDIDLYHAMYMEALVKLFDKHKTKFGLPETEVLEVN

SEQ ID NO: 18 - protein, 369 aa, Canis lupus
MTSSQEEVRDTTGGQGCEEGQGTGSSILSALQDLFSITWLNRSKVEKQLQVISVLQWVLSFLVLGVACSAILMYTFCTDCWLIAVLYFTWLAFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVNRDTIDYLLSKNGSGNAIIIVVGGAAESLSSMPGKNAVTLRNRKGFVKLALRHGADLVPTYSFGENEVYKQVIFEEGSWGRWVQKKFQKYIGFAPCIFHGRGLFSSDTWGLVPYSKPITTVVGEPITIPKLEHPTQQDIDLYHAMYMEALVKLFDKHKTKFGLPETEVLEVN

SEQ ID NO: 19 - protein, 361 aa, Sus scrofa
MKTLIAAYSGVLRGTGSSILSALQDLSAITWLNRSKVEKQLQVISVLQWVLSFLVLGVACSVILVYLICTDCWLITALYFTWLAFDWNTPKKGGRRSQWVRNWAVWRYFRDYFPIQLVKTHNLLTTRNYIFGYHPHGIMGLGAFCNFSTEATEVSKKFPGIKPYLATLAGNFRMPVLREYLMSGGICPVNRDTIDYLLSKNGSGNAIIIVVGGAAESLSSMPGKNAVTLRNRKGFVKLALRHGADLVPTYSFGENEVYQQVIFEEGSWGRWVQKKFQKYIGFAPCIFHGRGLFSSDTWGLMPYSKPITTVVGEPITIPKLEHPTQQDIDLYHAMYTEALVKLFNQHKTKFGLPETEVLEVN

SEQ ID NO: 20 - protein, 392 aa, Monodelphis domestica
MKTLIAAYSGVLRGEDQAESAPGSPGYLEMPLDWGTGIPARAGRCTGSSILSALQDLFSIPWLNKSKVEKQLQVISVLQWVLSFLVLGITCSLIFMYIFCTDCWLISVLYFTWLIFDWNTPKKGGRRSKWVRNWAVWRYFRDYFPIRLVKTHNLLTTRNYIFGYHPHGIMGLGAFCNFSTEATEVGKKFPGIRPYLATLAGNFRVPVLREYLMSGGICPVNRDTIDYLLSKNGSGNAIIIVVGGAAESLNSAPGKNAVTLKNRKGFVKLALRHGADLVPIYSFGENEVYKQIIFEEGSWGRWVQKKFQKYIGFAPCLFHGRGFFSSNTWGLVPYSKPITTVVGEPITIPKLEHPSQKDIDLYHGMYMESLVKLFDKHKTKFGLPETEVLEVN

SEQ ID NO: 21 - protein, 488 aa, Equus caballus
MKTLHSRLLRGPASEHRDQAARKKSSDGDLGDEPSRRGSAGRGAFILGLCLDSSRRSWIMTKEDTESQTGEVAFPVSQQAGAPPPGDAGGEDSGQGSGHMDEPGTSPGTQELLAEEGDQILKKRKGLSVSPEAPAPSPPSSTGSSILSALQDLFSISWLNRSQVEKQLQVISVLQWVLTFLVMGVSCSVILLYTFCTDCWLIAVLYFTWLVFDWNTPKKGGRRSQWVRNWAMWRYFRDYFPIQLVKTHNLLTTRNYIFGYHPHGIMGLGAFCNFSTEATEVSKKFPGIRPYLATLAGNFRMPVLREYLMSGGICPVNQDTIDYLLSKNGSGNAIIIVVGGAAESLSSMPGKNAVTLRHRKGFVKLALRHGADLVPIYSFGENEVYKQVIFEEGSWGRWVQKKFQKYIGFAPCIFHGRGLFSSDTWGLLPYPKPITTVVGEPITIPKLEHPTQQDIDLYHAMYMEALVNLFDRHKTKFGLPETEVLEVT

SEQ ID NO: 22 - protein, 358 aa, Taeniopygia guttata
MKTIIAAYSGVLRGTGSNILSALQDLFWLLKSKAEKQLQIISVLQWVLTFLIMGIACTLILMYILCTDCWAIAALYLAWLVFDWNTPKKGGRRSQWVRNWAIWRYFRDYFPIRLVKTHNLLTTRNYIFGYHPHGIMGLGAFCNFSTEATGVGQKFPGIRPYLATLAGNFRMPILRDYLMSGGICPVNRDSIDYILSKNGSGNAIIIVVGGAAESLNCTPGKNSVTLKNRKGFVKLALRHGADLVPVYSFGENEVYKQVIFEEGSWGRWVQKKFQKHIGFAPCIFHGRGLFSSNTWGLLPYSKPITTVVGEPITIPKIDNPSQKDVDFYHSMYVDSLIKLFDKYKSKFGLPETEVLEVN

SEQ ID NO: 23 - protein, 361 aa, Xenopus laevis
MKTIIAAYSGVLRGTGSSLLSAVHDLPSIPWLSKSSVVRHLQIISVLQWVLSFLILGVACTAVLVYIFCTDLWLIAALYFTWMVLDWNTPYKGGRRSSWVRNWAVWRYFRDYFPVKLVKTHNLLPSRNYIFGYHPHGIMCLGAFCNFGTEATGVSKKFPGIKCHLATLAGNFRMPVLREYLMSGGICPVNRDTINYILSKNGTGNAVVIAVGGAAESLNCRPGKNTVTLLHRKGFVKVALQHGADLVPIYSFGENETYKQVVFEEGSWGRWIQQKFQKYVGFAPCLFHGCSFFSSNSWGLVPYANPITTVVGEPITVPKIEQPTQKDVELYHSMYLSSLHRLFDKYKTKLGLPDSETLEFI

SEQ ID NO: 24 - protein, 361 aa, Xenopus tropicalis
MKTIIAAYSGVLRGTGSSLLSAVHDLPNIPWLSKSSVVRHLQIISVLQWVLSFLILGVACTAVLVYIFCTDLWLIAALYLTWMVLDWNTPYKGGRRSSWVRNWAVWRYFRDYFPIKLVKTHNLLPSRNYIFGYHPHGIMCLGAFCNFGTEATGVSKKFPGIKCHLATLAGNFRMPVLREYLMSGGICPVARDTIDYILSKNGTGNAVVIAVGGAAESLNCRPGKNTVTLKQRKGFVKVALQHGADLVPVYSFGENEAYKQVVFEEGSWGRWIQKKFQKYVGFAPCLFHGCSFFSSNSWGLVPYANPITTVVGEPITVPKIEQPTQKDVELYHAMYVTSLQRLFDKYKTKLGLHDSEMLEIV

SEQ ID NO: 25 - protein, 314 aa, Arabidopsis thaliana
MGGSREFRAEEHSNQFHSIIAMAIWLGAIHFNVALVLCSLIFLPPSLSLMVLGLLSLFIFIPIDHRSKYGRKLARYICKHACNYFPVSLYVEDYEAFQPNRAYVFGYEPHSVLPIGVVALCDLTGFMPIPNIKVLASSAIFYTPFLRHIWTWLGLTAASRKNFTSLLDSGYSCVLVPGGVQETFHMQHDAENVFLSRRRGFVRIAMEQGSPLVPVFCFGQARVYKWWKPDCDLYLKLSRAIRFTPICFWGVFGSPLPCRQPMHVVVGKPIEVTKTLKPTDEEIAKFHGQYVEALRDLFERHKSRVGYDLELKIL

SEQ ID NO: 26 - protein, 309 aa, Physcomitrella patens
MAEVGGNVKLWSIIAMVMWLGGFHINFIVGILCLVHLPSPWAITILAIWVTLMFLPAEYDTPMGSKVARFIVTHAKNYFPIKVIFEDESAFDPNQSYVIAAEPHSVLPIGIVVLTPQSGILPVNNLRALASSAVFWSPVVRHIWTWLGVAPVSRKSFTAFLQKGISCIVVPGGVQECLFMEDHREVVFLKQRYGFVRIAMEAGSPLVPTFCFGQRNAYKWWKPTGKWYNQLSRAIGFTPLVFWGRYGGPVPYRTPMYYVVGKPIPVPKTTNPSQEEVSVILGQFIEALEALFEKHKGSLEFEDDTLLVY

SEQ ID NO: 27 - protein, 313 aa, Physcomitrella patens
MAGGDLKKGFKYDVLTFLAIVVWMGGLHLNVLVVGICLANLRSHWALTLLAFWIALIFIPVRPNGGLGQAVARYICKYAPAYFPIKVVFDDENAFDSKKSYVFAAEPHSLLPIGIIALQPLSGNLPVPNLRALATSAVFWTPVVRHVWSWLGVSRVTRKFMTEFLQKGTSVIIIPGGVQECLYMERGREVVYLKKRYGFVKVALETGSLIVPTFCFGQTNCYKWWKPTGQWYSRLSRRIGFTPLVFWGRYGTPIPFRTPMTYVIGKPIEVVRNPNASHEEVAKVLEQYIEAVERLYENHKAALGFKDSPLFVY

SEQ ID NO: 28 - protein, 390 aa, Physcomitrella patens
MGSLDNTSRIARQELEVAHKTVNDDRSKSGVTSNQGDGGSVPQFHYFCKPHSTTVGTVLVLIFYHMLPFTAIPHILLFMYLPYYLQQRCSYAGLTIYVGFLLSLLVVPPYYSKTVRRKVRVLYEALAVHLPSARFIIVPKEPLPQDKGYIFAIHPHGRMFYSNALFSQLHEIWRAPLKLTHGDLFQTAAGGFFYAPFARNWFYMIGIMPASKENIVDKLRNKDHVTIAVGGVREVCLGTQVDADVLYIKRRRGFLQIAMDEGAGVVPVYAFNENQLFKHDPRFLLDFWQWVNNYVKIGVPFMRGYFNLPMPYSKELLLVFGEPLFSKEDESIEDFHARYVESLTQLFESGRTDNTGSHRNTDLRERMPTIDARDVGSTWLEAHCMKPALP

SEQ ID NO: 29 - protein, 363 aa, Physcomitrella patens
MGSLGGESVVVNPELNGEAKVVTDVQSGGASLKQGEGKDDAPKIHYFARPNSTTLSTVVVIFLYHMLPFTALPNIGLLYFPFSLYKRSEYVKLTLYLGLMLVLVVMPPHYSKTLRRKVRWLYEELAVYLPSAKFVIVPQEPLPQGKGYIFAIHPHGRMFYSNAMFSQLHEIWRAPLGLTHGDLFQTAAGGFFNVPIARNWFYSIGVMPVTKKNIVTKLRNKDHVTIAVGGVREVCLGTDNEADSLYLKNRRGFLRIAMDEGAGVVPVYAFNENQLFKHDPKAVLNFWQCVNKYVKIGVPFMRGIWNLPMPYRKEILLVFGDALFAEEGESIEDFHGRYIDNLRQLFDKYVKYSPDPNHKLIIC

SEQ ID NO: 30 - protein, 332 aa, Zea sp.
MGADGGLNRAAEKPRDGEGGEARVFRCTDYSLPRTTLALALWLGGIHFNVLLVLASLFLLSGRAAAIVVAFQLLFMFAPVNDRDKWGQSVARFICRHAMGYFPITLHVEDYKSLDPSRAYVFGYEPHSVLPIGLSALADLVGFLPLTKIKVLASSAVFYTPFLRQIWTWLGLVPATRKNFYCYLGAGYSCIVVPGGVREMLHMNNDSEVAFLKSRKGFVKIAIQSGCPLVPVFCFGQSYAYKWWRPGGKLFIKIARAVKFTPIIFWGRFGTPFPFPKPMHVVVGKPIEVNKIPHPTIDEINEVHEQFIIAMRDLFEKYKAKAGYPGLHLRVL

SEQ ID NO: 31 - protein, 317 aa, Glycine sp.
MRKVFNGVEEFSESRNVFKTVPALVLYLGAIHFNLALILWATVFLPLSKGLLVFGLLLVFVLIPVDENSIFGHKLSKYICKHICSYFPITLHVEEAKAFRPDQAYVFGYEPHSVFPIGIVALGDSTGFMPLAKTKFLASSAVFYIPFLRHIWTWLGFTPVTKQNFISSLEAGYSCILVPGGVRETFFMEPGCEIAFLKQRRGFVRIALQMGLPLVPVFCFGQTKAYKWWKPPGRLMQNLARFLKIIPLFFWGIYGSPIPFKNPLYIVVGRPIELEKNPEPTMEQVAKVHSQFVEALQDLFDRHKAHAGYTNLELKIF

SEQ ID NO: 32 - protein, 338 aa, Oryza sp.
MGAGNGEVRYGGGGAGAGEAAVMADDGTTVFRGTAQPPVRTTVALALWLGAIHFNAFLLLASLFLFPRRVAAMVLATQLFFMFAPVNDMSRLGRKIARFISKCVIGYFPVTLHVEDYKAFDPNRAYVFGYEPHSVLPIALGVLLELVGFMPLPKIKVLASSAVFYTPFLRQIWTWLGLVPASRKNFYSYLKAGYSCVIVPGGVQEMLHMDHDSEVAFLKSRKGFVKIAMETGSPLVPVFAFGQSYVYKWWRPGGKLIVKIARAIKFTPIMFWGKFGTPIPFATPMHVVVGRPIEVKKNAQPTFDEINEVHEQFVVALQELFEKYKTKAGYPNLHLRVL

SEQ ID NO: 33 - protein, 328 aa, Picea sp.
MEGEIRHEQILDRPEPTRIVSSSQSRYGFRSLVALIMWLGTIHFDAILVLLSLIFLPSTWAFLVLGSLVILMVIPVDENSKWGNRLARFICYHATSYFPVTLIVEDMKAFDSNRSYVFAVEPHSVLPIGIVALCNHTGYMPLPRIKALASSAIFYTPILRHIWSWLGLVAASRKNFVKYLNSGFSCIVIPGGVREIFYMEYGTEVAFLRRRHGFVRVAIETGCPLVPVFCFGQTEAYRWWRPRGELYNHLARAIRFTPLVFWGKFGSPIPYRRPLQVVVGKPIEVKRNPQPSNEEVVEIHTKFVSALQDLFERHKVPAGYEDTHLRVL

SEQ ID NO: 34 - protein, 322 aa, Vernicia sp.
MGMVEVKNEEEVTIFKSGEIYPTNIFQSVLALAIWLGSFHFILFLVSSSIFLPFSKFLLVIGLLLFFMVIPINDRSKLGQCLFSYISRHVCSYFPITLHVEDINAFRSDRAYVFGYEPHSVFPIGVMILSLGLIPLPNIKFLASSAVFYTPFLRHIWSWCGLTPATRKNFVSLLSSGYSCILVPGGVQETFYMKQDSEIAFLKARRGFIRIAMQTGTPLVPVFCFGQMHTFKWWKPDGELFMKIARAIKFTPTIFWGVLGTPLPFKNPMHVVVGRPIEVKQNPQPTAEEVAEVQREFIASLKNLFERHKARVGYSDLKLEIF

SEQ ID NO: 35 - protein, 327 aa, Elaeis sp.
GDRQPAVFDDSAGAGEAAVYRGTEYSVVRTTVALALWLGLIHFNVALVLTALLLLPARLAAAVFALLLVFMAIPLDENSKWGRKLSRWICKYAVGYFPVTLYVEDIKVFDPNQAYVFAFEPHSVLPIGVVALADLTGFMPLTKVKVLASSAVFYTPFLRHLWTWLGLVSASRKNFYAYLEAGYSCIVVPGGVQEMLHMDHDSEVAFLKARKGFVRIAMETGRPIVPVFCFGQSYVYKWWKPSGKLFVWIARAIKFTPIVFWGRFGSPIPFQHPMQVVVGKPIGLKRNPKPTIEEINEVHAQFVAAFQELFEKHKTRAGYPDFRLRVL

SEQ ID NO: 36 - protein, 333 aa, Zea sp.
MGAGTNNGLSNGAAAGQRADDGTTVFRGTAYSPLRTTVALALWLGAIHFNAFLVLASLFLFPRRVAALVLATQLFFMFLPLSDKSRLGRKIARFISKYVIGYFPVTLHVEDYGAFDPNRAYVFGYEPHSVLPIAVGILGDLVGFMPLPKMKILASSAVFYTPFLRQIWTWLGLAPASRKSFYSYLGAGYSCIIVPGGVQEILHMDHDSEVAFLKPRKGFVKIAIEMGCPVVPVFAFGQSYVYKWWRPGGKLIVKIARAIKFSPIIFWGKLGTPIPFATPMHVIVGRPIEVVKNPQPTIDEINQVHGQFVVAMQDLFEKYKSRTGYPDLQLRVL

SEQ ID NO: 37 - protein, 340 aa, Oryza sp.
MGANGNDVVAAAAAGESPMGAARVVAEGGATVFRGADYSLPRTTVALALWLGGIHFNVFLVLASLFLFPLRVAAMVVAFQLLFMLIPLNDKDKLGRKIARFICRYAMGYFPISLHVEDYKCFDPNRAYVFGFEPHSVLPIGVAALADLVGFMPLPKIKVLASSAVFYTPFLRQIWTWLGLIPATRKNFQSYLGAGYSCIIVPGGVQEILHMDHDSEIAFLKSRKGFVKIAMQSGCPLVPVFCFGQSYAYKWWRPKGKLFVKIARAIKFTPIVFWGRYGTPIPFPTPMHVVVGRPIEVEKNSQPTIDEINEVHEQFTVALQDLFDKYKTETGYPGLHLRVL

SEQ ID NO: 38 - protein, 340 aa, Ricinus sp.
MGEEANHNNNNNNINSNDEKNEEKSNYTVVNSRELYPTNIFHALLALSIWIGSIHFNLFLLFISYLFLSFPTFLLIVGFFVVLMFIPIDEHSKLGRRLCRYVCRHACSHFPVTLHVEDMNAFHSDRAYVFGYEPHSVFPLGVSVLSDHFAVLPLPKMKVLASNAVFRTPVLRHIWTWCGLTSATKKNFTALLASGYSCIVIPGGVQETFYMKHGSEIAFLKARRGFVRVAMEMGKPLVPVFCFGQSNVYKWWKPDGELFMKIARAIKFSPIVFWGVLGSHLPLQRPMHVVVGKPIEVKQNPQPTVEEVSEVQGQFVAALKDLFERHKARVGYADLTLEIL

SEQ ID NO: 39 - protein, 321 aa, Medicago trunculata
MTTATEKVFNGKEEFSDSSNTFRSILALSLWLGSIHFNIAIILFALPFLPLSKYLLVLGFLLICMVIPVDAKSKFGRRLSRYICKHACSYFPITLHVEDIKAFDPNRAYVFSYEPHSVLPIGVIALADNTGFMPIPKVKVLASSAVFYTPFLRHIWTWLGLTPATRKNFISLLAAGYSCILIPGGVQETFLMQRGSEIAYLKARRGFVRIALEKGHPLVPVFCFGQSDIYKWWKPDGKLFLNFSRAIKFTPICFWGIFGSPVPFRHPMHVVVGRPIVLEKNPEPATEEIAKIHSQFVEALQDLFERHKARAGYPKLELKIV

SEQ ID NO: 40 - protein, 317 aa, Brassica sp.
MGKVRDFGAEDHIPSNILHAVTAISICLSAIYLNLALVLFSLFFLPPSLSLLVLGLLSLFIIIPIDDRSKYGLKLARYICKHAASYFPVTLHVEDYEAFKPDRSYVFGYEPHSVWPIGAVALVDLTGFMPLPNIKLLASNAIFYTPFLRHMWAWLGLASASRKSFSSLLESGYSCILVPGGVQETFHLKHDVEDVFLSSRRGFVRIAIEQGAPLVPVFCFGQSRAYKWWKPDCDLYFKLARAIRFTPICFWGVLGSPIPYRHPIHVVVGKPIQVTKSLQPTDEEIDELHGQFVEALKDLFERHKAGAGYSDLQLNIL

SEQ ID NO: 41 - protein, 337 aa, Callithrix sp.
MAFFSRLDLQEGLQTLSVLQWIPVYIFLGAIPILLIPYFLLFSKFWSLAVLSLTWLAYDWNTHSQGGRRSAWIRNWTLWKYFQNYFPIKLVKTHDLSPKHNYIIANHPHGVFSFGVFINFATEATGIARIFPSITPFVGTLEGIFWIPIVREYVMSMGVCPVSRSALKYLLTQKGSGNAVVIVVGGATEALLCRPGTSTLFLKQRKGFVKLALKTGAYLVPSYSFGENDVFNQEKFPEGTWLRLFQKTLQDTVKKILGLNFCTFHGRGFTHGSWGFLPFKRPITTVVGEPLPIPKIERPNQKTVDKYHALYISALRKLFDQHKVEYGLPETQELTIT

SEQ ID NO: 42 - protein, 339 aa, Oryctolagus sp.
MTVTFFSRLDLQEGLQTLSVLQWIPSYIILGAIPILLIPYFLVFTKFWSASVLALAWLVYDWNTHSQGGRRSAWVRNWTLWKYFRNYFPVKLVKTHDLSPKHNYIIANHPHGIVSYGAFINFATEATGFTQMFPSISPSLATLEGIFWIPIVRDYVMSMGLCPVSKLALNYLLTQKGSGNAVIIVVGGATEALLSKPGTSIIFLKERKGFVKLALKTGAYLVPSYTFGENEVHNQTTFPEGTWIRIFQKHFQNIGKRILGINFCTFHGRGLTRGSWGFLPFNRPVTTVVGEPLPVPRINRPSKEVVDKYHALYIRALHKLFDQHKVEYGVPETQELTVI

SEQ ID NO: 43 - protein, 268 aa, Gallus sp.
TGSSILSALQDLFWLSKSKVEKQLQIISVLQWVLTFLIMGIACTLILMYILCTDCWAIAALYLAWLVFDWNTPKKGGRRSQWVRNWAIWRYFRDYFPIRLVKTHNLLTTRNYIFGYHPHGIMGLGAFCNFSTEATGVSQKFPGIRPYLATLAGNFRMPILRDYLMSGGICPVNRDSIDYILSKNGSGNAIIIVVGGAAESLNCTPGKNSVTLRNRKGLGEPITIPKIDNPSQKEVDFYHSVYVDSLIKLFDKYKGRFGLPETEVLEVN

SEQ ID NO: 44 - protein, 245 aa, Ornithorhynchus sp.
LVKTHNLPTNRNYIFGYHPHGIMGLGAFCNFSTEATGVGKKFPGIRPYLATLAGNFRMPVLREYLMSGGICPVNRDTIDYLLSQNGSGNAVIIVVGGAAESLSSVPGRNAVTLRNRKGFVKLALRHGADLVPVYSFGENEVYEQVVFEEGSWGKWVQKKFQKYIGFAPCIFHGRGLFSSNTWGLVPYSKPITTVVGEPITVPKIERPSQHEIDLYHSLYTDALVALFHKYKTRFGLPHTEVLEVN

SEQ ID NO: 45 - protein, 361 aa, Danio sp.
MKTILAAYSGVKKGSGSSILSALHDLPTVPWLTRSKMVKHLQVISVLQFIMTFLTMGIACSLLLMYMFCTDFWVISVLYVAWLIYDWNTPGQGGRRSTWVRDWTVWKYMRDYFPIRLIKTHNLLPSRNYIFGYHPHGILCFGAFCNFGTEATGFTKVFPGIKPSLATLAGNFRLPMFREYLMCGGICPVNRNSIDYLLSSNGTGNAVVIVIGGAAESLDCAPGRNSVMLKKRKGFVKLALKQGADLVPVYSFGENEVYKQLIFEEGSWWRTIQRKLQKFLGFAPCLFHGCGLFFPESWGLVPYCKPITTVVGEPITVPKIEEPTQDVIDMYHAMYIRSLKSLFDNYKTRFGLNESDTLIIH

SEQ ID NO: 46 - protein, 324 aa, Chlamydomonas reinhardtii
MAIDKAPTNVRIWSDGVTEKGKQSIFSSLVAMLTLFIYCGWMHVLLALVILSFWYRWALVTVLLLYSTLLLPPKPVLWGPVCRSWIFQTWREYFKFSYVFDEVLDSKKKYIFAEFPHGVFPMGPLIGATECQIMFPGFDIFGLAANVVFTVPFWRHFVAWLGSVPATTRDFKRVLKQGSVAVIVGGIAEMYMQSPTKEQIMLKDRKGFVRVAVEEGVDGGIVPVYHFGNSQVLDFGPQAMASVSRRLRAALGFLYGVAYLPLPRRRNIYMVCGKPVPVTRTARDDPKFEEVVDATHAAVMAALQEAYDRHKTEYGWADRPLVIS

SEQ ID NO: 47 - protein, 346 aa, Chlamydomonas reinhardtii
MAGGKSNGTGAADAHVRTSHLTLKAGEDPPPNVRIYSDGIKPDARQNLLVQILAGITMSIYVGFMNYFMLLVVLSYWSRICRYVVLALLGTLALPCKPVLWPAFNKLWIFKTWRHYFHYSFLIEEPLDPNKRYIFVEFPHGAFPIGPIVAGTLMQTLFPHMMIYSVAASVVFYIPFWRHFITWIGSVPATPGNFKRLLKKGSVAVVVGGIAEMYMGNKKKERIKLVGRRGFARIALEEQVDGIVCVYYFGQSQVLDFGPSWLADFSRRMRTSFGYLTGWMGLPVPRPIPIYMVNGKPIPVPKVARDSPEFDKEVDKLLDATITELGEMYNRHRGEYGWGDRPLSIE

SEQ ID NO: 48 - protein, 320 aa, Chlamydomonas reinhardtii
MQSKRCAELASGALWPMDRDQMRDRDPWKLRDRAISQAWVWPLLIGTLLYVQSTTLTIAFLLWHIWKVMASYFPGARLIKTADLDPAGRYIFVSHPHGVIAISDWLAFATEALGFSKLFPGLDLRCATLASNFWVPGLREYILSHGMCGVGRDTLARVLTGKPGRAVVLVVGGASEALLAAEGTYDLVLRNRKGFVRLALQTGASLVPVLSYGETDTFHTYIPPPCSRAAAVMKVLKQVFGFSTPLCWGTGLFGGWGMLALQVPLTVVVGAPIQVDKVSSPTEAEVAALHKTYTEALQKLWDDTVDKYGKGVKRPLAIVQ

SEQ ID NO: 49 - protein, 413 aa, Chlamydomonas reinhardtii
MTPRDPPVPRPPPGVRQYTDGRSASYVLPLPYRLLAQLTLGLYVGFPYILLGLLLGTAAGSRAAAAALALTLGSLLVPAPPHIRQGMLDSALFRLWRAYFNYSYAYDQLPDFNRPHIFVNSPHGAFPLSQILCISLSNIVWPGFPVHSLAASVLWYIPLWRHMKAALGAAPASRDNARMLLRHRGSVAVLAGGIAEMYTSSPSRAAAATEPDEAAAAGGAIDTTEAAGATGSSSTTTSPPQPKEQQRDGEQRQGPRKGLKGLLKGPKDDPDPAAEEEQGLGLAPERIKLLGRRGFVRLAVEMGVPIVPIYHMGNSKILTFGPQSLQQLSRRLRMALGAVFGVWGLPVPRPQPLMMCVGSPIPVPYVDPAAEPERFEAVVAAVHGQVVAAFQDLYNRYRVQYGCGWERRPLEVC

SEQ ID NO: 50 - protein, 327 aa, Chlamydomonas reinhardtii
MPLAKLRNVVLEYAAIAIYVSAIYTSVVLLPSALALFYLFGATSPSAWLLLAAFLALTFTPLQLTTGALSERFVQFSVARAAAYFPTRVVVTDPEAFRTDRGYLFGFCPHSALPIALPIAFATTSPLLPKELRGRTHGLASSVCFSAPIVRQLYWWLGVRPATRQSISGLLRARKVAVLVPGGVQEVLNMEHGKEVAYLSSRTGFVRLAVQHGAPLVPVWAFGQTRAYSWFRPGPPLVPTWLVERISRAAGAVPIGMFGQYGTPMPHREPLTIVVGRPIPVPELAPGQLEPEPEVLAALLKRFTDDLQALYDKHKAQFGKGEELVIM

SEQ ID NO: 51 - protein, 526 aa, Homo sapiens
MAVFPSSGLPRCLLTLILLQLPKLDSAPFDVIGPPEPILAVVGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLPRLTPWIVAVAVILMVLGLLTIGSIFFTWRLYNERPRERRNEFSSKERLLEELKWKKATLHAVDVTLDPDTAHPHLFLYEDSKSVRLEDSRQKLPEKTERFDSWPCVLGRETFTSGRHYWEVEVGDRTDWAIGVCRENVMKKGFDPMTPENGFWAVELYGNGYWALTPLRTPLPLAGPPRRVGIFLDYESGDISFYNMNDGSDIYTFSNVTFSGPLRPFFCLWSSGKKPLTICPIADGPERVTVIANAQDLSKEIPLSPMGEDSAPRDADTLHSKLIPTQPSQGAP

SEQ ID NO: 52 - protein, 311 aa, Chlamydomonas reinhardtii
MPNDLLAQVKGKNALYDGEDDNEIEDIEEQVAPPPTDAEREMQEFFKKVELVKTDLAEVKELQKEILSMHEKGKTIVKSKEMQKHRELMQEKIDAVNKLAHACKAKIEALDKDNDAAKKKKGQQAGSASERTRTTITAGLKKKLKDHMQEFSELRTRIQSEYREVVERRVYTVTGTHATDEEIDKMIETGDSENIFQKAILEQGRGRVLDTLAEIQERHRAVKDLEQSLLELHQIFLDMAVLVEAQGEMLDNIEKQVARSVDYVKGGTEALQDAKQLQKNTRKWMCCAIMIMLIVALVIVLAVVRPWKYLQ

SEQ ID NO: 53 - protein, 135 aa, Chlamydomonas reinhardtii
MDAVDRGVYFDEDFHVRILDVDKYNASKSLQDNTNVFINNIQNMQGLVDKYVSAIDQQVERLEAEKLKAIGLRNRVAALSEERKRKQKEQERMLAEKQEELERLQMEEQSLIKVKGEQELMIQKLSDSSSGAAYV

SEQ ID NO: 54 - protein, 437 aa, Homo sapiens
MASVAVDPQPSVVTRVVNLPLVSSTYDLMSSAYLSTKDQYPYLKSVCEMAENGVKTITSVAMTSALPIIQKLEPQIAVANTYACKGLDRIEERLPILNQPSTQIVANAKGAVTGAKDAVTTTVTGAKDSVASTITGVMDKTKGAVTGSVEKTKSVVSGSINTVLGSRMMQLVSSGVENALTKSELLVEQYLPLTEEELEKEAKKVEGFDLVQKPSYYVRLGSLSTKLHSRAYQQALSRVKEAKQKSQQTISQLHSTVHLIEFARKNVYSANQKIQDAQDKLYLSWVEWKRSIGYDDTDESHCAEQFESRTLAIARNLTQQLQTTCHTLLSNIQGVPQNIQDQAKHMGVMAGDIYSVFRNAASFKEVSDSLLTSSKGQLQKMKESLDDVMDYLVNNTPLNWLVGPFYPQLTESQNAQDQGAEMDKSSQETQRSEHKTH

SEQ ID NO: 55 - protein, 268 aa, Chlamydomonas reinhardtii
MAESAGKPLKHLEFVHTYAHKFASGAAYVEGGYQKAKTYVPAVAQPYIAKAEETCLAYAAPLATKATDHAEKILRSTDAQLDALYAASASWLSSSQKLADSNIAAFRGAADKYYDLVKSTAQHVTSKLPTDLSVAKARELLSASLEQAKALADPDAAVAAALDAWTKFAAIPAVAKVLSAASPLTGKGVAAFTAAHDLLVHSALYRYGVSVGASTLGWATSTTPYKLSAAYLYPLVQPVADPALDKVSKSTYVNAAIKYWAPAPVAAA

SEQ ID NO: 56 - protein, 1333 aa, Homo sapiens
MTADKLVFFVNGRKVVEKNADPETTLLAYLRRKLGLSGTKLGCGEGGCGACTVMLSKYDRLQNKIVHFSANACLAPICSLHHVAVTTVEGIGSTKTRLHPVQERIAKSHGSQCGFCTPGIVMSMYTLLRNQPEPTMEEIENAFQGNLCRCTGYRPILQGFRTFARDGGCCGGDGNNPNCCMNQKKDHSVSLSPSLFKPEEFTPLDPTQEPIFPPELLRLKDTPRKQLRFEGERVTWIQASTLKELLDLKAQHPDAKLVVGNTEIGIEMKFKNMLFPMIVCPAWIPELNSVEHGPDGISFGAACPLSIVEKTLVDAVAKLPAQKTEVFRGVLEQLRWFAGKQVKSVASVGGNIITASPISDLNPVFMASGAKLTLVSRGTRRTVQMDHTFFPGYRKTLLSPEEILLSIEIPYSREGEYFSAFKQASRREDDIAKVTSGMRVLFKPGTTEVQELALCYGGMANRTISALKTTQRQLSKLWKEELLQDVCAGLAEELHLPPDAPGGMVDFRCTLTLSFFFKFYLTVLQKLGQENLEDKCGKLDPTFASATLLFQKDPPADVQLFQEVPKGQSEEDMVGRPLPHLAADMQASGEAVYCDDIPRYENELSLRLVTSTRAHAKIKSIDTSEAKKVPGFVCFISADDVPGSNITGICNDETVFAKDKVTCVGHIIGAVVADTPEHTQRAAQGVKITYEELPAIITIEDAIKNNSFYGPELKIEKGDLKKGFSEADNVVSGEIYIGGQEHFYLETHCTIAVPKGEAGEMELFVSTQNTMKTQSFVAKMLGVPANRIVVRVKRMGGGFGGKETRSTVVSTAVALAAYKTGRPVRCMLDRDEDMLITGGRHPFLARYKVGFMKTGTVVALEVDHFSNVGNTQDLSQSIMERALFHMDNCYKIPNIRGTGRLCKTNLPSNTAFRGFGGPQGMLIAECWMSEVAVTCGMPAEEVRRKNLYKEGDLTHFNQKLEGFTLPRCWEECLASSQYHARKSEVDKFNKENCWKKRGLCIIPTKFGISFTVPFLNQAGALLHVYTDGSVLLTHGGTEMGQGLHTKMVQVASRALKIPTSKIYISETSTNTVPNTSPTAASVSADLNGQAVYAACQTILKRLEPYKKKNPSGSWEDWVTAAYMDTVSLSATGFYRTPNLGYSFETNSGNPFHYFSYGVACSEVEIDCLTGDHKNLRTDIVMDVGSSLNPAIDIGQVEGAFVQGLGLFTLEELHYSPEGSLHTRGPSTYKIPAFGSIPIEFRVSLLRDCPNKKAIYASKAVGEPPLFLAASIFFAIKDAIRAARAQHTGNNVKELFRLDSPATPEKIRNACVDKFTTLCVTGVPENCKPWSVRV

SEQ ID NO: 57 - protein, 244 aa, Homo sapiens
MELTIFILRLAIYILTFPLYLLNFLGLWSWICKKWFPYFLVRFTVIYNEQMASKKRELFSNLQEFAGPSGKLSLLEVGCGTGANFKFYPPGCRVTCIDPNPNFEKFLIKSIAENRHLQFERFVVAAGENMHQVADGSVDVVVCTLVLCSVKNQERILREVCRVLRPGGAFYFMEHVAAECSTWNYFWQQVLDPAWHLLFDGCNLTRESWKALERASFSKLKLQHIQAPLSWELVRPHIYGYAVK

SEQ ID NO: 58 - protein, 213 aa, Arabidopsis thaliana
MGCFHSKAAKEFRGHEDPVKLASETAFSVSEVEALFELFKSISSSVVDDGLINKEEFQLALFKSRKRENIFANRIFDMFDVKRKGVIDFGDFVRSLNVFHPNASLEDKIDFTFRLYDMDCTGYIERQEVKQMLIALLCESEMKLADETIEIILDKTFEDADVNQDGKIDKLEWSDFVNKNPSLLKIMTLPYLRDITTTFPSFVFHSEVDEIAT

SEQ ID NO: 59 - protein, 1142 aa, Chlamydomonas reinhardtii
MDSKEAAEPASVHVNVDVEAQKAQAQAEAAAKGGACATSGMSKGKIIVTSLVIFLGVAVGVGLGVGLGVGLKKDDGSSAYTSLDLGTGSGGGNTYFVAADKIQWNYAPSGRNKCFPPDLAAKYLAMQPGITRVGGTFAKAIYRAYTDSSFNTLATTPAEWQHLGNVGPVMYGAVGQVIRVVFKNNLDFPVNMAPSGGLIAWDGNGRRSARIDPVKPGQTVTYLWQIPEDAGPVANATVTSRLWLYRSSVDPQKHDNAGLVGPIIVTSAANADANGRARDVDRDVVAIFQIVQERASPLLFQEDTSLSAGTSYTKMAINGYTWCNMPDGAITIKTGERVRWHVASIGSSESLHNFHWHGHVVELNGHHVDQFTAIPTATYSVNMVPDEPGTWMFHCHVNFHMDGGMVALYTVTGDPAPLPTGGVERVYYVRAQEVEWSYSGPNNTQACAVPELQFSSEPGSEEVNGNVFLEGPSTDPVRLGHIYTKTLLIEYTDASFTTVKPRPADEQYLGLLGPVMRANVGDTIKVVLKNDAKIDVSLHPHGVRYSKANEGTLYEDGTSGADKADDVVAPGTTYTYVWNVPDRAGPGPCDPSSMLWMYHSHIDETAETYAGVAGGIIVTAKDMARSTADLTPKDVDREIVIFFTVVDEIKSSNFMENLANKLGDGGALAAQLAANATEMTALVTDPVFMEHMLKHGINGHMYCHMPRLTFEQGDKVRLHVMVLGTLEDMHTPNMGGPRFDYNGMHTDSIQISPGGMVSADVQMTSPGDYELQCRVADHVMAGMRAKYTVTANASRMVVNPSGVTRTYYIQAEAVNWDYAPAGYQKCTDTDFSYQSSVYLRRTSYTIGSRYRKAVYRAYTDATFSTRVPTPAYYGTMGPMIIAEVGDRIVVHFKNAVTDLEEYPLNISPGGGLLVEGAADENCAEVAAGETCVYRWIVPDSSGPGTADFNTAVYGYTSSVDVATAPSAGLAGALVVAGRGQLVAGPDGSLLPRGVDLMVPLYWQVVDENSSPFLDLNVEAAQLNVTKFENDAVLSADFDEGNRMHSINGYVYCNQPLVTIAKGKKLRWVLVAYGTEGDFHSPQFTGQSLEADKSGYSTLASLMPSIARVADMTAADVGTWLLYCDVHDHYMAGMMSQFAVTAA

SEQ ID NO: 60 - protein, 4176 aa, Chlamydomonas reinhardtii
MPDGYATNMTICGAAVQLLSAAGAVKASPFSACLLAADTLTLTLTSSGTYAPGDVVNVVAGQSTLKNGATSYTKSAAGSVIRPTLTTVSLATTTTILIGASAPVSLPANASADVCNSIFTVALKSGTALPTALSACMAGPAVTAITATFANGTTYSDGLTVTIKANQTLLRTGDQSASSPLFLPPASALVIAPTVLSASLTSATSITVQLPVSSTASGTLDAAGCNGAFELRAAGSSASKSSPFSACALASNNTALVLTLASASTYTAGDIFNVKSGQSLLQVGSTAYVPQAVSVLPTLSTAILVAASVVRVGLPVVSSIPAPYSADDCARSVIIAAANSTAAKAISGCVLSADGKALLVTLGAAYAAGDTVNVRTGQWQLRAGASVTLGPNYIAASTPVAIVPGFSSAVLTSATQVTVQLPLASALPATISASECGDAFDLLDASGTTSRVSPWTGCSIGSNNTLVLNMTAGIYVVGDQVTPKSGQTKLTFANTTAYGVLLTPINPILTAATTAMLTASSTIVVALPYAANLPASTNNGTVCNAAFDLVSAAGASRTAPFSACSISGGTTLTLTIASTASYTAGDRINVKSGNSAFLGSLSASGPAFVHAGTAIVITPTVVSTALISNTNVFVSLSAPTSASAFNQSICNGAFDVSGLATPFTNCTLSADGTGISFLLASAGSVTSGVTTINVKSSQLFLLAAGSGSSDSPAFVPRGSALTISEAKLAGAYLTAANTIIVKLSVAAAAVDGFSSSCNNVFTLFNSSGTARASPFSACTLSTDGLTVTLTTSSWVSTDKLNIRSDQTLLKVLGAANGPSYPALSSATEVSPAITSAHITSPTTIIVPLPVASDLTGSSCSFLVLTAATSNCALSNNGTLLTVTLSGSYTIGDTININALNSALRGTSSTGALYQPVSGATAATIQPTIGAVQVTTSTRLLVTLPAAMSSPVPNPLTADACNAALDLSGKSSPFAACTASGTTLTLDLASAFVPGDRLNVKSTNTALRLGTGASALFFQPLATSPANILNPTFVSAKATSTSVVVVSLPAASTFVKSGSATAGLVKADCDTVLAMSSGSLVGSGNACDLNTTSSSQLIVTLAGTTYAPGQTINVLTTNTMLLAGSSSGPAYQPRTITINPAYLSSDVVATAPDTVVVTLPVTSGLFAADGSSLGSTLTAAQCATVLEVKAGTTAKGLASCSLAGTTLTVKLLGNNSDVYTGGDTFNFKDTNALLLAGSANTAPAYKALATAAVIVPNLYKAVASAGDTILITLPAASTFVVSGSAVLSVSDTVCNTILTFTNSKTVKSGTNCVITGAVLSLTLNSAITDQTTVTINSGGQTTLVSGTGTTGPAYKAGTAAPISPAYLTSAAARSATSIVVTLPFTSTVNGATLTKATCDTIVEVLNGGDATKPRTLDSGTPCTLATTLLTVNLAATESFAPGDTVRIKSGNTVLLIGSAGGNAPYVQATTATPIALGYVTSSIATKATEIKVTLPVPITLIKAGVSVASLDKTDCDTLLTVASGTLAATGSCALSGTVLTITTTSTYTPGTTTVQFTAVASSASVKILGGAGTTGTIIAALSAASSVLPGYLTSAVISGANTFTLTLPYAVTSGGNPTCSDIIEIKAANGAMKTFNGVCSVASATTVTGTTNEALLASDTVTLKGAQNALTTTTGSKIYAATAAINIQPAYLTGAYAIDDKKFVVVLPYASTLAAGTCSSIVSVTDNNNGPAESGCSLALDGTGIYLTVTVSASLTATAWKVDFKAAQTALTVGSTAWAPMATGTAKSLNAGATTWGSSIMTATAVATNVLEVTLPMSSYMADISSGCSPVTFSTANTAASCKLIGTTSSPNSILQITLTAGYVAGAVSLANAAGLIKAGSASGTAIGAASTVTIKPTFVSAVATGPRTIVVALPIQSKIIKGAPTCTGGAGTTGDTCDDDIASPADCSTIFSLTGGTATIAGCSNPGSDFTAPYTTTVTLATANWVPGTTLSVAADQAAAAATNYLKPTAAITLLYTTKPSGAVVIYPSISSATLTGPKSLQVTLPVAASVPSSGLSPADCNNIFQLYATGTTTPKTAAVFSACYLGADKQTFYLTLAAAAASTTNANTYAVKDLINIKAGQSLLKADSATAANRLAFAPLPDALLIRPAITSAVATTIASAAVIKMQLPLTSNLGTVNCATTGIKVRVDDATDKTQANTGACSISSDANGAVLTLTLNQALGASDFTTGKGVSVVVVATSSADATKLQLFGGSDNTGPLYVTGSIPVYDVVTPGQSIVSAKAIASNQVEIKLPAPSTITTGNTCANFLAFTPTRTISSCVYDAETQLVTATVDQFAAGDAVNIAGAPNLLKYATAAATLTDYAALAAAATVSPALVSAYTVDSTTIAVVLPATSSFYSGATNSATKVASLTDVECADVLTVLLAGKTNSAGVSACATPSNCRTLFSGAACLLGTTSAGDTLLTIKLGASYAAGDAVDVWDKNAGLNGATAAAKPLRVGTNVQALYVPRRMPVVIEPRIVSASATGARTIAVTLPVTSQLIDSTGTVISAPTQQQCASLVALPAGKSVASCAISSDFLTLTLTLAADFAPGDTVNIASGQTLLRAWKSDVGPGYELSGPLYTPSASAVTVTLSFFVSATSTAANTIVVTLPFPVKMYDLAASPAEILAADGATPTKDNCDSVIRVIPSGSTIARTLVAPGAGVAPCVLSGSGTTITLTLDTTVSSYATGDRIDVGFGNSANLRGAATAAAVANTSPKYTTVATLNAAASAAVVTIGPSIVSAAAASPTQVKVTLSATGTITTTPATVDDCNKIVTFTPAKTLAATSPCSVAGTTLTVNLDKATPYAGTDAVNIAAANSLAATTNPLKAGSLAFVAKATAVTIDPNLYTATAIDSLVYEVALPAASSVGSTALTAAQCGAILSLTGGRTISSTVTAPCVLDTTKTLLTVSLGTAYADGDTVTLQSTQATPWLRAASDTGPAYKVGSTLIVKPTIASAKATTATTIEVTLPVTSVIWSTSGGAAVSTALTATECGKILSVKRGSGTVALSASGACSLSSSGSTTLTVTLASDQVFQAGDKINILASNTDSANTDKYLVATTSSSGQAYIARSSDVVIAPGFINAAYAVGPNTLSVALPVVSSIATDNDCSTVVTVRASATPTTTRTVSGACTVASDATGYYLVVTLAAGTPFVSGDEVLIKSGNTALVGSIAYASGAAGATKPIYANFDDAILTAPTTVLVTMAAVTTVSFTSKADCDAVFVFSGAGNTTKTTSPIASCALAPDGLSVTITLSSADAYVPGDAINVVKDQTSAKVGGVGGTNLVPYPTATTISPRPFGAKATLVAPTSVAFSLPAVSSLPAATAAADCNAAVRYTDVAGVDAVNPFTSCALTPDTKGVVLNTASPIYKAGDVMNIKSGNAAVRWGPQASGAAYYPAAADVPVFATVATATLVDPSTVVLNLPTVSAIPDNFTVPDCLRAIEFKTAAGVTKNGTVASCMLMPDRLGLQVKLLSAGNFSAGDTVNIKPEQAELRSSALATGPSYVPKLAAQVVNPALFANANLTSATAITVRLPFASALATGADCKAVLALLGAGGVAKNVSSCSLGADGVTLAVTIPAASFVGGDVLNIVPGQRALTLADGTTAYVPSKAGVLVTPNIVSATLTNATIVTVALPTASVLTSTAAADCNAAVVIARNGSAVASPLSACAVSADGLSLTLTAAATYKPMAGDTVDVAVSQTVLRAGSATGPAYVPRPSPALITVPSPPPSPPPSPAPSPPPSPPLSTANYSARGLATGPLSCNVLIGDLTVTTTAGNFTGMASYAGKDASYKGSCKDAVTGAVYTDDTVASTLPAGLTGLVLSPVTALASVWDLKSVADLANTNKDYLRLFGVPVNASAYGTAVQLLAYDYYVKGFVALETPAVAVLNVEGMVAANLLMYTKFFDGLNTSVAGRDITAAQALAAGQYALAAVLEDSIAPINTTDPASILALLNVTYSVLTANATSAAGRRLLQTAPAPFTQLQAQATALAAAAAGSNALVAAQQARLITAINAGTSISAAELTNIINEASKVIVAQSTVIATAATGLGSGAISPASFTSSYTGSALSTLVAQQQLAATPGSDVTAGPAPSPPSDKKKSNTGLIVGLVVGLVGGAIVITLIVVFIVMRRRKQNVAAAGQATA

SEQ ID NO: 61 - protein, 6 aa, Artificial Sequence
  Description of Artificial Sequence Synthetic 6xHis tag
HHHHHH